(19) United States
(12) Patent Application Publication
(10) Pub. No.: US 2023/0127326 A1
(43) Pub. Date: Apr. 27, 2023

(54) C3-SUBSTITUTED 1H-PYRAZOLO[4,3-d]PYRIMIDINE COMPOUNDS AS TOLL-LIKE RECEPTOR 7 (TLR7) AGONISTS

(71) Applicant: BRISTOL-MYERS SQUIBB COMPANY, Princeton, NJ (US)

(72) Inventors: Christine M. TARBY, Lawrenceville, NJ (US); Qiang CONG, Palo Alto, CA (US); Ashvinikumar V. GAVAI, Princeton Junction, NJ (US); Sanjeev GANGWAR, Foster City, CA (US); Matthias BROEKEMA, New Hope, PA (US); Patrice GILL, Levittown, PA (US); Prasanna SIVAPRAKASAM, Plainsboro, NJ (US); Walter L. JOHNSON, San Francisco, CA (US); Murugaiah Andappan Murugaiah SUBBAIAH, Bangalore (IN); Yam B. POUDEL, Fremont, CA (US)

(21) Appl. No.: 17/792,905

(22) PCT Filed: Jan. 26, 2021
(86) PCT No.: PCT/US2021/014981

§ 371 Date: Jul. 14, 2022

Related U.S. Application Data
(60) Provisional application No. 63/057,686, filed on Jul. 28, 2020.
(60) Provisional application No. 62/966,103, filed on Jan. 27, 2020.

(51) Int. Cl. A61K 31/519; A61K 39/395; C07D 487/04

(52) U.S. Cl. CPC A61K 31/519; A61K 39/3955; C07D 487/04

(57) ABSTRACT

Compounds according to formula I are useful as agonists of Toll-like receptor 7 (TLR7). Such compounds can be used in cancer treatment, especially in combination with an anti-cancer immunotherapy agent, or as a vaccine adjuvant.

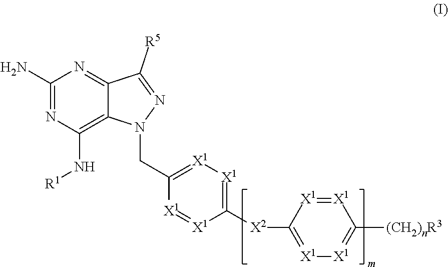

### CROSS-REFERENCE TO RELATED APPLICATIONS

This application claims the benefit under 35 U.S.C. § 119(e) of U.S. Provisional Application Ser. No. 63/057,686, filed Jul. 28, 2020, and U.S. Provisional Application Ser. No. 62/966,103, filed Jan. 27, 2020; the disclosures of which are incorporated herein by reference.

### BACKGROUND OF THE DISCLOSURE

This disclosure relates to Toll-like receptor 7 (“TLR7”) agonists and conjugates thereof, and methods for the preparation and use of such agonists and their conjugates.

Toll-like receptors (“TLRs”) are receptors that recognize pathogen-associated molecular patterns (“PAMPs”), which are small molecular motifs conserved in certain classes of pathogens. TLRs can be located either on a cell's surface or intracellularly. Activation of a TLR by the binding of its cognate PAMP signals the presence of the associated pathogen inside the host—i.e., an infection—and stimulates the host's immune system to fight the infection. Humans have 10 TLRs, named TLR1, TLR2, TLR3, and so on.

The activation of a TLR—with TLR7 being the most studied—by an agonist can have a positive effect on the action of vaccines and immunotherapy agents in treating a variety of conditions other than actual pathogen infection, by stimulating the immune response overall. Thus, there is considerable interest in the use of TLR7 agonists as vaccine adjuvants or as enhancers in cancer immunotherapy. See, for example, Vasilakos and Tomai 2013, Sato-Kaneko et al. 2017, Smits et al. 2008, and Ota et al. 2019.

TLR7, an intracellular receptor located on the membrane of endosomes, recognizes PAMPs associated with single-stranded RNA viruses. Its activation induces secretion of Type I interferons such as IFNα and IFNβ (Lund et al. 2004). TLR7 has two binding sites, one for single stranded RNA ligands (Berghöfer et al. 2007) and one for small molecules such as guanosine (Zhang et al. 2016).

TLR7 can bind to, and be activated by, guanosine-like synthetic agonists such as imiquimod, resiquimod, and gardiquimod, which are based on a 1H-imidazo[4,5-c]quinoline scaffold. For a review of small-molecule TLR7 agonists, see Cortez and Va 2018.

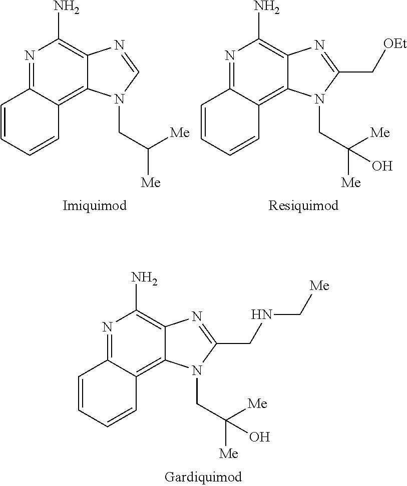

Synthetic TLR7 agonists based on a pteridinone molecular scaffold are also known, as exemplified by vesatolimod (Desai et al. 2015).

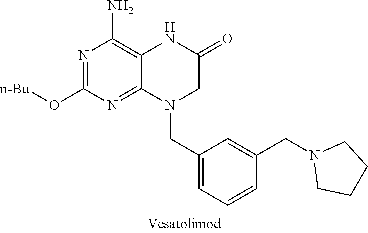

Other synthetic TLR7 agonists based on a purine-like scaffold have been disclosed, frequently according to the general formula (A):

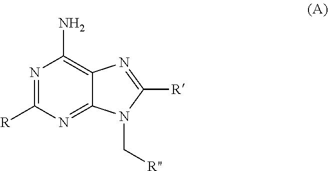

where R, R′, and R″ are structural variables, with R″ typically containing an unsubstituted or substituted aromatic or heteroaromatic ring.

Disclosures of bioactive molecules having a purine-like scaffold and their uses in treating conditions such as fibrosis, inflammatory disorders, cancer, or pathogenic infections include: Akinbobuyi et al. 2015 and 2016; Barberis et al. 2012; Carson et al. 2014; Ding et al. 2016, 2017a, and 2017b; Graupe et al. 2015; Hashimoto et al. 2009; He et al. 2019a and 2019b; Holldack et al. 2012; Isobe et al. 2009a and 2012; Poudel et al. 2019a and 2019b; Pryde 2010; and Young et al. 2019.

The group R″ can be pyridyl: Bonfanti et al. 2015a and 2015b; Halcomb et al. 2015; Hirota et al. 2000; Isobe et al. 2002, 2004, 2006, 2009a, 2009b, 2011, and 2012; Kasibhatla et al. 2007; Koga-Yamakawa et al. 2013; Musmuca et al. 2009; Nakamura 2012; Ogita et al. 2007; and Yu et al. 2013.

There are disclosures of related molecules in which the 6,5-fused ring system of formula (A)—a pyrimidine six member ring fused to an imidazole five member ring—is modified. (a) Dellaria et al. 2007, Jones et al. 2010 and 2012, and Pilatte et al. 2017 disclose compounds in which the pyrimidine ring is replaced by a pyridine ring. (b) Chen et al. 2011, Coe et al. 2017, Poudel et al. 2020a and 2020b, and Zhang et al. 2018 disclose compounds in which the imidazole ring is replaced by a pyrazole ring. (c) Cortez et al. 2017 and 2018; Li et al. 2018; and McGowan et al. 2016a, 2016b, and 2017 disclose compounds in which the imidazole ring is replaced by a pyrrole ring.

Bonfanti et al. 2015b and 2016 and Purandare et al. 2019 disclose TLR7 modulators in which the two rings of a purine moiety are spanned by a macrocycle:

A TLR7 agonist can be conjugated to a partner molecule, which can be, for example, a phospholipid, a poly(ethylene glycol) (“PEG”), an antibody, or another TLR (commonly TLR2). Exemplary disclosures include: Carson et al. 2013, 2015, and 2016, Chan et al. 2009 and 2011, Cortez et al. 2017, Gadd et al. 2015, Lioux et al. 2016, Maj et al. 2015, Vernejoul et al. 2014, and Zurawski et al. 2012. A frequent conjugation site is at the R″ group of formula (A).

Jensen et al. 2015 discloses the use of cationic lipid vehicles for the delivery of TLR7 agonists.

Some TLR7 agonists, including resiquimod are dual TLR7/TLR8 agonists. See, for example, Beesu et al. 2017, Embrechts et al. 2018, Lioux et al. 2016, and Vernejoul et al. 2014.

Full citations for the documents cited herein by first author or inventor and year are listed at the end of this specification.

### BRIEF SUMMARY OF THE DISCLOSURE

This specification relates to compounds having a 1H-pyrazolo[4,3d]pyrimidine aromatic system wherein the C3 carbon (arrow) of the pyrazole ring is substituted (i.e., is other than H), having activity as TLR7 agonists.

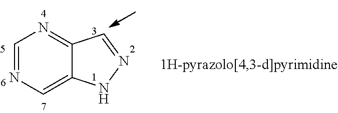

In one aspect, there is provided a compound with a structure according to formula I

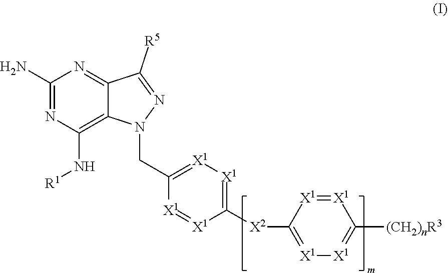

wherein
each X¹ is independently N or CR²;
X² is O, CH₂, NH, S, or N(C₁-C₃ alkyl);
R¹ is (C₁-C₅ alkyl),
   (C₂-C₅ alkenyl),
   (C₁-C₈ alkanediyl)₀₋₁(C₃-C₆ cycloalkyl),
   (C₂-C₈ alkanediyl)OH,
   (C₂-C₈ alkanediyl)O(C₁-C₃ alkyl),
   (C₁-C₄ alkanediyl)₀₋₁(5-6 membered heteroaryl),
   (C₁-C₄ alkanediyl)₀₋₁phenyl,
   (C₁-C₄ alkanediyl)CF₃,
   (C₂-C₈ alkanediyl)N[C(═O)](C₁-C₃ alkyl),
   or
   (C₂-C₈ alkanediyl)NRR^{y};
each R² is independently H, O(C₁-C₃ alkyl), S(C₁-C₃ alkyl), SO₂(C₁-C₃ alkyl), C₁-C₃ alkyl, O(C₃-C₄ cycloalkyl), S(C₃-C₄ cycloalkyl), SO₂(C₃-C₄ cycloalkyl), C₃-C₄ cycloalkyl, C₁, F, CN, or [C(═O)]₀₋₁NR^{x}R^{y};
R³ is NH(C₁-C₅ alkyl), N(C₁-C₅ alkyl)₂, NH(C₁-C₄ alkanediyl)₀₋₁(C₃-C₆ cycloalkyl), N(C₃-C₆ cycloalkyl)₂, NH(C₁-C₄ alkanediyl)₀₋₁(aryl), or a cyclic amine moiety having the structure

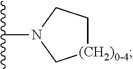

R⁵ is H, C₁-C₅ alkyl, C₂-C₅ alkenyl, C₃-C₆ cycloalkyl, halo, O(C₁-C₅ alkyl), (C₁-C₄ alkanediyl)OH, (C₁-C₄ alkanediyl)O(C₁-C₃ alkyl), phenyl, NH(C₁-C₅ alkyl), 5 or 6 membered heteroaryl,

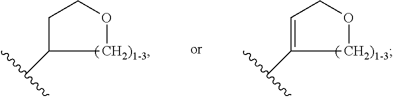

R^{x} and R^{y} are independently H or C₁-C₃ alkyl or R^{x} and R^{y} combine with the nitrogen to which they are bonded to form a 3- to 7-membered heterocycle;
m is 0 or 1;
and
n is 1, 2, or 3;
   wherein in R¹, R², R³, and R⁵
   an alkyl moiety, alkanediyl moiety, cycloalkyl moiety, or a moiety of the formula

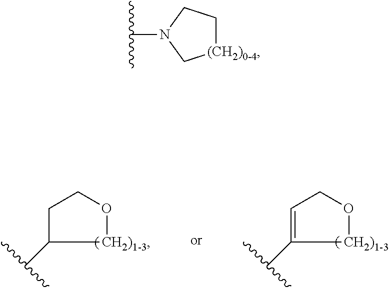

is optionally substituted with one or more substituents selected from OH, halo, CN, (C₁-C₃ alkyl), O(C₁-C₃ alkyl), C(═O)(C₁-C₃ alkyl), SO₂(C₁-C₃ alkyl), NR^{x}R^{y}, (C₁-C₄ alkanediyl)OH, (C₁-C₄ alkanediyl)O(C₁-C₃ alkyl);
and
an alkyl, alkanediyl, cycloalkyl, or a moiety of the formula

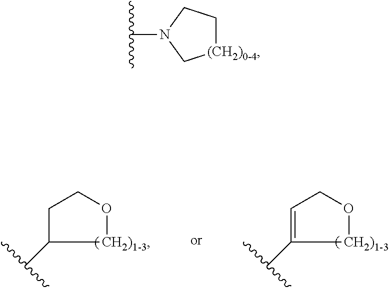

may have a CH₂ group replaced by O, SO₂, CF₂, C(═O), NH,
N[C(═O)]₀₋₁(C₁-C₃ alkyl),
N[C(═O)]₀₋₁(C₁-C₄ alkanediyl)CF₃,
N[C(═O)]₀₋₁(C₁-C₄ alkanediyl)OH,
or
N[C(═O)]₀₋₁(C₁-C₄ alkanediyl)₀₋₁(C₃-C₅ cycloalkyl).

Compounds disclosed herein have activity as TLR7 agonists and some can be conjugated to an antibody for targeted delivery to a target tissue or organ of intended action. They can also be PEGylated, to modulate their pharmaceutical properties.

Compounds disclosed herein, or their conjugates or their PEGylated derivatives, can be used in the treatment of a subject suffering from a condition amenable to treatment by activation of the immune system, by administering to such subject a therapeutically effective amount of such a compound or a conjugate thereof or a PEGylated derivative thereof, especially in combination with a vaccine or a cancer immunotherapy agent.

### DETAILED DESCRIPTION OF THE DISCLOSURE

### Compounds

In one aspect, compounds of this disclosure are according to formula (Ia), wherein R¹, R³, and R⁵ are as defined in respect of formula (I):

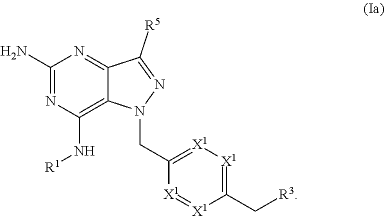

In another aspect, compounds of this disclosure are according to formula (Ib), wherein R¹, R³, and R⁵ are as defined in respect of formula (I):

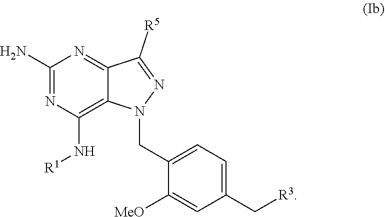

In one aspect, this disclosure provides a compound having a structure according to formula (Ib) wherein

### R¹ is

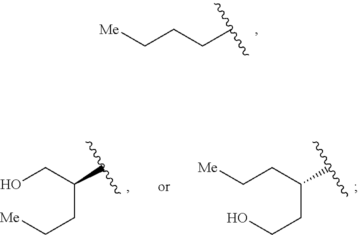 [SMILES: *CCCC ; *[C@H](CO)CCC ; *[C@@H](CCC)CCO]

### R³ is

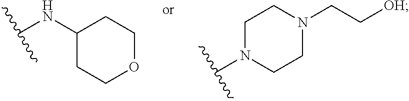 [SMILES: *NC1CCOCC1 ; *N1CCN(CCO)CC1]

and
R⁵ is Me or CH₂OH.

In another aspect, there is provided a compound according to formula (Ic)

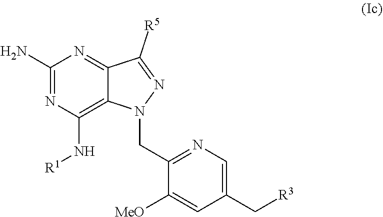

wherein R¹, R³ and R⁵ are as defined in respect of formula (I).

Examples of groups R¹ are

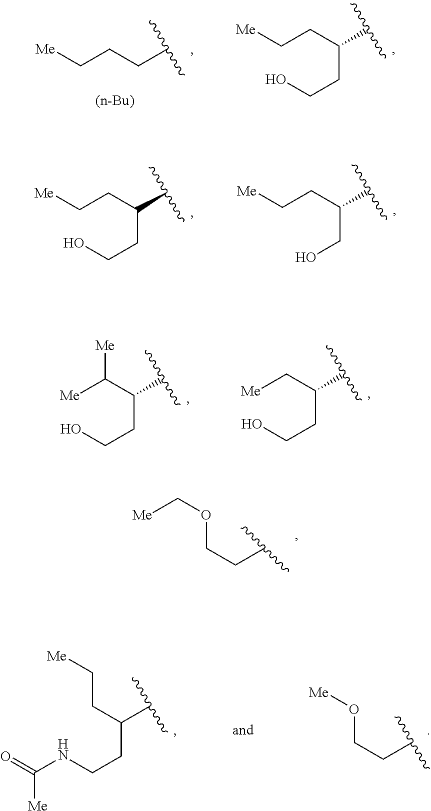

Preferably, R¹ is

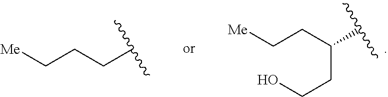 [SMILES: *CCCC ; *[C@@H](CCC)CCO]

Examples of groups R³ include Cl, OH,

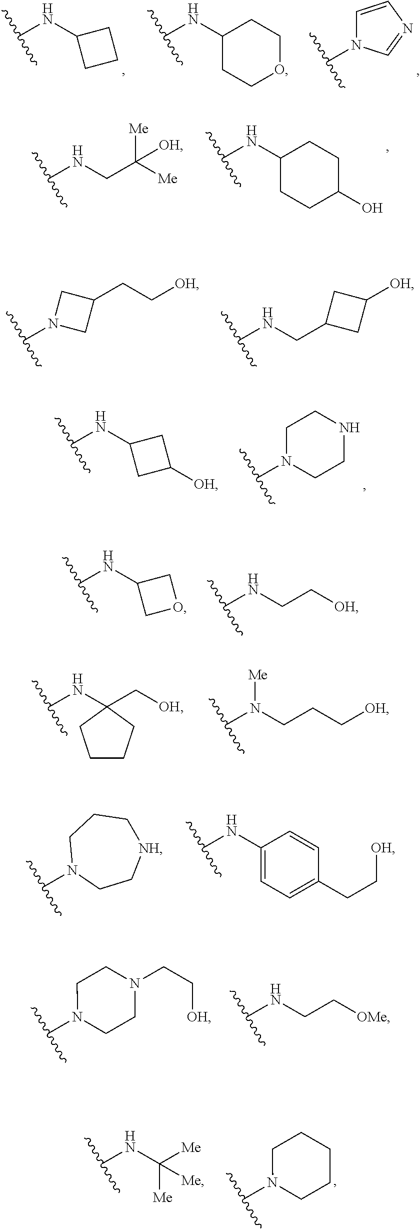

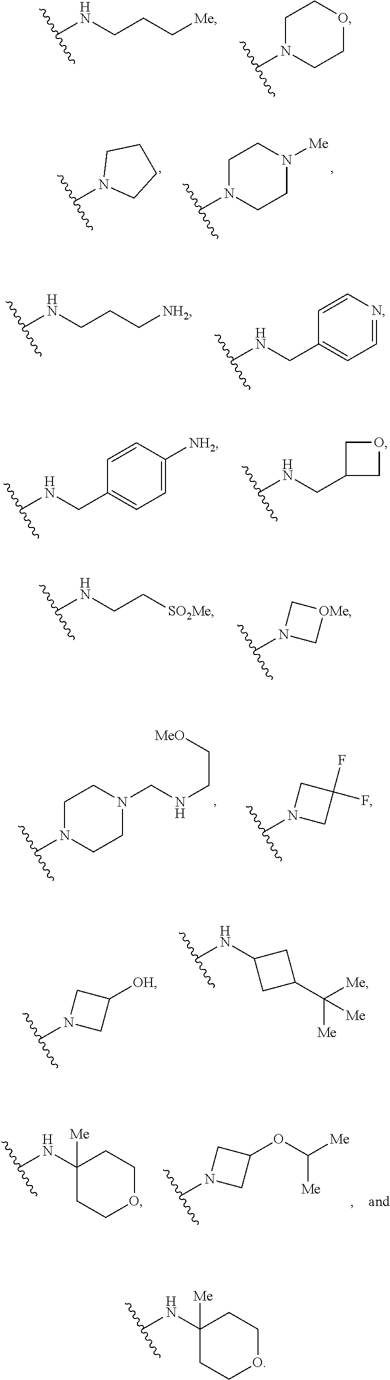

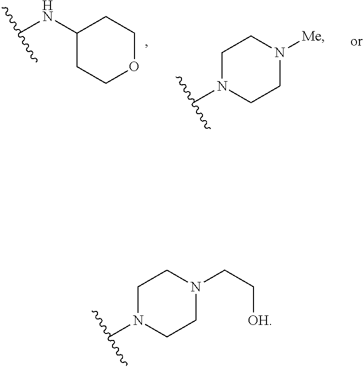 [SMILES: *NC1CCOCC1 ; *N1CCN(C)CC1 ; *N1CCN(CCO)CC1]

Examples of groups, R⁵ are

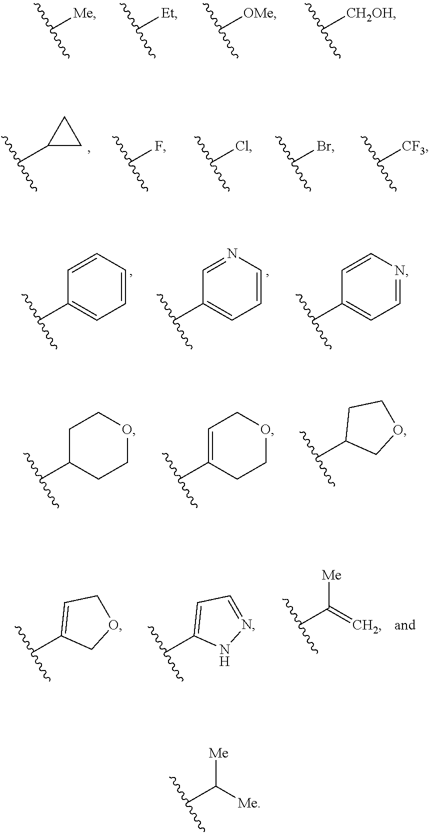

Preferably, R⁵ is

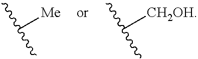

By way of exemplification and not of limitation, moieties of the formula

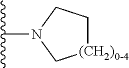 [SMILES: *N1CCCC1]

include

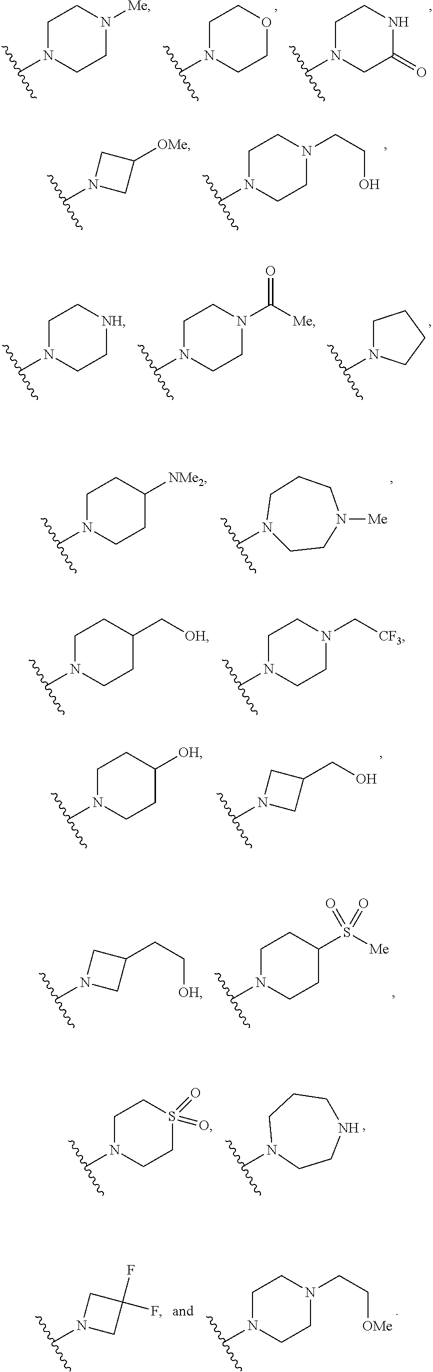

Some of the above exemplary moieties of the formula

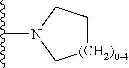 [SMILES: *N1CCCC1]

bear optional substituents and/or optionally have one or more CH₂ groups replaced by O, SO₂, etc., as described in the BRIEF SUMMARY OF THE DISCLOSURE above.

Specific examples of compounds disclosed herein are shown in the following Table A. The table also provides data relating to biological activity: human TLR7 (hTRL7) agonism reporter assay and/or induction of the CD69 gene in human whole blood, determined per the procedures provided hereinbelow. The right-most column contains analytical data (mass spectrum, LC/MS retention time, and NMR). In one embodiment, a compound of this disclosure has (a) a human TLR7 (hTLR7) Reporter Assay EC₅₀ value of less than 1,000 nM and (b) a human whole blood (hWB) CD69 induction EC₅₀ value of less than 1,000 nM. (Where an assay was performed multiple times, the reported value is an average.)

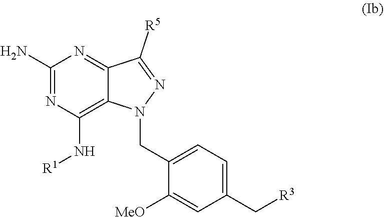

TABLE A
Compounds According to Formula (Ib)

| | | hTLR7 | hWB | Analytical Data |
|---|---|---|---|---|
| Cpd. | | Agonism | CD69 | (Mass spec, LCMS Retention Time, |
| No. | Structure | EC₅₀ (nM) | EC₅₀ (nM) | ¹H NMR (500 MHz, DMSO-d₆)) |
| 101 | 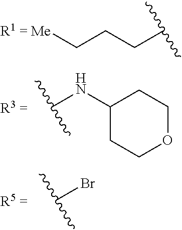 | 2000 | 365.4 | LC/MS (M + H)⁺ 518.3 LC/MS RT (min): 1.45 (UPLC Method B) δ 7.07 (s, 1H), 6.86 (br d, J = 8.0 Hz, 1H), 6.74 (br t, J = 4.6 Hz, 1H), 6.63 (d, J = 7.7 Hz, 1H), 5.97 (s, 2H), 5.59 (s, 2H), 3.88-3.73 (m, 6H), 3.24 (br t, J = 11.4 Hz, 1H), 2.75-2.60 (m, 1H), 1.84-1.73 (m, 2H), 1.52 (quin, J = 7.2 Hz, 2H), 1.40-1.15 (m, 4H), 0.87 (t, J = 7.4 Hz, 3H) |
| | 3-bromo-N7-butyl-1-[(2-methoxy-4- | | | |
| | {[(oxan-4-yl)amino]methyl}phenyl)- | | | |
| | methyl]-1H-pyrazolo[4,3- | | | |
| | d]pyrimidine-5,7-diamine | | | |
| 102 | 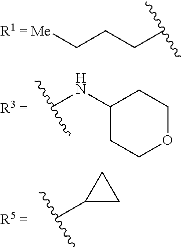 | 210.5 | 34.5 | LC/MS (M + H)⁺ 480.2 LC/MS RT (min): 1.46 (UPLC Method B) δ 7.12 (br s, 1H), 6.84 (br d, J = 8.2 Hz, 1H), 6.48-6.41 (m, 1H), 6.38 (d, J = 7.7 Hz, 1H), 5.65 (br s, 2H), 5.51 (s, 2H), 3.85 (s, 6H), 3.31-3.20 (m, 1H), 2.09-1.98 (m, 1H), 1.85 (br d, J = 12.2 Hz, 2H), 1.56-1.44 (m, 2H), 1.43-1.32 (m, 2H), 1.28-1.11 (m, 3H), 1.02-0.93 (m, 2H), 0.91-0.80 (m, 5H) |
| | N7-butyl-3-cyclopropyl-1-[(2- | | | |
| | methoxy-4-{[(oxan-4-yl)amino] | | | |
| | methyl}phenyl)-methyl]-1H- | | | |
| | pyrazolo[4,3-d]pyrimidine-5,7- | | | |
| | diamine | | | |
| 103 | 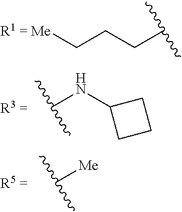 | 101.3 | 15.4 | LC/MS [M + H]⁺ = 424.2 RT (min) = 1.23 (LC/MS Method 1) LC/MS [M + H]⁺ = 424.0 RT (min) = 0.98 (LC/MS Method 2) δ 7.01 (s, 1H), 6.75 (d, J = 7.6 Hz, 1H), 6.44 (d, J = 7.8 Hz, 1H), 6.40-6.33 (m, 1H), 5.60 (s, 2H), 5.51 (s, 2H), 3.83 (s, 3H), 2.23 (s, 3H), 2.08-1.97 (m, 2H), 1.75-1.38 (m, 6H), 1.30-1.12 (m, 2H), 0.85 (t, J = 7.4 Hz, 3H) |
| | N7-butyl-1-({4-[(cyclobutylamino) | | | |
| | methyl]-2-methoxyphenyl}methyl)-3- | | | |
| | methyl-1H-pyrazolo[4,3- | | | |
| | d]pyrimidine-5,7-diamine | | | |
| 104 | 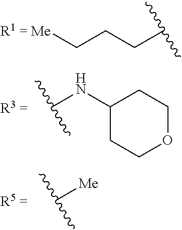 | 91.3 | 41.8 | LC/MS [M + H]⁺ = 454.2 RT (min) = 1.15 (LC/MS Method 1) LC/MS [M + H]⁺ = 454.2 RT (min) = 0.89 (LC/MS Method 2) δ 7.05 (s, 1H), 6.79 (d, J = 7.7 Hz, 1H), 6.47 (d, J = 7.7 Hz, 1H), 6.44-6.38 (m, 1H), 5.59 (s, 2H), 5.51 (s, 2H), 3.46-3.34 (m, 1H), 3.27-3.16 (m, 2H), 2.65-2.58 (m, 1H), 2.23 (s, 3H), 1.80-1.68 (m, 2H), 1.54-1.40 (m, 2H), 1.34-1.24 (m, 2H), 1.24-1.14 (m, 2H), 0.84 (t, J = 7.4 Hz, 3H) |
| | N7-butyl-1-[(2-methoxy-4-{[(oxan-4- | | | |
| | yl)amino]methyl}-phenyl)methyl]- | | | |
| | 3-methyl-1H-pyrazolo[4,3-d] | | | |
| | pyrimidine-5,7-diamine | | | |
| 105 | 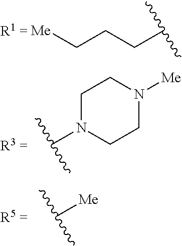 | 62.3 | 9.4 | LC/MS [M + H]⁺ = 453.2 RT (min) = 1.25 (LC/MS Method 1) LC/MS [M + H]⁺ = 453.2 RT (min) = 1.06 (LC/MS Method 2) δ 6.95 (s, 1H), 6.74 (d, J = 7.6 Hz, 1H), 6.42 (d, J = 7.7 Hz, 1H), 6.35 (t, J = 5.4 Hz, 1H), 5.64 (s, 2H), 5.52 (s, 2H), 3.83 (s, 3H), 2.33 (s, 6H), 2.24 (s, 3H), 2.15 (s, 3H), 1.50-1.39 (m, 2H), 1.23-1.09 (m, 2H), 0.82 (t, J = 7.3 Hz, 3H) |
| | N7-butyl-1-({2-methoxy-4-[(4- | | | |
| | methylpiperazin-1-yl)methyl]phenyl} | | | |
| | methyl)-3-methyl-1H-pyrazolo | | | |
| | [4,3-d]pyrimidine-5,7-diamine | | | |
| 106 | 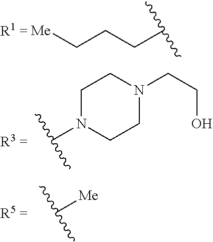 | 63.3 | 20.6 | LC/MS [M + H]⁺ = 483.2 RT (min) = 1.13 (LC/MS Method 1) LC/MS [M + H]⁺ = 483.3 RT (min) = 0.84 (LC/MS Method 2) δ 6.96 (s, 1H), 6.75 (d, J = 7.7 Hz, 1H), 6.49- 6.36 (m, 2H), 5.70 (s, 2H), 5.53 (s, 2H), 3.83 (s, 3H), 2.49-2.29 (m, 6H), 2.24 (s, 3H), 1.46 (p, J = 7.1 Hz, 2H), 1.18 (h, J = 7.3 Hz, 2H), 0.84 (t, J = 7.4Hz, 3H) |
| | 2-{4-[(4-{[5-amino-7-(butylamino)- | | | |
| | 3-methyl-1H-pyrazolo[4,3-d] | | | |
| | pyrimidin-1-yl]methyl}-3-methoxy- | | | |
| | phenyl)methyl]piperazin-1- | | | |
| | yl}ethan-1-ol | | | |
| 107 | 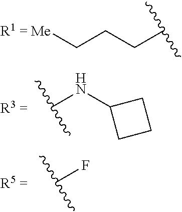 | 46.5 | 28.4 | LC/MS [M + H]⁺ = 428.3 RT (min) = 1.45 (LC/MS Method 1) LC/MS [M + H]⁺ = 428.1 RT (min) = 1.08 (LC/MS Method 2) δ 7.04 (d, J = 1.5 Hz, 1H), 6.85-6.80 (m, 1H), 6.77 (t, J = 5.6 Hz, 1H), 6.62 (d, J = 7.7 Hz, 1H), 5.85 (s, 2H), 5.46 (s, 2H), 2.11- 2.02 (m, 2H), 1.83-1.74 (m, 2H), 1.69- 1.48 (m, 3H), 1.31-1.20 (m, 2H), 0.88 (t, J = 7.4 Hz, 3H) |
| | N7-butyl-1-({4-[(cyclobutylamino) | | | |
| | methyl]-2-methoxyphenyl} | | | |
| | methyl)-3-fluoro-1H-pyrazolo[4,3- | | | |
| | d]pyrimidine-5,7-diamine | | | |
| 108 | 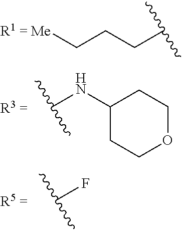 | 72.0 | 87.2 | LC/MS [M + H]⁺ = 458.3 RT (min) = 1.38 (LC/MS Method 1) LC/MS [M + H]⁺ = 458.3 RT (min) = 1.01 (LC/MS Method 2) δ 7.06 (s, 1H), 6.85 (d, J = 7.7 Hz, 1H), 6.77 (t, J = 5.5 Hz, 1H), 6.64 (d, J = 7.7 Hz, 1H), 5.85 (s, 2H), 5.45 (s, 2H), 3.90-3.70 (m, 6H), 1.79 (d, J = 12.9 Hz, 2H), 1.61-1.43 (m, 2H), 1.38-1.18 (m, 4H), 0.88 (t, J = 7.3 Hz, 3H) |
| | N7-butyl-3-fluoro-1-[(2-methoxy-4- | | | |
| | {[(oxan-4-yl)amino]methyl} | | | |
| | phenyl)-methyl]-1H-pyrazolo[4,3- | | | |
| | d]pyrimidine-5,7-diamine | | | |
| 109 | 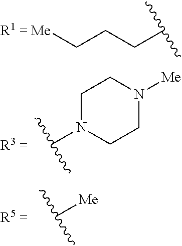 | 12.1 | 8.4 | LC/MS [M + H]⁺ = 457.1 RT (min) = 1.42 (LC/MS Method 1) LC/MS [M + H]⁺ = 457.0 RT (min) = 1.03 (LC/MS Method 2) δ 6.95 (s, 1H), 6.79 (d, J = 7.6 Hz, 1H), 6.73 (t, J = 5.6 Hz, 1H), 6.59 (d, J = 7.7 Hz, 1H), 5.87 (s, 2H), 5.46 (s, 2H), 3.80 (s, 3H), 3.45- 3.40 (m, 1H), 2.42-2.38 (m, 4H), 2.24 (s, 3H), 1.56-1.46 (m, 2H), 1.28-1.17 (m, 2H), 0.86 (t, J = 7.3 Hz, 3H) |
| | N7-butyl-3-fluoro-1-({2-methoxy- | | | |
| | 4-[(4-methyl-piperazin-1-yl)methyl]- | | | |
| | phenyl}methyl)-1H-pyrazolo[4,3-d] | | | |
| | pyrimidine-5,7-diamine | | | |
| 110 | 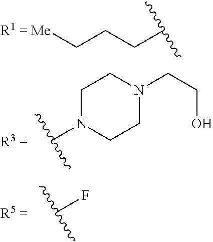 | 22.9 | 44.9 | LC/MS [M + H]⁺ = 487.3 RT (min) = 1.34 (LC/MS Method 1) LC/MS [M + H]⁺ = 487.1 RT (min) = 0.98 (LC/MS Method 2) δ 6.95 (s, 1H), 6.79 (d, J = 7.7 Hz, 1H), 6.72 (t, J = 5.5 Hz, 1H), 6.59 (d, J = 7.6 Hz, 1H), 5.85 (s, 2H), 5.45 (s, 2H), 3.79 (s, 2H), 2.50- 2.31 (m, 6H), 1.55-1.45 (m, 2H), 1.27- 1.16 (m, 2H), 0.85 (t, J = 7.4 Hz, 3H) |
| | 2-{4-[(4-{[5-amino-7-(butylamino)-3- | | | |
| | fluoro-1H-pyrazolo[4,3-d]pyrimidin-1- | | | |
| | yl]methyl}-3-methoxyphenyl)methyl]- | | | |
| | piperazin-1-yl}ethan-1-ol | | | |
| 111 | 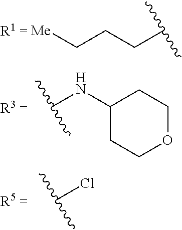 | 1,408.4 | 97.0 | LMS [M + H]⁺ 474.1 RT (min) 1.50 (UPLC Method B) δ 7.04 (s, 1H), 6.83 (br d, J = 7.3 Hz, 1H), 6.71 (br t, J = 5.0 Hz, 1H), 6.64 (d, J = 7.6 Hz, 1H), 5.88 (s, 2H), 5.54 (s, 2H), 3.85-3.74 (m, 3H), 3.59 (br s, 3H), 3.46-3.38 (m, 1H), 3.27-3.15 (m, 2H), 1.74 (br d, J = 12.5 Hz, 2H), 1.50 (quin, J = 7.2 Hz, 2H), 1.32-1.15 (m, 4H), 0.85 (t, J = 7.3 Hz, 3H) |
| | N7-butyl-3-chloro-1-[(2- | | | |
| | methoxy-4-{[(oxan-4- | | | |
| | yl)amino]methyl}phenyl)- | | | |
| | methyl]-1H-pyrazolo[4,3- | | | |
| | d]pyrimidine-5,7-diamine | | | |
| 112 | 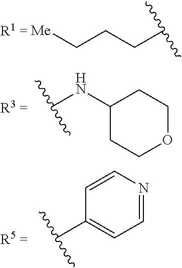 | 2,431 | 1000 | LC/MS (M + H) 517.2 RT = 1.54 min (UPLC Method B) δ 9.23-8.97 (m, 1H), 8.73 (br d, J = 5.0 Hz, 2H), 8.03 (br s, 3H), 7.29 (s, 1H), 7.25 (s, 1H), 7.19 (s, 1H), 7.08 (s, 1H), 7.05-6.92 (m, 2H), 5.91-5.75 (m, 2H), 4.16 (br s, 2H), 3.92 (br dd, J = 11.6, 3.8 Hz, 2H), 3.82 (s, 2H), 3.29 (br t, J = 11.4 Hz, 2H), 1.99 (br d, J = 10.1 Hz, 2H), 1.66-1.52 (m, 4H), 1.35-1.24 (m, 2H), 0.90 (t, J = 7.4Hz, 3H); ) |
| | N7-butyl-1-[(2-methoxy-4-{[(oxan-4- | | | |
| | yl)amino]-methyl}phenyl)methyl]-3- | | | |
| | (pyridin-4-yl)-1H-pyrazolo[4,3-d] | | | |
| | pyrimidine-5,7-diamine | | | |
| 113 | 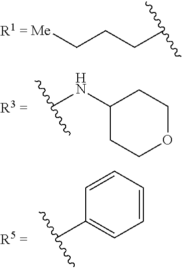 | 5,000 | 1000 | LC/MS [M + H]⁺ 516.2 RT (min) 1.71 (UPLC Method B) δ 8.35 (br d, J = 7.6 Hz, 2H), 7.42 (br t, J = 7.6 Hz, 2H), 7.34-7.21 (m, 1H), 7.08 (s, 1H), 6.81 (br d, J = 7.9 Hz, 1H), 6.56 (d, J = 7.9 Hz, 1H), 6.48 (br t, J = 5.3 Hz, 1H), 5.83 (s, 2H), 5.68 (s, 2H), 3.87 (s, 2H), 3.80 (br d, J = 11.0 Hz, 2H), 3.71 (s, 1H), 3.23 (br t, J = 11.1 Hz, 1H), 1.75 (br d, J = 11.9 Hz, 2H), 1.51 (quin, J = 7.2 Hz, 2H), 1.37-1.16 (m, 4H), 0.87 (t, J = 7.3 Hz, 3H) |
| | N7-butyl-1-[(2-methoxy-4-{[(oxan-4- | | | |
| | yl)amino]methyl}-phenyl)methyl]-3- | | | |
| | phenyl-1H-pyrazolo[4,3-d] | | | |
| | pyrimidine-5,7-diamine | | | |
| 114 | 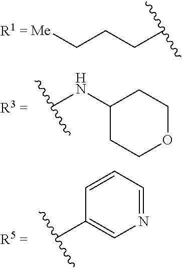 | 290.6 | 42.7 | LC/MS [M + H]⁺ 517.2 RT (min) 1.52 (UPLC Method B) δ 9.15-8.95 (m, 2H), 8.63 (br d, J = 3.7 Hz, 1H), 8.20 (br d, J = 6.7 Hz, 1H), 7.54 (dd, J = 7.8, 4.7 Hz, 1H), 7.33-7.19 (m, 1H), 7.07- 6.92 (m, 2H), 5.83 (s, 2H), 4.17 (br s, 2H), 3.92 (br dd, J = 11.3, 3.7 Hz, 2H), 3.86-3.77 (m, 2H), 3.66-3.50 (m, 1H), 3.39-3.23 (m, 1H), 2.00 (br d, J = 10.7 Hz, 2H), 1.69-1.49 (m, 4H), 1.38-1.22 (m, 2H), 0.98-0.82 (m, 3H) |
| | N7-butyl-1-[(2-methoxy-4- | | | |
| | {[(oxan-4-yl)amino]methyl}- | | | |
| | phenyl)methyl]-3-(pyridin-3- | | | |
| | yl)-1H-pyrazolo[4,3- | | | |
| | d]pyrimidine-5,7-diamine | | | |
| 115 | 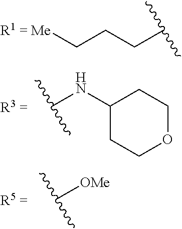 | 40.1 | 8.1 | LC/MS [M + H]⁺ = 470.5 RT (min) = 1.24 (LC/MS Method 1) LC/MS [M + H]⁺ = 470.4 RT (min) = 0.92 (LC/MS Method 2) δ 7.03 (s, 1H), 6.78 (d, J = 7.8 Hz, 1H), 6.51 (t, J = 5.5 Hz, 1H), 6.45 (d, J = 7.7 Hz, 1H), 5.59 (s, 2H), 5.39 (s, 2H), 3.89 (s, 3H), 3.83 (s, 3H), 3.44-3.30 (m, 1H), 3.28-3.17 (m, 2H), 1.80-1.66 (m, 2H), 1.55-1.42 (m, 2H), 1.31-1.13 (m, 4H), 0.85 (t, J = 7.4 Hz, 3H) |
| | N7-butyl-3-methoxy-1-[(2- | | | |
| | methoxy-4-{[(oxan-4- | | | |
| | yl)amino]methyl}phenyl)- | | | |
| | methyl]-1H-pyrazolo[4,3- | | | |
| | d]pyrimidine-5,7-diamine | | | |
| 116 | 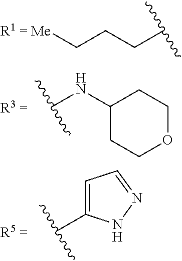 | 1,425.7 | 709.1 | LC/MS [M + H]⁺ 506.1 RT (min) 1.36 (UPLC Method B) δ 7.64-7.58 (m, 1H), 7.06 (s, 1H), 6.87 (d, J = 1.7 Hz, 1H), 6.80 (br d, J = 8.0 Hz, 1H), 6.53 (s, 1H), 6.51 (s, 1H), 5.78 (br s, 2H), 5.64 (s, 2H), 3.83 (s, 3H), 3.80 (br s, 1H), 3.72 (br s, 2H), 3.40 (br d, J = 5.0 Hz, 2H), 3.23-3.18 (m, 2H), 2.65-2.58 (m, 1H), 1.75 (br d, J = 13.8 Hz, 2H), 1.47 (quin, J = 7.3 Hz, 2H), 1.31-1.24 (m, 2H), 1.22-1.17 (m, 2H), 0.83 (t, J = 7.4 Hz, 3H) |
| | N7-butyl-1-[(2-methoxy-4- | | | |
| | {[(oxan-4-yl)amino]methyl}- | | | |
| | phenyl)methyl]-3-(1H-pyrazol- | | | |
| | 3-yl)-1H-pyrazolo[4,3- | | | |
| | d]pyrimidine-5,7-diamine | | | |
| 117 | 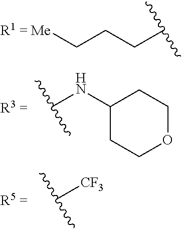 | 5,000.0 | 1000 | LC/MS [M + H]⁺ 508.1 LC RT = 1.57 min (Method 1) δ 7.09 (s, 1H), 6.87 (br d, J = 7.9 Hz, 1H), 6.74 (br t, J = 5.5 Hz, 1H), 6.67 (d, J = 7.6 Hz, 1H), 6.10 (s, 2H), 5.70 (s, 2H), 3.53-3.39 (m, 2H), 3.30-3.17 (m, 2H), 2.68-2.56 (m, 1H), 1.77 (br d, J = 11.3 Hz, 2H), 1.52 (quin, J = 7.3 Hz, 2H), 1.37-1.16 (m, 4H), 0.88 (t, J = 7.3 Hz, 3H) |
| | N7-butyl-1-[(2-methoxy-4- | | | |
| | {[(oxan-4-yl)amino]methyl}- | | | |
| | phenyl)methyl]-3-(trifluoro- | | | |
| | methyl)-1H-pyrazolo[4,3- | | | |
| | d]pyrimidine-5,7-diamine | | | |
| 118 | 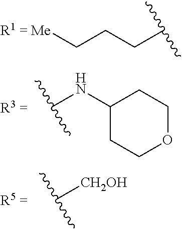 | 593.8 | 252.2 | LC/MS [M + H]⁺ = 470.1 RT (min) = 1.24 (LC/MS Method 1) LC/MS [M + H]⁺ = 470.1 RT (min) = 1.05 (LC/MS Method 2) δ 8.17 (s, 1H), 7.18 (s, 1H), 7.01 (d, J = 7.7 Hz, 1H), 6.91 (d, J = 7.6 Hz, 1H), 5.67 (s, 2H), 4.59 (s, 2H), 4.26-3.19 (m, 8H), 2.02-1.96 (m, 2H), 1.65-1.51 (m, 4H), 1.32-1.23 (m, 2H), 0.89 (t, J = 7.3 Hz, 3H) |
| | [5-amino-7-(butylamino)-1- | | | |
| | [(2-methoxy-4-{[(oxan-4- | | | |
| | yl)amino]methyl}phenyl)- | | | |
| | methyl]-1H-pyrazolo[4,3- | | | |
| | d]pyrimidin-3-yl]methanol | | | |
| 119 | 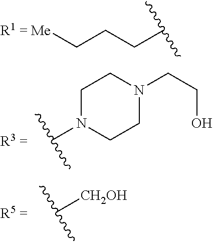 | 432.9 | 177.2 | LC/MS [M + H]⁺ = 499.2 RT (min) = 1.13 (LC/MS Method 1) LC/MS [M + H]⁺ = 499.1 RT (min) = 0.99 (LC/MS Method 2) δ 8.31 (s, 1H), 7.29 (s, 1H), 7.18 (s, 1H), 7.08 (s, 1H), 7.03 (s, 1H), 6.89 (d, J = 7.7 Hz, 1H), 6.82 (d, J = 7.7 Hz, 1H), 5.67 (s, 2H), 4.61 (s, 2H), 3.86-2.64 (m, 9H), 1.61-1.52 (m, 2H), 1.28-1.18 (m, 2H), 0.88 (t, J = 7.4 Hz, 3H) |
| | 2-{4-[(4-{[5-amino-7-(butyl- | | | |
| | amino)-3-(hydroxymethyl)- | | | |
| | 1H-pyrazolo[4,3-d]pyrimidin- | | | |
| | l-yl]methyl}-3-methoxy- | | | |
| | phenyl)methyl]piperazin-1- | | | |
| | yl}ethan-1-ol | | | |
| 120 | 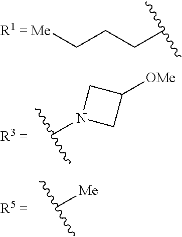 | 334.3 | 199.4 | LC/MS [M + H]⁺ = 456.4 RT (min) = 1.35 (LC/MS Method 1) LC/MS [M + H]⁺ = 456.1 RT (min) = 1.06 (LC/MS Method 2) δ 8.29 (s, 1H), 7.13 (s, 1H), 6.97 (d, J = 7.7 Hz, 1H), 6.86 (d, J = 7.6 Hz, 1H), 5.67 (s, 2H), 4.61 (s, 2H), 4.42-3.68 (m, 5H), 3.23 (s, 3H), 1.62-1.48 (m, 2H), 1.32-1.15 (m, 2H), 0.87 (t, J = 7.4 Hz, 3H) |
| | [5-amino-7-(butylamino)-1- | | | |
| | ({2-methoxy-4-[(3-methoxy- | | | |
| | azetidin-1-yl)methyl]phenyl}- | | | |
| | methyl)-1H-pyrazolo[4,3- | | | |
| | d]pyrimidin-3-yl]methanol | | | |
| 121 | 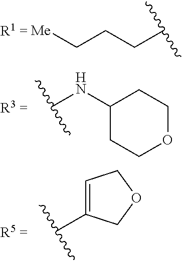 | 1,563.0 | 639.7 | LC/MS [M + H]⁺ = 507.9 RT (min) = 1.40 (LC/MS Method 1) LC/MS [M + H]⁺ = 507.9 RT (min) = 1.15 (LC/MS Method 2) δ 9.03 (s, 1H), 8.28 (s, 1H), 7.22 (d, J = 1.6 Hz, 1H), 7.01 (d, J = 7.8 Hz, 1H), 6.84 (d, J = 7.8 Hz, 1H), 6.79-6.74 (m, 1H), 5.74 (s, 2H), 4.87-4.77 (m, 4H), 4.16 (s, 2H), 3.94 (dd, J = 11.7, 4.3 Hz, 2H), 3.81 (s, 3H), 3.68- 3.19 (m, 2H), 2.00 (d, J = 12.8 Hz, 2H), 1.65-1.54 (m, 4H), 1.34-1.23 (m, 2H), 0.90 (t, J = 7.4 Hz, 3H) |
| | N7-butyl-3-(2,5-dihydro- | | | |
| | furan-3-yl)-1-[(2-methoxy-4- | | | |
| | {[(oxan-4-yl)amino]methyl}- | | | |
| | phenyl)methyl]-1H- | | | |
| | pyrazolo[4,3-d]pyrimidine- | | | |
| | 5,7-diamine | | | |
| 122 | 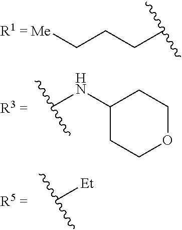 | 203.0 | 88.7 | LC/MS [M + H]⁺ = 468.2 RT (min) = 1.33 (LC/MS Method 1) LC/MS [M + H]⁺ = 468.3 RT (min) = 1.13 (LC/MS Method 2) δ 7.10 (s, 1H), 6.97 (s, 0H), 6.85-6.79 (m, 1H), 6.73 (d, J = 7.8 Hz, 0H), 6.44-6.38 (m, 2H), 6.35 (d, J = 7.6 Hz, 0H), 5.67 (s, 2H), 5.55 (s, 2H), 3.96-3.72 (m, 6H), 3.30-3.20 (m, 1H), 2.68 (q, J = 7.6 Hz, 2H), 2.14 (s, 1H), 1.81 (d, J = 12.7 Hz, 2H), 1.53-1.43 (m, 2H), 1.37-1.31 (m, 2H), 1.26-1.12 (m, 5H), 0.83 (dt, J = 14.9, 7.4 Hz, 3H) |
| | N7-butyl-3-ethyl-1-[(2- | | | |
| | methoxy-4-{[(oxan-4- | | | |
| | yl)amino]methyl}phenyl)- | | | |
| | methyl]-1H-pyrazolo[4,3- | | | |
| | d]pyrimidine-5,7-diamine | | | |
| 123 | 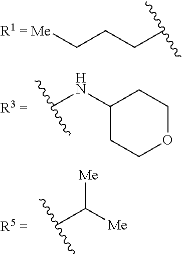 | 1,042.7 | 418.4 | LC/MS [M + H]⁺ = 482.0 RT (min) = 1.43 (LC/MS Method 1) LC/MS [M + H]⁺ = 482.1 RT (min) = 1.20 (LC/MS Method 2) δ 7.08 (s, 1H), 6.80 (d, J = 7.8 Hz, 1H), 6.35 (d, J = 7.6 Hz, 1H), 5.61 (s, 1H), 5.54 (s, 2H), 3.97-2.83 (m, 16H), 1.80 (d, J = 12.7 Hz, 2H), 1.48-1.41 (m, 2H), 1.38-1.29 (m, 2H), 1.27 (dd, J = 6.9, 1.9 Hz, 6H), 1.22- 1.13 (m, 2H), 0.83 (t, J = 7.5 Hz, 3H). |
| | N7-butyl-1-[(2-methoxy-4- | | | |
| | {[(oxan-4-yl)amino]methyl}- | | | |
| | phenyl)methyl]-3-(propan-2- | | | |
| | yl)-1H-pyrazolo[4,3- | | | |
| | d]pyrimidine-5,7-diamine | | | |
| 124 | 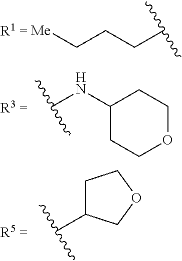 | 798.1 | 338.9 | LC/MS [M + H]⁺ = 509.9 RT (min) = 1.32 (LC/MS Method 1) LC/MS [M + H]⁺ = 510.1 RT (min) = 1.10 (LC/MS Method 2) δ 7.06 (s, 1H), 6.80 (d, J = 7.8 Hz, 1H), 6.41 (d, J = 7.4 Hz, 2H), 5.61 (s, 2H), 5.54 (s, 2H), 4.01 (t, J = 7.8 Hz, 1H), 3.93-3.76 (m, 4H), 3.44-3.12 (m, 4H), 2.69-2.65 (m, 1H), 2.27-2.16 (m, 1H), 1.78 (d, J = 12.5 Hz, 2H), 1.45 (p, J = 7.4 Hz, 2H), 1.30 (dt, J = 15.0, 8.0 Hz, 2H), 1.17 (q, J = 7.5 Hz, 2H), 0.86-0.79 (m, 3H) |
| | N7-butyl-1-[(2-methoxy-4- | | | |
| | {[(oxan-4-yl)amino]methyl}- | | | |
| | phenyl)methyl]-3-(oxolan-3- | | | |
| | yl)-1H-pyrazolo[4,3- | | | |
| | d]pyrimidine-5,7-diamine | | | |
| 125 | 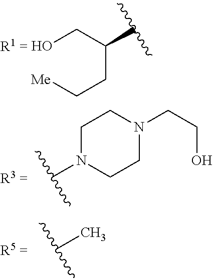 | 235.6 | 236.6 | LC/MS (M + H) 512.9 RT = 1.25 min (UPLC Method B) δ 6.98 (s, 1H), 6.89-6.73 (m, J = 7.6 Hz, 2H), 6.46 (d, J = 7.6 Hz, 2H), 5.70-5.53 (m, 2H), 5.56-5.26 (m, 1H), 4.31-4.12 (m, 1H), 3.90-3.79 (m, 2H), 3.70-3.57 (m, 1H), 3.53-3.33 (m, 4H), 2.58-2.55 (m, 3H), 2.49-2.31 (m, 7H), 2.31-2.21 (m, 3H), 2.02-1.85 (m, 3H), 1.55-1.38 (m, 1H), 1.31-1.13 (m, 2H), 1.10-0.90 (m, 2H), 0.79-0.67 (m, 3H) |
| | (2S)-2-({5-amino-1-[(4-{[4-(2- | | | |
| | hydroxyethyl)piperazin-1- | | | |
| | yl]methyl}-2-methoxy- | | | |
| | phenyl)methyl]-3-methyl-1H- | | | |
| | pyrazolo[4,3-d]pyrimidin-7- | | | |
| | yl}amino)pentan-1-ol | | | |
| 126 | 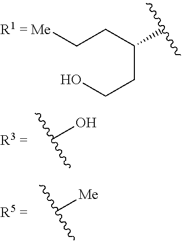 | 63.4 | 90.2 | LC/MS (M + H) 415.2 RT = 1.32 min (UPLC Method B) δ 7.03 (s, 1H), 6.79 (br d, J = 7.6 Hz, 1H), 6.50 (br d, J = 7.6 Hz, 1H), 6.07-5.85 (m, 2H), 5.72-5.57 (m, 1H), 5.56-5.44 (m, 1H), 4.47 (br d, J = 0.6 Hz, 2H), 4.41-4.23 (m, 1H), 3.93-3.75 (m, 3H), 2.27 (s, 3H), 1.76- 1.62 (m, 1H), 1.60-1.49 (m, 1H), 1.51- 1.30 (m, 2H), 1.17-1.00 (m, 2H), 0.78 (br t, J = 7.3 Hz, 3H) |
| | (3S)-3-[(5-amino-1-{[4- | | | |
| | (hydroxymethyl)-2- | | | |
| | methoxyphenyl]methyl}-3- | | | |
| | methyl-1H-pyrazolo[4,3- | | | |
| | d]pyrimidin-7- | | | |
| | yl)amino]hexan-1-ol | | | |
| 127 | 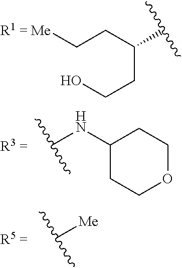 | 21.8 | 2.7 | LC/MS (M + H) 498.2 UPLC RT = 1.16 min (UPLC Method B) δ 7.10 (s, 1H), 6.82 (br d, J = 7.6 Hz, 1H), 6.43 (d, J = 7.6 Hz, 1H), 5.70 (br d, J = 8.9 Hz, 1H), 5.66 (s, 1H), 5.61-5.46 (m, 2H), 4.39-4.29 (m, 1H), 3.87 (s, 2H), 3.85-3.80 (m, 2H), 3.76 (s, 1H), 3.32 (br t, J = 6.4 Hz, 1H), 3.29- 3.19 (m, 1H), 2.73-2.62 (m, 1H), 2.26 (s, 3H), 1.93 (s, 3H), 1.79 (br d, J = 12.8 Hz, 2H), 1.71-1.59 (m, 1H), 1.57-1.47 (m, 1H), 1.47-1.25 (m, 4H), 1.15-1.03 (m, 2H), 0.79 (t, J = 7.3 Hz, 3H) |
| | (3S)-3-({5-amino-1-[(2- | | | |
| | methoxy-4-{[(oxan-4- | | | |
| | yl)amino]methyl}phenyl)- | | | |
| | methyl]-3-methyl-1H- | | | |
| | pyrazolo[4,3-d]pyrimidin-7- | | | |
| | yl}amino)hexan-1-ol | | | |
| 128 | 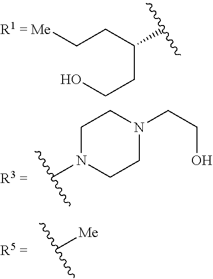 | 7.2 | 1.2 | LC/MS [M + H]⁺ 527.2 RT (min) 1.35 (UPLC Method B) δ 6.98 (s, 1H), 6.75 (br d, J = 7.9 Hz, 1H), 6.38 (d, J = 7.6 Hz, 1H), 5.67 (br d, J = 8.5 Hz, 1H), 5.61 (br s, 1H), 5.64-5.23 (m, 2H), 4.30 (dt, J = 8.7, 4.5 Hz, 1H), 3.84 (s, 1H), 3.74-3.60 (m, 1H), 3.48 (br t, J = 6.3 Hz, 1H), 3.40 (s, 1H), 3.36-3.25 (m, 2H), 2.50-2.31 (m, 6H), 2.26 (s, 3H), 1.91 (s, 2H), 1.69-1.58 (m, 1H), 1.55-1.44 (m, 1H), 1.44-1.26 (m, 2H), 1.09-0.95 (m, 2H), 0.74 (t, J = 7.3 Hz, 3H) |
| | (3S)-3-({5-amino-1-[(4-{[4-(2- | | | |
| | hydroxyethyl)piperazin-1- | | | |
| | yl]methyl}-2-methoxy- | | | |
| | phenyl)methyl]-3-methyl-1H- | | | |
| | pyrazolo[4,3-d]pyrimidin-7- | | | |
| | yl}amino)hexan-1-ol | | | |
| 129 | 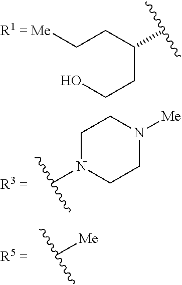 | 5.93 | 3.5 | LC/MS [M + H]⁺ 497.0 RT (min) 1.28 (UPLC Method B) δ 6.98 (s, 1H), 6.76 (br d, J = 7.6 Hz, 1H), 6.39 (br d, J = 7.3 Hz, 1H), 5.74-5.66 (m, 2H), 5.64-5.42 (m, 2H), 4.41-4.25 (m, 1H), 3.85 (s, 2H), 3.49-3.10 (m, 3H), 2.27 (s, 3H), 2.25 (br s, 3H), 1.92 (s, 2H), 1.69-1.57 (m, 2H), 1.57-1.47 (m, 1H), 1.45-1.28 (m, 3H), 1.11-1.00 (m, 2H), 0.95 (brt, J = 7.3 Hz, 1H), 0.76 (br t, J = 7.3 Hz, 3H) |
| | (3S)-3-{[5-amino-1-({2- | | | |
| | methoxy-4-[(4-methyl- | | | |
| | piperazin-1-yl)methyl]- | | | |
| | phenyl}methyl)-3-methyl- | | | |
| | 1H-pyrazolo[4,3-d]pyrimidin- | | | |
| | 7-yl]amino}hexan-1-ol | | | |
| 130 | 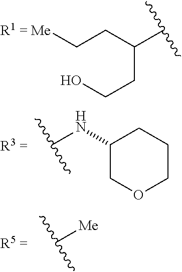 | 93.8 | 7.5 | LC/MS [M + H]⁺ 498.21 RT (min) 1.19 (UPLC Method B) δ 7.91 (br s, 2H), 7.24 (s, 1H), 6.98 (d, J = 7.9 Hz, 1H), 6.71 (br d, J = 7.9 Hz, 1H), 5.69-5.42 (m, 2H), 4.70-4.43 (m, 2H), 4.28-4.05 (m, 2H), 3.91 (br dd, J = 8.2, 3.2 Hz, 1H), 3.82 (s, 2H), 3.72- 3.57 (m, 1H), 3.27-3.03 (m, 1H), 2.30 (s, 2H), 2.15-1.92 (m, 3H), 1.83-1.68 (m, 2H), 1.57-1.41 (m, 2H), 1.37-1.16 (m, 2H), 0.83 (t, J = 7.3 Hz, 3H) |
| | (3S)-3-[(5-amino-1-{[2- | | | |
| | methoxy-4-({[(3R)-oxan-3- | | | |
| | yl]amino}methyl)phenyl]- | | | |
| | methyl}-3-methyl-1H- | | | |
| | pyrazolo[4,3-d]pyrimidin-7- | | | |
| | yl)amino]hexan-1-ol | | | |
| 131 | 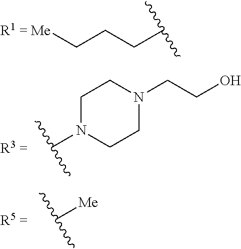 | 223.8 | 41.4 | LC/MS [M + H]⁺ 484.5 RT (min) 1.334 (Method 3) LC/MS [M + H]⁺ 484.0 RT (min) 1.230 (Method 1) δ 8.11 (s, 1H), 7.98 (s, 1H), 7.43 (d, J = 1.6 Hz, 1H), 5.96 (s, 1H), 5.60 (s, 2H), 3.89 (s, 3H), 2.55 (s, 7H), 2.49-2.43 (m, 5H), 2.21 (s, 3H), 1.61 (p, J = 7.2 Hz, 2H), 1.35 (h, J = 7.6 Hz, 2H), 0.91 (t, J = 7.4 Hz, 3H) |
| | 2-{4-[(6-{[5-amino-7-(butyl- | | | |
| | amino)-3-methyl-1H- | | | |
| | pyrazolo[4,3-d]pyrimidin-1- | | | |
| | yl]methyl}-5-methoxy- | | | |
| | pyridin-3-yl)methyl] | | | |
| | piperazin-1-yl}ethan-1-ol | | | |
| 132 | 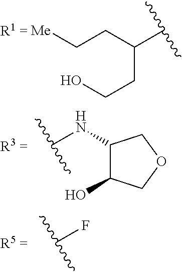 | 40.4 | 9.3 | LC/MS [M + H]⁺ 504.1 RT (min) = 1.07 (LC/MS Procedure A) δ 7.07 (s, 1H), 6.86 (br d, J = 7.6 Hz, 1H), 6.60 (d, J = 7.6 Hz, 1H), 6.12 (br d, J = 8.5 Hz, 1H), 5.91-5.78 (m, 1H), 5.57-5.36 (m, 2H), 4.45-4.30 (m, 1H), 4.14-4.03 (m, 1H), 3.88-3.83 (m, 1H), 3.89-3.78 (m, 1H), 3.82 (s, 1H), 3.59-3.43 (m, 1H), 3.40-3.32 (m, 1H), 3.14-2.98 (m, 1H), 2.56 (s, 4H), 2.52 (br s, 3H), 1.77-1.64 (m, 1H), 1.63- 1.54 (m, 1H), 1.50-1.36 (m, 2H), 1.18- 1.04 (m, 2H), 0.80 (s, 3H) |
| | (3S,4R)-4-[({4-[(5-amino-3- | | | |
| | fluoro-7-{[(3S)-1-hydroxy- | | | |
| | hexan-3-yl]amino}-1H- | | | |
| | pyrazolo[4,3-d]pyrimidin-1-yl)- | | | |
| | methyl]-3-methoxyphenyl}- | | | |
| | methyl)amino]oxolan-3-ol | | | |
| 133 | 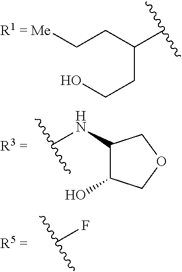 | 47.9 | 12.6 | LC/MS [M + H]⁺ = 503.9 RT (min) = 1.02 (LC/MS Procedure A) δ 7.05 (s, 1H), 6.88-6.79 (m, 1H), 6.64- 6.53 (m, 1H), 6.14-6.03 (m, 1H), 5.86 (s, 2H), 5.56-5.30 (m, 2H), 4.46-4.30 (m, 1H), 4.07-3.96 (m, 1H), 3.82 (s, 4H), 3.71-3.63 (m, 1H), 3.05-2.95 (m, 1H), 2.56 (s, 4H), 1.93 (s, 2H), 1.73-1.64 (m, 1H), 1.64-1.55 (m, 1H), 1.51-1.40 (m, 2H), 1.20-1.07 (m, 2H), 0.81 (s, 3H) |
| | (3R,4S)-4-[({4-[(5-amino-3- | | | |
| | fluoro-7-{[(3S)-1-hydroxy- | | | |
| | hexan-3-yl]amino}-1H- | | | |
| | pyrazolo[4,3-d]pyrimidin-1-yl)- | | | |
| | methyl]-3-methoxyphenyl}- | | | |
| | methyl)amino]oxolan-3-ol | | | |

Other compounds of this disclosure are shown in Table B.

TABLE B

| | | hTLR7 | hWB | Analytical Data |
|---|---|---|---|---|
| Cpd. | | Agonism | CD69 | (Mass spec, LCMS Retention Time, |
| No. | Structure | EC₅₀ (nM) | EC₅₀ (nM) | ¹H NMR (500 MHz, DMSO-d₆)) |
| 134 | 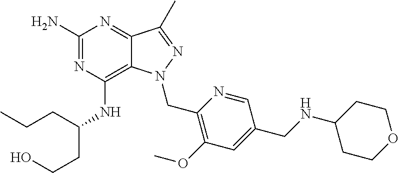 [SMILES: CCC[C@@H](CCO)Nc1nc(N)nc2c(C)nn(Cc3ncc(CNC4CCOCC4)cc3OC)c12] | | | LC/MS [M + H]⁺ = 499.3 RT (min) = 1.097 (LC/MS method D) ¹H NMR (400 MHz, DMSO-d₆) δ = 8.03 (d, J = 1.5 Hz, 1H), 7.86 (d, J = 8.6 Hz, 1H), 7.54 (s, 1H), 5.60-5.51 (m, 4H), 4.41 (br d, J = 2.2 Hz, 1H), 4.14-4.06 (m, 1H), 3.89 (s, 3H), 3.83- 3.74 (m, 4H), 3.47-3.46 (m, 2H), 3.24 (br d, J = 1.5 Hz, 2H), 2.20 (s, 3H), 1.82-1.65 (m, 4H), 1.63-1.53 (m, 2H), 1.37-1.24 (m, 4H), 0.88 (t, |
| | (3S)-3-({5-amino-1-[(3-methoxy-5-{[(oxan-4-yl)amino] | | | J = 7.3 Hz, 3H). |
| | methyl}pyridin-2-yl)methyl]-3-methyl-1H- | | | |
| | pyrazolo[4,3-d]pyrimidin-7-yl}amino)hexan-1-ol | | | |
| 135 | 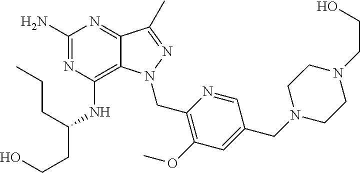 [SMILES: CCC[C@@H](CCO)Nc1nc(N)nc2c(C)nn(Cc3ncc(CN4CCN(CCO)CC4)cc3OC)c12] | 1106 | | LC/MS [M + H]⁺ = 528.3 RT (min) = 0.961 (LC/MS method D). ¹H NMR (400 MHz, DMSO-d₆) δ = 7.96 (d, J = 1.5 Hz, 1H), 7.77-7.69 (m, 1H), 7.43 (d, J = 1.3 Hz, 1H), 5.62 (br s, 2H), 5.56 (s, 2H), 4.48-4.29 (m, 2H), 3.89 (s, 3H), 3.49-3.43 (m, 6H), 2.37 (br s, 10H), 2.19 (s, 3H), 1.80- 1.63 (m, 2H), 1.61-1.49 (m, 2H), 1.37-1.20 (m, 2H), 0.86 (t, J = 7.4 Hz, 3H). |
| | (3S)-3-({5-amino-1-[(5-{[4-(2-hydroxyethyl)piperazin- | | | |
| | 1-yl]methyl}-3-methoxypyridin-2-yl)methyl]-3- | | | |
| | methyl-1H-pyrazolo[4,3-d]pyrimidin-7-yl}amino)hexan-1-ol | | | |
| 136 | 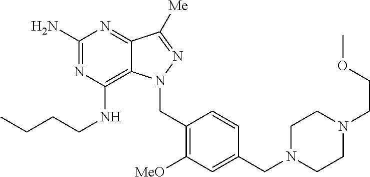 [SMILES: CCCCNc1nc(N)nc2c(C)nn(Cc3ccc(CN4CCN(CCOC)CC4)cc3OC)c12] | 163 | | [M + H]⁺ = 497.05 LC RT = 1.44 (LC/MS Method A) δ 6.96 (s, 2H), 6.75 (d, J = 7.6 Hz, 1H), 6.44 (d, J = 7.7 Hz, 2H), 5.77 (s, 2H), 5.53 (s, 2H), 3.42 (s, 1H), 3.22 (s, 2H), 2.55 (s, 6H), 2.39-2.35 (m, 6H), 2.24 (s, 4H), 1.92 (s, 1H), 1.46 (p, J = 7.1 Hz, 3H), 1.19 (p, J = 7.4 Hz, 3H), 0.84 (t, J = 7.4 Hz, 5H). |
| | N7-butyl-1-[(2-methoxy-4-{[4-(2-methoxyethyl)piperazin-1- | | | |
| | yl]methyl}phenyl)methyl]-3-methyl-1H-pyrazolo[4,3-d] | | | |
| | pyrimidine-5,7-diamine | | | |
| 137 | 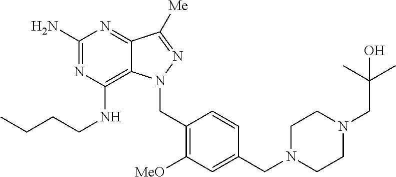 [SMILES: CCCCNc1nc(N)nc2c(C)nn(Cc3ccc(CN4CCN(CC(C)(C)O)CC4)cc3OC)c12] | 65.8 | | [M + H]⁺ = 511.01 LC RT = 1.54 (LC/MS Method A) δ 8.24 (s, 1H), 7.87 (s, 1H), 7.29 (s, 1H), 7.19 (s, 1H), 7.09 (s, 1H), 6.94- 6.89 (m, 1H), 6.77 (d, J = 7.7 Hz, 1H), 5.66 (s, 2H), 3.92 (s, 1H), 3.79 (s, 2H), 2.95 (s, 2H), 2.82 (s, 1H), 2.55 (s, 7H), 2.30 (s, 3H), 1.57 (p, J = 7.4 Hz, 2H), 1.26 (dt, J = 15.0, 7.4 Hz, 2H), 1.18 (s, 6H), 0.88 (t, J = 7.3 Hz, 3H). |
| | 1-{4-[(4-{[5-amino-7-(butylamino)-3-methyl-1H-pyrazolo[4,3-d] | | | |
| | pyrimidin-1-yl]methyl}-3-methoxyphenyl)methyl] | | | |
| | piperazin-1-yl}-2-methylpropan-2-ol | | | |

### Pharmaceutical Compositions and Administration

In another aspect, there is provided a pharmaceutical composition comprising a compound of as disclosed herein, or of a conjugate thereof, formulated together with a pharmaceutically acceptable carrier or excipient. It may optionally contain one or more additional pharmaceutically active ingredients, such as a biologic or a small molecule drug. The pharmaceutical compositions can be administered in a combination therapy with another therapeutic agent, especially an anti-cancer agent.

The pharmaceutical composition may comprise one or more excipients. Excipients that may be used include carriers, surface active agents, thickening or emulsifying agents, solid binders, dispersion or suspension aids, solubilizers, colorants, flavoring agents, coatings, disintegrating agents, lubricants, sweeteners, preservatives, isotonic agents, and combinations thereof. The selection and use of suitable excipients is taught in Gennaro, ed., Remington: The Science and Practice of Pharmacy, 20th Ed. (Lippincott Williams & Wilkins 2003).

Preferably, a pharmaceutical composition is suitable for intravenous, intramuscular, subcutaneous, parenteral, spinal or epidermal administration (e.g., by injection or infusion). Depending on the route of administration, the active compound may be coated in a material to protect it from the action of acids and other natural conditions that may inactivate it. The phrase “parenteral administration” means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, the pharmaceutical composition can be administered via a non-parenteral route, such as a topical, epidermal or mucosal route of administration, for example, intranasally, orally, vaginally, rectally, sublingually or topically.

Pharmaceutical compositions can be in the form of sterile aqueous solutions or dispersions. They can also be formulated in a microemulsion, liposome, or other ordered structure suitable to achieve high drug concentration. The compositions can also be provided in the form of lyophilates, for reconstitution in water prior to administration.

The amount of active ingredient which can be combined with a carrier material to produce a single dosage form will vary depending upon the subject being treated and the particular mode of administration and will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide a therapeutic response. For example, a single bolus may be administered, several divided doses may be administered over time, or the dose may be proportionally reduced or increased as indicated by the exigencies of the situation. It is especially advantageous to formulate parenteral compositions in dosage unit form for ease of administration and uniformity of dosage. “Dosage unit form” refers to physically discrete units suited as unitary dosages for the subjects to be treated; each unit containing a predetermined quantity of active compound calculated to produce the desired therapeutic response, in association with the required pharmaceutical carrier.

The dosage ranges from about 0.0001 to 100 mg/kg, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg, or alternatively 0.1 to 5 mg/kg. Exemplary treatment regimens are administration once per week, once every two weeks, once every three weeks, once every four weeks, once a month, once every 3 months, or once every three to 6 months. Preferred dosage regimens include 1 mg/kg body weight or 3 mg/kg body weight via intravenous administration, using one of the following dosing schedules: (i) every four weeks for six dosages, then every three months; (ii) every three weeks; (iii) 3 mg/kg body weight once followed by 1 mg/kg body weight every three weeks. In some methods, dosage is adjusted to achieve a plasma antibody concentration of about 1-1000 μg/mL and in some methods about 25-300 μg/mL.

A “therapeutically effective amount” of a compound of the invention preferably results in a decrease in severity of disease symptoms, an increase in frequency and duration of disease symptom-free periods, or a prevention of impairment or disability due to the disease affliction. For example, for the treatment of tumor-bearing subjects, a “therapeutically effective amount” preferably inhibits tumor growth by at least about 20%, more preferably by at least about 40%, even more preferably by at least about 60%, and still more preferably by at least about 80% relative to untreated subjects. A therapeutically effective amount of a therapeutic compound can decrease tumor size, or otherwise ameliorate symptoms in a subject, which is typically a human but can be another mammal. Where two or more therapeutic agents are administered in a combination treatment, “therapeutically effective amount” refers to the efficacy of the combination as a whole, and not each agent individually.

The pharmaceutical composition can be a controlled or sustained release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. See, e.g., Sustained and Controlled Release Drug Delivery Systems, J. R. Robinson, ed., Marcel Dekker, Inc., New York, 1978.

Therapeutic compositions can be administered via medical devices such as (1) needleless hypodermic injection devices; (2) micro-infusion pumps; (3) transdermal devices; (4) infusion devices; and (5) osmotic devices.

In certain embodiments, the pharmaceutical composition can be formulated to ensure proper distribution in vivo. For example, to ensure that the therapeutic compounds of the invention cross the blood-brain barrier, they can be formulated in liposomes, which may additionally comprise targeting moieties to enhance selective transport to specific cells or organs.

### Industrial Applicability and Uses

TLR7 agonist compounds disclosed herein can be used for the treatment of a disease or condition that can be ameliorated by activation of TLR7.

In one embodiment, the TLR7 agonist is used in combination with an anti-cancer immunotherapy agent—also known as an immuno-oncology agent. An anti-cancer immunotherapy agent works by stimulating a body's immune system to attack and destroy cancer cells, especially through the activation of T cells. The immune system has numerous checkpoint (regulatory) molecules, to help maintain a balance between its attacking legitimate target cells and preventing it from attacking healthy, normal cells. Some are stimulators (up-regulators), meaning that their engagement promotes T cell activation and enhances the immune response. Others are inhibitors (down-regulators or brakes), meaning that their engagement inhibits T cell activation and abates the immune response. Binding of an agonistic immunotherapy agent to a stimulatory checkpoint molecule can lead to the latter's activation and an enhanced immune response against cancer cells. Reciprocally, binding of an antagonistic immunotherapy agent to an inhibitory checkpoint molecule can prevent down-regulation of the immune system by the latter and help maintain a vigorous response against cancer cells. Examples of stimulatory checkpoint molecules are B7-1, B7-2, CD28, 4-1BB (CD137), 4-1BBL, ICOS, CD40, ICOS-L, OX40, OX40L, GITR, GITRL, CD70, CD27, CD40, DR3 and CD28H. Examples of inhibitory checkpoint molecules are CTLA-4, PD-1, PD-L1, PD-L2, LAG-3, TIM-3, Galectin 9, CEACAM-1, BTLA, CD69, Galectin-1, CD113, GPR56, VISTA, 2B4, CD48, GARP, PD1H, LAIR1, TIM-1, CD96 and TIM-4.

Whichever the mode of action of an anti-cancer immunotherapy agent, its effectiveness can be increased by a general up-regulation of the immune system, such as by the activation of TLR7. Thus, in one embodiment, this specification provides a method of treating a cancer, comprising administering to a patient suffering from such cancer a therapeutically effective combination of an anti-cancer immunotherapy agent and a TLR7 agonist as disclosed herein. The timing of administration can be simultaneous, sequential, or alternating. The mode of administration can systemic or local. The TLR7 agonist can be delivered in a targeted manner, via a conjugate.

Cancers that could be treated by a combination treatment as described above include acute myeloid leukemia, adrenocortical carcinoma, Kaposi sarcoma, lymphoma, anal cancer, appendix cancer, teratoid/rhabdoid tumor, basal cell carcinoma, bile duct cancer, bladder cancer, bone cancer, brain cancer, breast cancer, bronchial tumor, carcinoid tumor, cardiac tumor, cervical cancer, chordoma, chronic lymphocytic leukemia, chronic myeloproliferative neoplasm, colon cancer, colorectal cancer, craniopharyngioma, bile duct cancer, endometrial cancer, ependymoma, esophageal cancer, esthesioneuroblastoma, Ewing sarcoma, eye cancer, fallopian tube cancer, gallbladder cancer, gastrointestinal carcinoid tumor, gastrointestinal stromal tumor, germ cell tumor, hairy cell leukemia, head and neck cancer, heart cancer, liver cancer, hypopharngeal cancer, pancreatic cancer, kidney cancer, laryngeal cancer, chronic myelogenous leukemia, lip and oral cavity cancer, lung cancer, melanoma, Merkel cell carcinoma, mesothelioma, mouth cancer, oral cancer, osteosarcoma, ovarian cancer, penile cancer, pharyngeal cancer, prostate cancer, rectal cancer, salivary gland cancer, skin cancer, small intestine cancer, soft tissue sarcoma, testicular cancer, throat cancer, thyroid cancer, urethral cancer, uterine cancer, vaginal cancer, and vulvar cancer.

Anti-cancer immunotherapy agents that can be used in combination therapies as disclosed herein include: AMG 557, AMP-224, atezolizumab, avelumab, BMS 936559, cemiplimab, CP-870893, dacetuzumab, durvalumab, enoblituzumab, galiximab, IMP321, ipilimumab, lucatumumab, MEDI-570, MEDI-6383, MEDI-6469, muromonab-CD3, nivolumab, pembrolizumab, pidilizumab, spartalizumab, tremelimumab, urelumab, utomilumab, varlilumab, vonlerolizumab. Table B below lists their alternative name(s) (brand name, former name, research code, or synonym) and the respective target checkpoint molecule.

TABLE B

| Immunotherapy Agent | Alternative Name(s) | Target |
|---|---|---|
| AMG 557 | | B7RP-1 (ICOSL) |
| AMP-224 | | PD-1 |
| Atezolizumab | MPDL3280A, RO5541267, | PD-L1 |
| | TECENTRIQ ® | |
| Avelumab | BAVENCIO ® | PD-L1 |
| BMS 936559 | | PD-L1 |
| Cemiplimab | LIBTAYO ® | PD-1 |
| CP-870893 | | CD40 |
| Dacetuzumab | | CD40 |
| Durvalumab | IMFINZI ® | PD-L1 |
| Enoblituzumab | MGA271 | B7-H3 |
| Galiximab | | B7-1 (CD80) |
| IMP321 | | LAG-3 |
| Ipilimumab | YERVOY ® | CTLA-4 |
| Lucatumumab | | CD40 |
| MEDI-570 | | ICOS (CD278) |
| MEDI-6383 | | OX40 |
| MEDI-6469 | | OX40 |
| Muromonab-CD3 | | CD3 |
| Nivolumab | OPDIVO ® | PD-1 |
| Pembrolizumab | KEYTRUDA ® | PD-1 |
| Pidilizumab | MDV9300 | PD-1 |
| Spartalizumab | PDR001 | PD-1 |
| Tremelimumab | Ticilimumab, CP-675, CP- | CTLA-4 |
| | 675,206 | |
| Urelumab | BMS-663513 | CD137 |
| Utomilumab | PF-05082566 | CD137 |
| Varlilumab | CDX 1127 | CD27 |
| Vonlerolizumab | RG7888, MOXR0916, | OX40 |
| | pogalizumab | |

In one embodiment of a combination treatment with a TLR7 agonist, the anti-cancer immunotherapy agent is an antagonistic anti-CTLA-4, anti-PD-1, or anti-PD-L1 antibody. The cancer can be lung cancer (including non-small cell lung cancer), pancreatic cancer, kidney cancer, head and neck cancer, lymphoma (including Hodgkin's lymphoma), skin cancer (including melanoma and Merkel skin cancer), urothelial cancer (including bladder cancer), gastric cancer, hepatocellular cancer, or colorectal cancer.

In another embodiment of a combination treatment with a TLR7 agonist, the anti-cancer immunotherapy agent is an antagonistic anti-CTLA-4 antibody, preferably ipilimumab.

In another embodiment of a combination treatment with a TLR7 agonist, the anti-cancer immunotherapy agent is an antagonistic anti-PD-1 antibody, preferably nivolumab or pembrolizumab.

The TLR7 agonists disclosed herein also are useful as vaccine adjuvants.

The practice of this invention can be further understood by reference to the following examples, which are provided by way of illustration and not of limitation.

### Analytical Procedures

### NMR

The following conditions were used for obtaining proton nuclear magnetic resonance (NMR) spectra: NMR spectra were taken in either 400 Mz or 500 Mhz Bruker instrument using either DMSO-d6 or CDCl₃ as solvent and internal standard. The crude NMR data was analyzed by using either ACD Spectrus version 2015-01 by ADC Labs or MestReNova software.

Chemical shifts are reported in parts per million (ppm) downfield from internal tetramethylsilane (TMS) or from the position of TMS inferred by the deuterated NMR solvent. Apparent multiplicities are reported as: singlet-s, doublet-d, triplet-t, quartet-q, or multiplet-m. Peaks that exhibit broadening are further denoted as br. Integrations are approximate. It should be noted that integration intensities, peak shapes, chemical shifts and coupling constants can be dependent on solvent, concentration, temperature, pH, and other factors. Further, peaks that overlap with or exchange with water or solvent peaks in the NMR spectrum may not provide reliable integration intensities. In some cases, NMR spectra may be obtained using water peak suppression, which may result in overlapping peaks not being visible or having altered shape and/or integration.

### Liquid Chromatography

The following preparative and/or analytical (LC/MS) liquid chromatography methods were used.

Method 1: Column: Waters XBridge C18, 2.1 mm×50 mm, 1.7 μm particles; Mobile Phase A: 5:95 acetonitrile:water with 10 mM NH₄OAc; Mobile Phase B: 95:5 acetonitrile:water with 10 mM NH₄OAc; Temperature: 50° C.; Gradient: 0% B to 100% B over 3 min, then a 0.50 min hold at 100% B; Flow: 1 mL/min; Detection: MS and UV (220 nm). MS (ESI), positive mode unless otherwise labeled.

Method 2: Column: Waters XBridge C18, 2.1 mm×50 mm, 1.7 μm particles; Mobile Phase A: 5:95 acetonitrile:water with 0.1% TFA; Mobile Phase B: 95:5 acetonitrile:water with 0.05% TFA; Temperature: 50° C.; Gradient: 0% B to 100% B over 3 min, then a 0.50 min hold at 100% B; Flow: 1 mL/min; Detection: MS and UV (220 nm). MS (ESI), positive mode unless otherwise labeled.

Method 3: Column: Waters XBridge C18, 2.1 mm×50 mm, 1.7 μm particles; Mobile Phase A: 5:95 acetonitrile:water with 0.1% formic acid; Mobile Phase B: 95:5 acetonitrile:water 0.1% formic acid; Temperature: 40° C.; Gradient: 0.1 min hold at 5% B, 5% B to 95% B over 2.3 min, then a 0.20 min hold at 95% B, 95% B to 5% B over 0.01 min, 0.28 min hold at 5% B. Flow: 0.6 mL/min; Detection: MS and UV (254 nm).

Method 4: Column: Waters XBridge C18, 2.1 mm×50 mm, 1.7 μm particles; Mobile Phase A: 5:95 acetonitrile:water with 10 mM tertrabutyl NH₄OAc; Mobile Phase B: 95:5 acetonitrile:water with 10 mM tetrabutyl NH₄OAc; Temperature: 40° C.; Gradient: 0.1 min hold at 5% B, 5% B to 95% B over 2 min, 0.2 min hold at 95% B, 95% B to 5% B over 0.01 min, then a 0.67 min hold at 5% B; Flow: 0.6 mL/min; Detection: MS and UV (254 nm).

Method A. Column: BEH C18 2.1×50 mm; Mobile Phase A: water with 0.05% TFA; Mobile Phase B: acetonitrile with 0.05% TFA; Temperature: 50° C.; Gradient: 2-98% B over 1.7 min; then 0.50 min hold at 98% B; Flow: 0.8 mL/min. Detection: MS and UV (220 nm).

Method B. Column: Waters XBridge C18, 2.1 mm×50 mm, 1.7 am particles; Mobile Phase A: 5:95 acetonitrile:water with 10 mM NH₄OAc; Mobile Phase B: 95:5 acetonitrile:water with 10 mM NH₄OAc; Temperature: 50° C.; Gradient: 0% B to 100% B over 3 min, then a 0.50 min hold at 100% B; Flow: 1 mL/min; Detection: MS and UV (220 nm). This method is a Ultra-Performance Liquid Chromatography (UPLC™) method.

Method C. Column: BEH C18 2.1×50 mm; Mobile Phase A: water with 0.05% TFA; Mobile Phase B: acetonitrile with 0.05% TFA; Temperature: 50° C.; Gradient: 0 to 100% B over 3.0 min; Flow: 1.0 mL/min.

Method D. Column: Xbridge BEH C18 XP (50×2.1 mm), 2.5 μm; mobile phase A: 5:95 CH₃CN:H₂O with 10 mM NH₄OAc; mobile phase B: 95:5 CH₃CN:H₂O with 10 mM NH₄OAc; temperature: 50° C.; gradient: 0-100% B over 3 minutes; flow rate: 1.1 mL/min)

### Synthesis—General Procedures

Generally, the procedures disclosed herein produce a mixture of regioisomers, alkylated at the 1H or 2H position of the pyrazolopyrimidine ring system (which are also referred to as N1 and N2 regioisomers, respectively, alluding to the nitrogen that is alkylated). For brevity, the N2 regioisomers are not shown, but it is to be understood that they are present in the initial product mixture and separated at a later time, for example by preparative HPLC.

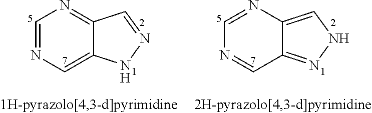 [SMILES: c1ncc2[nH]ncc2n1 ; c1ncc2n[nH]cc2n1]

The mixture of regioisomers can be separated at an early stage of the synthesis and the remaining synthetic steps carried out with the 1H regioisomer or, alternatively, the synthesis can be progressed carrying the mixture of regioisomers and separation effected at a later stage, as desired.

The compounds of the present disclosure can be prepared by a number of methods well known to one skilled in the art of synthetic organic chemistry. These methods include those described below, or variations thereof. Preferred methods include, but are not limited to, those described below in the Scheme below. The Scheme is intended to be generic, but in some instances a feature may be depicted specifically (e.g., a methyl ester, a specific protective group, or particular regioisomer) for convenience.

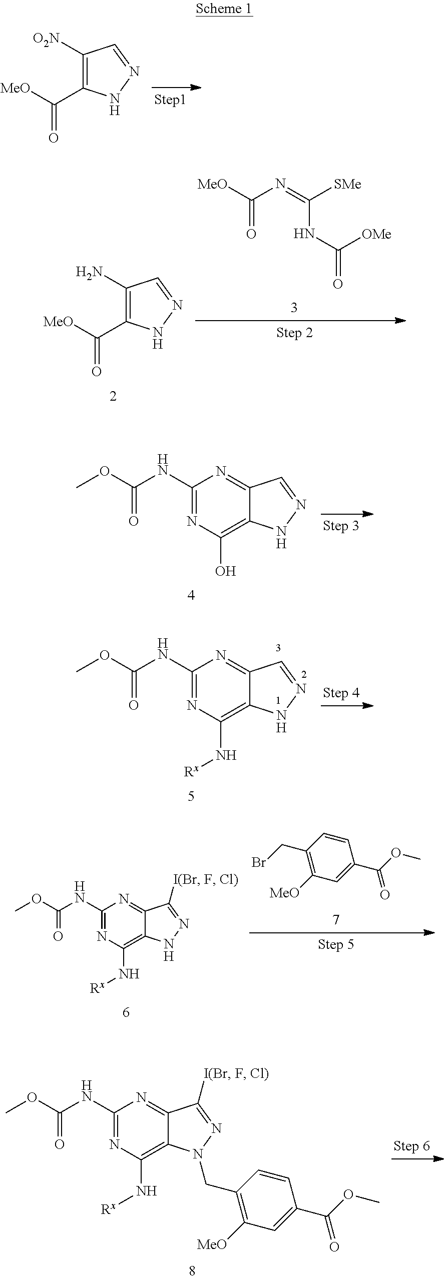

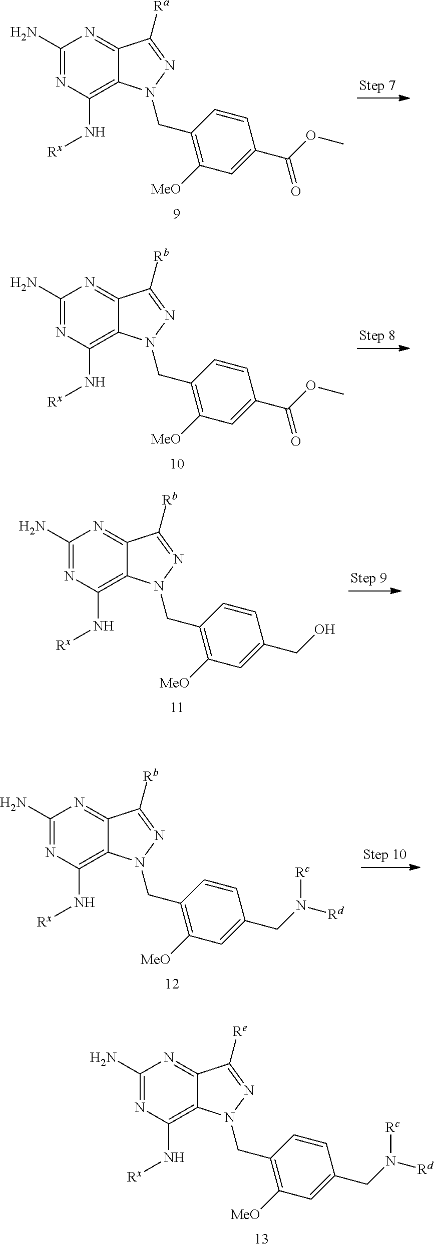

Compound 13 can be synthesized by the steps described in the above Scheme 1. In Steps 1 and 2, compound 1 is reduced under suitable conditions such as H₂ and Pd/C before reaction with compound 3 under suitable conditions (e.g., treatment with HOAc first and then with NaOMe) to form compound 4. In Step 3, compound 4 is coupled with a desired amine (e.g., n-butyl amine) using a coupling reagent such like BOP to form compound 5. (R^{x} can be an alkylamine or other suitable side chain group as described hereinabove).

In Step 4, a halogen is installed at C3 of compound 5 with a suitable halogenating reagent such as NBS, NIS, NCS or Selectfluor™. In step 5, the N1 position of compound 6 is alkylated by a suitable alkylating reagent, such as compound 7. (Some N2 alkylated product, not shown, may also be generated.) In step 6, the halogen on compound 8 can be used as a starting point for introduction of a group R^{a} at C3; for example, alkyl, alkenyl, cycloaliphatic, aromatic cycle, heteroaromaticcycle and the like. Alternatively, the three steps of halogenation, alkylation and introduction of R^{a} can be done before Step 2 (the pyrimidine formation step). R^{a} can be an end-product group or can be a precursor, subsequently modified to provide an end-product group. R^{a} can have a protective group, such as trimethylsilylethynyl, which is later removed at an appropriate stage of the synthetic process. Where R^{a} is fluoro, it can be converted into an alkoxide group with sodium alkoxide (e.g., to OMe with NaOMe). Also in step 3, the carbamate group may be deprotected or this can be done at a later stage with a suitable base, such like NaOH. The group R^{a} introduced in step 6 at C3 can be further derivatized, for example, in step 7, to generate a group R^{b}. Where R^{a} is a trimethylsilyethynyl group, it can be oxidized with RuO₂ and sodium periodate to form a carboxylic acid group, which can then be reduced to a hydroxymethyl group. In steps 8 and 9, the methyl carboxylate of compound 10 is reduced to a benzyl alcohol, which is further changed into a benzylic amine via a benzyl chloride or benzyl mesylate intermediate and displacement by an amine R^{c}R^{d}NH₂. In step 10, the group R^{b} optionally can be further elaborated to a group R^{e}, for example, by hydrogenation of a double bond or removal of a protective group to give compound 13.

### SYNTHESIS—SPECIFIC EXAMPLES

To further illustrate the foregoing, the following non-limiting, the following exemplary synthetic schemes are included. Variations of these examples within the scope of the claims are within the purview of one skilled in the art and are considered to fall within the scope of this disclosure. The reader will recognize that the skilled artisan, provided with the present disclosure and skilled in the relevant art, will be able to prepare and use the compounds disclosed herein without exhaustive examples.

Analytical data for compounds numbered 100 and higher are found in Table A or B.

### Example 1—Compound 103

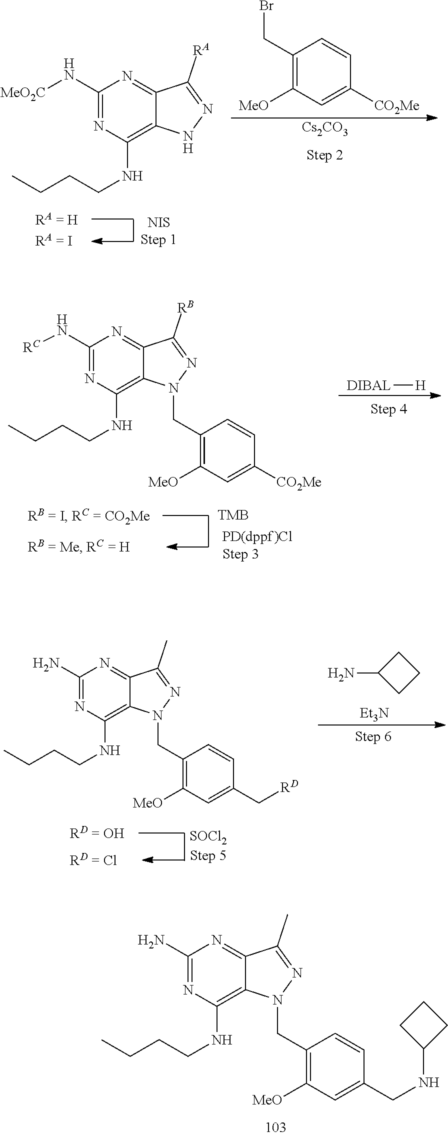

Step 1. A 100 mL flask was charged with methyl (7-(butylamino)-1H-pyrazolo[4,3-d]pyrimidin-5-yl)carbamate (4.98 g, 18.84 mmol) and DMF (60.0 mL) to form a clear solution. N-iodosuccinimide (NIS, 5.09 g, 22.61 mmol) was added in small portions at 5° C. (ice bath). The solution was stirred at 5° C. for 2 h before it was poured into 400 mL water. After filtration, the precipitate was air-dried and collected to give methyl (7-(butylamino)-3-iodo-1H-pyrazolo[4,3-d]pyrimidin-5-yl)carbamate (6.46 g, 15.73 mmol, 83% yield).

LC/MS at t=1.251 min (Method 3), MS (ESI) calcd. for [M+H]⁺ 391.0, found 391.1.

¹H NMR (400 MHz, DMSO-d6) δ 12.96 (s, 1H), 9.74 (s, 1H), 7.52 (s, 1H), 3.62 (s, 3H), 3.53 (q, J=6.5 Hz, 2H), 1.68-1.55 (m, 2H), 1.47-1.30 (m, 2H), 0.94 (t, J=7.4 Hz, 3H).

Step 2. A 100 mL flask was charged with methyl (7-(butylamino)-3-iodo-1H-pyrazolo-[4,3-d]pyrimidin-5-yl)carbamate (2.50 g, 6.41 mmol), 50.0 mL DMF, and Cs₂CO₃ (4.18 g, 12.81 mmol). After the reaction mixture was sonicated for 5 min, methyl 4-(bromomethyl)-3-methoxybenzoate (1.743 g, 6.73 mmol) in 10.0 mL DMF was added at RT. The reaction mixture was stirred at RT for 1 h before it was taken up in 200 mL DCM. The DCM solution was washed by 200 mL 10% citric acid solution (3×) and brine (30 mL). After separation, the organic phase was dried with Na₂SO₄ and filtered. The residue after evaporation of solvent was purified by chromatography (SiO₂, EtOAc:Hexane=0-100%). The fractions containing desired product were pooled and evaporated to dryness to give methyl 4-((7-(butylamino)-3-iodo-5-((methoxycarbonyl)amino)-1H-pyrazolo[4,3-d]pyrimidin-1-yl)methyl)-3-methoxybenzoate (1.41 g, 1.86 mmol, 29.1% yield).

LC/MS at t=1.738 min (Method 3). MS (ESI) calcd. 569.1, found 569.2.

¹H NMR (400 MHz, DMSO-d6) δ 9.81 (s, 1H), 7.59-7.43 (m, 2H), 7.23 (t, J=5.6 Hz, 1H), 6.72 (d, J=7.8 Hz, 1H), 5.80 (s, 2H), 3.87 (s, 3H), 3.85 (s, 3H), 3.63 (s, 3H), 3.50 (q, J=6.6 Hz, 2H), 1.54 (p, J=7.3 Hz, 2H), 1.24-1.17 (m, 2H), 0.83 (t, J=7.4 Hz, 3H).

Step 3. A 20 mL microwave vial was charged with methyl 4-((7-(butylamino)-3-iodo-5-((methoxycarbonyl)amino)-1H-pyrazolo[4,3-d]pyrimidin-1-yl)methyl)-3-methoxybenzoate (1.007 g, 1.771 mmol), 7.2 mL dioxane, [1,1′-bis(diphenylphosphino)ferrocene]dichloro-palladium(II) (0.091 g, 0.124 mmol), trimethylboroxine (TMB, 1.000 g, 7.97 mmol) and K₂CO₃ (0.734 g, 5.31 mmol). The reaction mixture was microwaved at 120° C. for 1 h. The reaction mixture was diluted with 100 mL DCM and washed sequentially with 10% citric acid (3×20 mL) and brine (30 mL). After separation, the organic phase was dried with Na₂SO₄, filtered and concentrated under reduced pressure. The final residue was purified by chromatography (SiO₂, MeOH:DCM=0-10%). The fractions with desired product were pooled and evaporated to dryness to give methyl 4-((5-amino-7-(butylamino)-3-methyl-1H-pyrazolo[4,3-d]pyrimidin-1-yl)methyl)-3-methoxybenzoate (0.212 g, 0.531 mmol, 30% yield) as a brown solid.

LC/MS at t=1.426 min (Method 4). MS (ESI) calcd. for [M−H]⁺ 397.2, found 397.2.

Step 4. A 20 mL vial charged with methyl 4-((5-amino-7-(butylamino)-3-methyl-1H-pyrazolo[4,3-d]pyrimidin-1-yl)methyl)-3-methoxybenzoate (407.2 mg, 1.022 mmol) and DCM (4 mL) was cooled to −78° C. DIBAL-H (3.07 mL, 3.07 mmol, 1 M in THF) was added dropwise. After 2 h, 0.5 mL methanol was added and the temperature was raised to RT. Potassium sodium tartrate solution (4 mL, 20%) was added and the mixture was stirred overnight and then taken up in 100 mL EtOAc. The organic phase was washed with brine (2×20 mL), dried with Na₂SO₄, filtered and evaporated under reduced pressure. The residue was purified by chromatography (SiO₂, MeOH:DCM=0-10%) to give (4-((5-amino-7-(butylamino)-3-methyl-1H-pyrazolo[4,3-d]pyrimidin-1-yl)methyl)-3-methoxyphenyl)methanol (285.0 mg, 0.769 mmol, 75% yield).

An analytical sample was purified by LC/MS with the following conditions: Column: XBridge C18, 200 mm×19 mm, 5-μm particles; Mobile Phase A: 5:95 acetonitrile:water with NH₄OAc; Mobile Phase B: 95:5 acetonitrile:water with NH₄OAc; Gradient: a 0-minute hold at 11% B, 11-51% B over 22 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The yield of the product was 20.4 mg, and its estimated purity by LCMS analysis was 99%. Analytical LC/MS was used to determine the final purity.

LC/MS Method 1: Observed Mass: 370.9; Retention Time: 1.37 min.

LC/MS Method 2: Observed Mass: 371.3; Retention Time: 1.28 min.

¹H NMR (500 MHz, DMSO-d6) δ 6.99 (s, 1H), 6.76 (d, J=7.8 Hz, 1H), 6.48 (d, J=7.7 Hz, 1H), 6.41 (t, J=5.5 Hz, 1H), 5.66 (s, 2H), 5.52 (s, 2H), 4.45 (d, J=5.5 Hz, 2H), 3.83 (d, J=1.3 Hz, 3H), 3.42 (s, OH), 2.23 (d, J=1.2 Hz, 3H), 1.54-1.44 (m, 2H), 1.27-1.15 (m, 2H), 0.89-0.82 (m, 3H).

Step 5. A 20 mL vial was charged with (4-((5-amino-7-(butylamino)-3-methyl-1H-pyrazolo[4,3-d]pyrimidin-1-yl)methyl)-3-methoxyphenyl)methanol (70.0 mg, 0.189 mmol) and DCM (3.0 mL). To the suspension was added SOCl₂ (0.034 mL, 0.472 mmol) at 0° C. After 0.5 h, the mixture was evaporated under reduced pressure and the residue (Intermediate A) was used for next step without further purification.

LC/MS at t=1.60 min (Method 3), MS (ESI) calcd. for [M+H]⁺ 389.2, found 389.2.

Step 6. A vial was charged with N7-butyl-1-(4-(chloromethyl)-2-methoxybenzyl)-3-methyl-1H-pyrazolo[4,3-d]pyrimidine-5,7-diamine (30 mg, 0.077 mmol), 0.5 mL DMF, cyclobutanamine (31.8 mg, 0.447 mmol) and Et₃N (0.022 mL, 0.154 mmol) at RT. After allowing the reaction to proceed overnight, the reaction mixture was purified by preparative HPLC to give Compound 103 (21.8 mg, 0.051 mmol, 66.7% yield). Preparative LC/MS conditions: Column: XBridge C18, 200 mm×19 mm, 5-μm particles; Mobile Phase A: 5:95 acetonitrile:water with NH₄OAc; Mobile Phase B: 95:5 acetonitrile:water with NH₄OAc; Gradient: a 0-minute hold at 10% B, 10-50% B over 23 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The yield of the product was 21.8 mg, and its estimated purity by LCMS analysis was 98%.

The following compounds were analogously prepared from Intermediate A: Compound 104: (17.2 mg, 0.037 mmol, 49.0% yield). Preparative LC/MS conditions: Column: XBridge C18, 200 mm×19 mm, 5-μm particles; Mobile Phase A: 5:95 acetonitrile:water with NH₄OAc; Mobile Phase B: 95:5 acetonitrile:water with NH₄OAc; Gradient: a 0-minute hold at 9% B, 9-49% B over 20 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation. The yield of the product was 17.2 mg, and its estimated purity by LCMS analysis was 98%.

Compound 105: (33.4 mg, 0.074 mmol, 96% yield). Preparative LC/MS conditions: Column: XBridge C18, 200 mm×30 mm, 5-μm particles; Mobile Phase A: 5:95 acetonitrile:water with NH₄OAc; Mobile Phase B: 95:5 acetonitrile:water with NH₄OAc; Gradient: a 0-minute hold at 9% B, 9-49% B over 20 minutes, then a 2-minute hold at 100% B; Flow Rate: 45 mL/min; Column Temperature: 25° C. Fraction collection was triggered by MS and UV signals. Fractions containing the desired product were combined and dried via centrifugal evaporation. The yield of the product was 33.4 mg, and its estimated purity by LCMS analysis was 100%.

Compound 106: (27.8 mg, 0.057 mmol, 74% yield). Preparative LC/MS conditions: Column: XBridge C18, 200 mm×19 mm, 5-μm particles; Mobile Phase A: 5:95 acetonitrile:water with NH₄OAc; Mobile Phase B: 95:5 acetonitrile:water with NH₄OAc; Gradient: a 0-minute hold at 8% B, 8-48% B over 23 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The yield of the product was 27.8 mg, and its estimated purity by LCMS analysis was 99%.

Compound 136 was prepared from Compound 106: A solution of Compound 106 (50 mg, 0.104 mmol) in THF (2 mL) at 0° C. was treated with NaH (9.94 mg, 0.414 mmol) and stirred for 10 min. Mel (6.48 μl, 0.104 mmol) was added and the reaction mixture was stirred at RT for 3 h. The reaction was quenched by the slow addition of MeOH. The solvent was evaporated. The crude material was purified via preparative LC/MS with the following conditions: Column: XBridge C18, 200 mm×19 mm, 5-μm particles; Mobile Phase A: 5:95 acetonitrile:water with NH₄OAc; Mobile Phase B: 95:5 acetonitrile:water with NH₄OAc; Gradient: a 0-minute hold at 10% B, 10-50% B over 20 minutes, then a 0-minute hold at 100% B; Flow Rate: 20 mL/min; Column Temperature: 25° C. Fraction collection was triggered by MS and UV signals. Fractions containing Compound 136 were combined and dried via centrifugal evaporation.

### Example 2—Compound 107

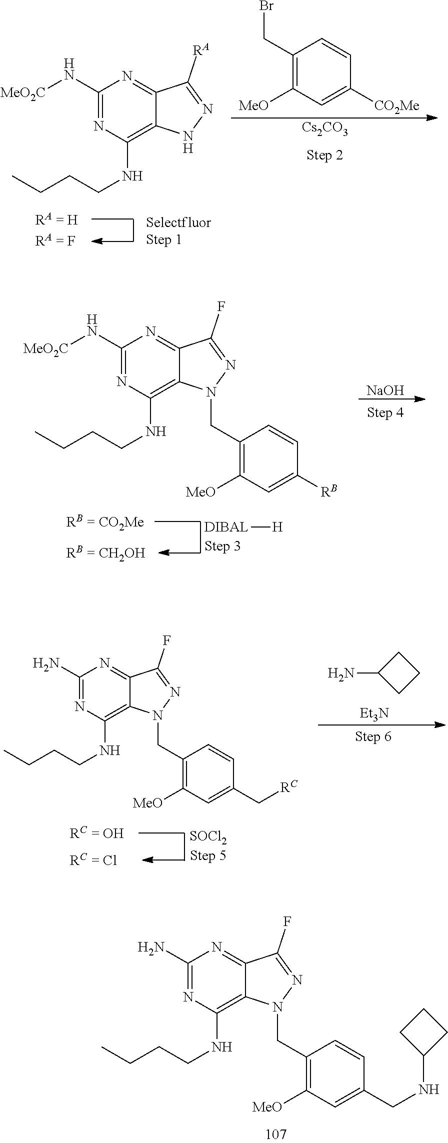

Step 1. A 30 mL vial was charged with methyl (7-(butylamino)-1H-pyrazolo[4,3-d]pyrimidin-5-yl)carbamate (972.2 mg, 3.68 mmol), 6.0 mL acetonitrile, 1.2 mL HOAc, 1-chloromethyl-4-fluoro-1,4-diazoniabicyclo[2.2.2]octane bis(tetrafluoroborate) (2606 mg, 7.36 mmol). The reaction mixture was stirred at 80° C. for 24 h. Additional acetonitrile (4.0 mL), HOAc (0.4 mL) and 1-chloromethyl-4-fluoro-1,4-diazoniabicyclo[2.2.2]octane bis(tetrafluoroborate) (5213 mg, 14.71 mmol) were added and the reaction mixture was stirred at 80° C. for another 15 h before volatiles were evaporated under reduced pressure. The residue was taken up in 60 mL DCM. The suspension was filtered and the solid was washed with more DCM (3×60 mL). The combined DCM phases were evaporated to give methyl (7-(butylamino)-3-fluoro-1H-pyrazolo[4,3-d]pyrimidin-5-yl)carbamate (886 mg, 1.569 mmol, 42.7% yield, 50% purity). The bulk of the material was used for next step without further purification. An analytical sample was purified by preparative HPLC (XBridge BEH C18 OBD Prep Column, 130 Å, 5 μm, 19 mm×150 mm, Gradient: 5%-95% acetonitrile in water (10 mM TEAA as modifier in both solvents).

LC/MS at t=1.096 min (Method 4), MS (ESI) calcd. 281.1, found 281.2.

¹H NMR (400 MHz, DMSO-d6) δ 12.10 (s, 1H), 9.73 (s, 1H), 8.41-7.12 (m, 1H), 3.63 (s, 3H), 3.56-3.48 (m, 2H), 1.71-1.53 (m, 2H), 1.50-1.31 (m, 2H), 0.94 (t, J=7.3 Hz, 3H).

Step 2. A 20 mL vial was charged with methyl (7-(butylamino)-3-fluoro-1H-pyrazolo[4,3-d]pyrimidin-5-yl)carbamate (700.0 mg, 1.24 mmol, 50% purity), 4.2 mL DMF, Cs₂CO₃ (1616 mg, 4.96 mmol) and methyl 4-(bromomethyl)-3-methoxybenzoate (3.02 mL, 1.612 mmol) at RT. After 1 h, the reaction mixture was taken up in 100 mL EtOAc. The EtOAc phase was washed with 10% citric acid solution (2×50 mL) and brine (30 mL). The organic phase was separated, dried with Na₂SO₄, filtered and evaporated under reduced pressure. The residue was purified by chromatography (SiO₂, EtOAc:Hexane=0-100%) to give methyl 4-((7-(butylamino)-3-fluoro-5-((methoxycarbonyl)amino)-1H-pyrazolo[4,3-d]pyrimidin-1-yl)methyl)-3-methoxybenzoate (300.1 mg, 0.652 mmol, 52.6% yield) as a yellow solid.

LC/MS at t=1.972 min (Method 3), MS (ESI) calcd. 461.2, found 461.2.

¹H NMR (400 MHz, DMSO-d6) δ 9.82 (s, 1H), 7.49 (d, J=7.6 Hz, 2H), 7.35 (t, J=5.6 Hz, 1H), 6.78 (d, J=7.8 Hz, 1H), 5.65 (s, 2H), 3.85 (s, 3H), 3.84 (s, 3H), 3.63 (s, 3H), 3.50 (td, J=7.0, 5.5 Hz, 2H), 1.61-1.49 (m, 2H), 1.29-1.13 (m, 2H), 0.84 (t, J=7.4 Hz, 3H).

Step 3. An oven-dried 20 mL vial was charged with methyl 4-((7-(butylamino)-3-fluoro-5-((methoxycarbonyl)amino)-1H-pyrazolo[4,3-d]pyrimidin-1-yl)methyl)-3-methoxybenzoate (0.217 g, 0.471 mmol) in 2.6 mL DCM. DIBAL-H (1.413 mL, 1.413 mmol, 1 M in THF) was added at −78° C. After 25 min, more DIBAL-H (0.5 mL, 0.5 mmol, 1.0 M in THF) was added. After another 40 min, 0.5 mL MeOH was added and the temperature was raised to RT. 4.0 mL 20% potassium sodium tartrate solution was added and the reaction mixture was stirred overnight. It was then taken up in 100 mL EtOAc. The organic phase was washed with brine (2×20 mL), dried with Na₂SO₄, filtered and evaporated under reduced pressure to give methyl (7-(butylamino)-3-fluoro-1-(4-(hydroxymethyl)-2-methoxybenzyl)-1H-pyrazolo[4,3-d]pyrimidin-5-yl)carbamate (0.204 g, 0.471 mmol, 100% yield assumed), used without further purification.

LC/MS at t=1.571 min (Method 3), MS (ESI) calcd. [M+H]⁺ 433.2, found 433.2.

Step 4. A vial was charged with methyl (7-(butylamino)-3-fluoro-1-(4-(hydroxymethyl)-2-methoxybenzyl)-1H-pyrazolo[4,3-d]pyrimidin-5-yl)carbamate (200.0 mg, 0.462 mmol) in dioxane (4.0 mL) at RT. NaOH (0.139 mL, 1.387 mmol 10.0 M) was added. The solution was stirred at 60° C. for 4 h, then cooled and acidified with dilute HCl. The precipitate was filtered and washed with water to give (4-((5-amino-7-(butylamino)-3-fluoro-1H-pyrazolo-[4,3-d]pyrimidin-1-yl)methyl)-3-methoxyphenyl)methanol (173 mg, 0.462 mmol, 100% yield assumed), which was used for next step without further purification.

LC/MS at t=1.280 min (Method 3), MS (ESI) calcd. [M+H]⁺ 375.2, found 375.2.

Step 5. A 20 mL vial was charged with (4-((5-amino-7-(butylamino)-3-fluoro-1H-pyrazolo[4,3-d]pyrimidin-1-yl)methyl)-3-methoxyphenyl)methanol (0.150 g, 0.40 mmol) and DCM (3 mL). SOCl₂ (0.058 ml, 0.800 mmol) was added at 5° C. (ice bath). After 0.5 h, the solvent was evaporated under reduced pressure to give N7-butyl-1-(4-(chloromethyl)-2-methoxybenzyl)-3-fluoro-1H-pyrazolo[4,3-d]pyrimidine-5,7-diamine (Intermediate B) (0.157 g, 0.400 mmol, 100% yield assumed), which was used for next step without further purification.

LC/MS at t=1.562 min (Method 3), MS (ESI) calcd. [M+H]⁺ 393.2, found 393.1.

Step 6. Compound 107 was obtained from Intermediate B similarly to Compound 103 from Intermediate A (2.8 mg, 0.006 mmol, 13% yield). Preparative LC/MS conditions: Column: XBridge C18, 200 mm×19 mm, 5-μm particles; Mobile Phase A: 5:95 acetonitrile:water with 10-mM NH₄OAc; Mobile Phase B: 95:5 acetonitrile:water with 10-mM NH₄OAc; Gradient: a 0-minute hold at 13% B, 13-53% B over 20 minutes, then a 0-minute hold at 100% B; Flow Rate: 20 mL/min; column Temperature: 25° C. Fraction collection was triggered by MS and UV signals. Fractions containing the desired product were combined and dried via centrifugal evaporation. The yield of the product was 2.8 mg; estimated purity by LCMS analysis 98%.

The following compounds were analogously prepared from Intermediate B:

Compound 108: (6.7 mg, 0.015 mmol, 28% yield). Preparative LC/MS conditions: Column: XBridge C18, 200 mm×19 mm, 5-μm particles; Mobile Phase A: 5:95 acetonitrile:water with 10-mM NH₄OAc; Mobile Phase B: 95:5 acetonitrile:water with 10-mM NH₄OAc; Gradient: a 0-minute hold at 11% B, 11-51% B over 24 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation. The yield of the product was 6.7 mg; estimated purity by LCMS analysis 99%.

Compound 109: (4.8 mg, 0.010 mmol, 21% yield). Preparative LC/MS conditions: Column: XBridge C18, 200 mm×19 mm, 5-μm particles; Mobile Phase A: 5:95 acetonitrile:water with 10-mM NH₄OAc; Mobile Phase B: 95:5 acetonitrile:water with 10-mM NH₄OAc; Gradient: a 0-minute hold at 11% B, 11-51% B over 24 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation. The yield of the product was 4.8 mg; estimated purity by LCMS analysis 96%.

Compound 110: (7.1 mg, 0.015 mmol, 29% yield). Preparative LC/MS conditions: Column: XBridge C18, 200 mm×19 mm, 5-μm particles; Mobile Phase A: 5:95 acetonitrile:water with 10-mM NH₄OAc; Mobile Phase B: 95:5 acetonitrile:water with 10-mM NH₄OAc; Gradient: a 0-minute hold at 9% B, 9-49% B over 24 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation. The yield of the product was 7.1 mg; estimated purity by LCMS analysis 98%.

### Example 3—Compound 115

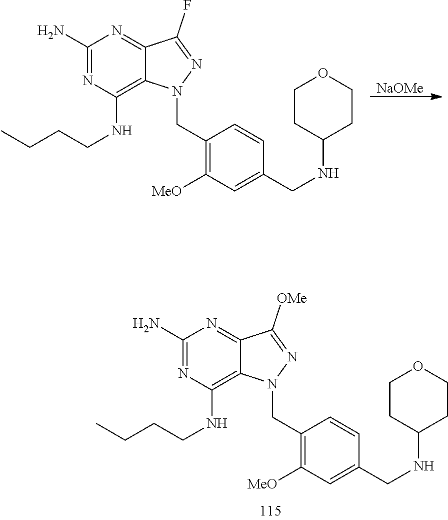 [SMILES: CCCCNc1nc(N)nc2c(F)nn(Cc3ccc(CNC4CCOCC4)cc3OC)c12 ; CCCCNc1nc(N)nc2c(OC)nn(Cc3ccc(CNC4CCOCC4)cc3OC)c12 ; "NaOMe"]

A vial was charged with N7-butyl-3-fluoro-1-(2-methoxy-4-(((tetrahydro-2H-pyran-4-yl)amino)methyl)benzyl)-1H-pyrazolo[4,3-d]pyrimidine-5,7-diamine (Compound 108) (15.0 mg, 0.033 mmol), 1.0 mL methanol and NaOMe (1 mL, 4.37 mmol, 4.37 M). After the reaction mixture was microwaved at 120° C. for 1 h, it was acidified with 1% HCl and purified by preparative HPLC to give Compound 115 (9.7 mg, 0.021 mmol, 63.0% yield). Preparative LC/MS conditions: Column: XBridge C18, 200 mm×19 mm, 5-μm particles; Mobile Phase A: 5:95 acetonitrile:water with NH₄OAc; Mobile Phase B: 95:5 acetonitrile:water with NH₄OAc; Gradient: a 0-minute hold at 12% B, 12-48% B over 20 minutes, then a 0-minute hold at 100% B; Flow Rate: 20 mL/min; Column Temperature: 25° C. Fraction collection was triggered by MS and UV signals. Fractions containing Compound 115 were combined and dried via centrifugal evaporation. The yield of the product was 9.7 mg; estimated purity by LCMS analysis 100%.

### Example 4—Compound 118

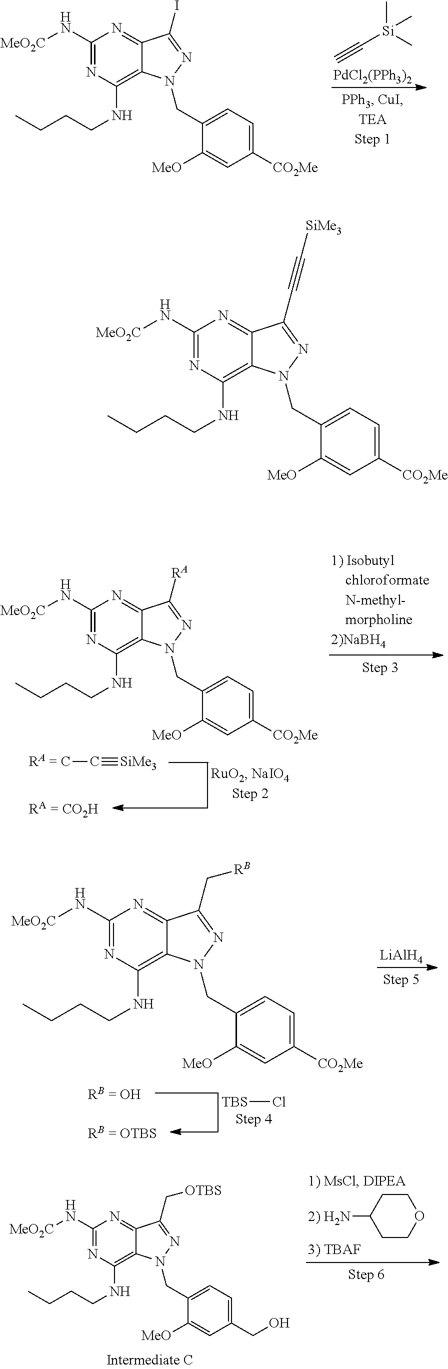

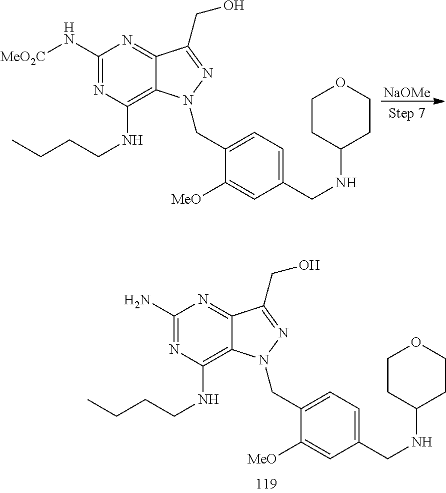 [SMILES: CCCCNc1nc(NC(=O)OC)nc2c(CO)nn(Cc3ccc(CNC4CCOCC4)cc3OC)c12 ; CCCCNc1nc(N)nc2c(CO)nn(Cc3ccc(CNC4CCOCC4)cc3OC)c12 ; "NaOMe"]

Step 1. A 20 mL microwave vial was charged with methyl 4-((7-(butylamino)-3-iodo-5-((methoxycarbonyl)amino)-1H-pyrazolo[4,3-d]pyrimidin-1-yl)methyl)-3-methoxybenzoate (1.201 g, 2.113 mmol), bis(triphenylphosphine)palladium(II) dichloride (0.148 g, 0.211 mmol), triphenylphosphine (0.194 g, 0.740 mmol), copper(I) iodide (0.089 g, 0.465 mmol), DMF (9 mL), TEA (1.5 mL) and ethynyltrimethylsilane (0.585 mL, 4.23 mmol). The reaction mixture was microwaved at 80° C. for 20 min. The cooled mixture was purified by chromatography (SiO₂, EtOAc:Hexane=0-60%) directly to give methyl 4-((7-(butylamino)-5-((methoxycarbonyl)-amino)-3-((trimethylsilyl)ethynyl)-1H-pyrazolo[4,3-d]pyrimidin-1-yl)methyl)-3-methoxybenzoate (0.933 g, 1.733 mmol, 82% yield).

LC/MS at t=2.141 min (Method 3), MS (ESI) calcd. [M+H]⁺ 539.2, found 539.4.

Step 2. A flask was charged with methyl 4-((7-(butylamino)-5-((methoxy-carbonyl)amino)-3-((trimethylsilyl)ethynyl)-1H-pyrazolo[4,3-d]pyrimidin-1-yl)methyl)-3-methoxybenzoate (2.018 g, 3.75 mmol) and 125 mL acetonitrile to form a solution at 50° C. Sodium metaperiodate (4.01 g, 18.73 mmol) was added as a solution in water (50 mL) and followed with ruthenium (IV) oxide (0.050 g, 0.375 mmol). After 1.5 h, more sodium (meta)periodate (1 g, 4.68 mmol) was added. After another 4 h, the reaction mixture was cooled down. After it was stirred at RT overnight, 3.0 mL MeOH was added and the reaction mixture was filtered through a pad of C18 silica. More acetonitrile was used to wash the silica pad. The combined filtrates were evaporated to give crude 7-(butylamino)-1-(2-methoxy-4-(methoxycarbonyl)benzyl)-5-((methoxycarbonyl)amino)-1H-pyrazolo[4,3-d]pyrimidine-3-carboxylic acid (2.065 g, 2.55 mmol, 68% yield, 60% purity), used without further purification.

LC/MS at t=1.324 min (Method 3), MS (ESI) calcd. [M+H]⁺ 487.2, found 487.4.

Step 3. A 20 mL vial was charged with 7-(butylamino)-1-(2-methoxy-4-(methoxycarbonyl)benzyl)-5-((methoxycarbonyl)amino)-1H-pyrazolo[4,3-d]pyrimidine-3-carboxylic acid (500.0 mg, 1.028 mmol) and 14 mL THF, forming a suspension. 4-methylmorpholine (0.339 mL, 3.08 mmol) and isobutyl chloroformate (0.202 mL, 1.542 mmol) were added at 5° C. After 10 min, more 4-methylmorpholine (0.339 mL, 3.08 mmol) and isobutyl chloroformate (0.202 mL, 1.542 mmol) were added. After another 10 min, sodium borohydride (234 mg, 61.6 mmol) was added and followed with 60 uL water. After 1 h, HCl was added to acidify the solution. The reaction mixture was then taken up in 100 mL DCM. The organic phase was washed with brine (2×30 mL), dried with Na₂SO₄, filtered and evaporated to give crude methyl 4-((7-(butylamino)-3-(hydroxymethyl)-5-((methoxycarbonyl)amino)-1H-pyrazolo[4,3-d]pyrimidin-1-yl)methyl)-3-methoxybenzoate (486 mg, 1.028 mmol, 100% yield assumed), which was used for next step without further purification.

LC/MS at t=1.499 min (Method 3), MS (ESI) calcd. [M+H]⁺ 473.2, found 473.4.

Step 4. A 200 mL flask was charged with methyl 4-((7-(butylamino)-3-(hydroxyl-methyl)-5-((methoxycarbonyl)amino)-1H-pyrazolo[4,3-d]pyrimidin-1-yl)methyl)-3-methoxybenzoate (939 mg, 1.988 mmol), 5.3 mL acetonitrile, imidazole (447 mg, 6.56 mmol) and TBS-Cl (599 mg, 3.98 mmol) at RT. After 15 min, MeOH (804 μl, 19.88 mmol) was added. The reaction mixture was taken up in 100 mL DCM. The organic phase was washed with diluted HCl (30 mL 1% HCl) and brine (2×30 mL), dried with Na₂SO₄, filtered and evaporated. The residue was purified by chromatography (SiO₂, MeOH:DCM=0-20%) to give methyl 4-((7-(butylamino)-3-(((tert-butyldimethylsilyl)oxy)methyl)-5-((methoxycarbonyl)amino)-1H-pyrazolo[4,3-d]pyrimidin-1-yl)methyl)-3-methoxybenzoate (424.0 mg, 0.506 mmol, 25.4% yield, 70% purity).

LC/MS at t=2.355 min (Method 3), MS (ESI) calcd. [M+H]⁺ 587.3, found 587.5.

Step 5. A 20 mL vial was charged with methyl 4-((7-(butylamino)-3-(((tert-butyldimethylsilyl)oxy)methyl)-5-((methoxycarbonyl)amino)-1H-pyrazolo[4,3-d]pyrimidin-1-yl)methyl)-3-methoxybenzoate (186.0 mg, 0.317 mmol) in 6 mL THF. LiAlH₄ (951 μl, 0.951 mmol, 1 M in THF) was added at −5° C. in three portions over 15 min. After 40 min, the reaction mixture was added to 10 mL acetone cooled at −78° C. After 10 min, 7 mL 20% Rochelle salt was added. The entire mixture was raised to RT and then taken up in 50 mL EtOAc. The organic phase was separated, washed with brine (2×30 mL), dried with Na₂SO₄, filtered and evaporated to give methyl (7-(butylamino)-3-(((tert-butyldimethylsilyl)oxy)methyl)-1-(4-(hydroxymethyl)-2-methoxybenzyl)-1H-pyrazolo[4,3-d]pyrimidin-5-yl)carbamate (Intermediate C) (63.8 mg, 0.114 mmol, 36% yield), which was used for next step without further purification.

LC/MS at t=1.932 min (Method 4), MS (ESI) calcd. [M−H]⁻ 557.3, found 557.2.

Step 6. A 4 mL vial was charged with methyl (7-(butylamino)-3-(((tert-butyldimethyl-silyl)oxy)methyl)-1-(4-(hydroxymethyl)-2-methoxybenzyl)-1H-pyrazolo[4,3-d]pyrimidin-5-yl)carbamate (38.2 mg, 0.068 mmol), 1 mL DCM, DIPEA (0.060 mL, 0.342 mmol) and mesyl-Cl (8.52 μl, 0.109 mmol) in 0.085 mL DCM at 5° C. After 20 min, tetrahydro-2H-pyran-4-amine (34.6 mg, 0.342 mmol) was added. After stirring overnight, tetra-n-butylammonium fluoride (0.205 mL, 0.205 mmol) was added. After 2 h, the reaction mixture was taken up in 50 mL EtOAc. The organic phase was washed with 10% citric acid (2×20 mL), brine (30 mL), dried with Na₂SO₄, filtered and evaporated to give methyl (7-(butylamino)-3-(hydroxymethyl)-1-(2-methoxy-4-(((tetrahydro-2H-pyran-4-yl)amino)methyl)benzyl)-1H-pyrazolo[4,3-d]pyrimidin-5-yl)carbamate (36.1 mg, 0.068 mmol, 100% yield assumed.) LC/MS at t=1.213, min (Method 4), MS (ESI) calcd. [M−H]⁻ 526.3, found 526.2.

Step 7. A 20 mL vial was charged with methyl (7-(butylamino)-3-(hydroxymethyl)-1-(2-methoxy-4-(((tetrahydro-2H-pyran-4-yl)amino)methyl)benzyl)-1H-pyrazolo[4,3-d]pyrimidin-5-yl)carbamate (0.062 g, 0.118 mmol), 2 mL MeOH and NaOH (4.72 mg, 0.118 mmol). The reaction mixture was stirred at 60° C. for 2 h before it was cooled and evaporated to dryness. Water (3 mL) was added and 4M HCl was used to neutralize the suspension. The suspension was then evaporated to dryness and the residue was purified by preparative HPLC to give Compound 118 (14.9 mg, 0.030 mmol, 27% yield). Preparative LC/MS conditions: Column: XBridge C18, 200 mm×19 mm, 5-μm particles; Mobile Phase A: 5:95 acetonitrile:water with 0.05% TFA; Mobile Phase B: 95:5 acetonitrile:water with 0.05% TFA; Gradient: a 0-minute hold at 0% B, 0-40% B over 20 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation. The yield of the product was 14.9 mg, and its estimated purity by LCMS analysis was 99%.

The following compounds were analogously prepared from Intermediate C:

Compound 119: (27.2 mg, 0.054 mmol, 54% yield). Preparative LC/MS conditions: Column: XBridge C18, 200 mm×19 mm, 5-μm particles; Mobile Phase A: 5:95 acetonitrile:water with 0.05% TFA; Mobile Phase B: 95:5 acetonitrile:water with 0.05% TFA; Gradient: a 0-minute hold at 0% B, 0-40% B over 20 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation. The yield of the product was 27.2 mg, and its estimated purity by LCMS analysis was 98%.

Compound 120: (16.7 mg, 0.035 mmol, 35% yield). Preparative LC/MS conditions: Column: XBridge C18, 200 mm×19 mm, 5-μm particles; Mobile Phase A: 5:95 acetonitrile:water with 0.05% TFA; Mobile Phase B: 95:5 acetonitrile:water with 0.05% TFA; Gradient: a 0-minute hold at 0% B, 0-40% B over 20 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation. The yield of the product was 16.7 mg, and its estimated purity by LCMS analysis was 96%.

### Example 5—Compound 123

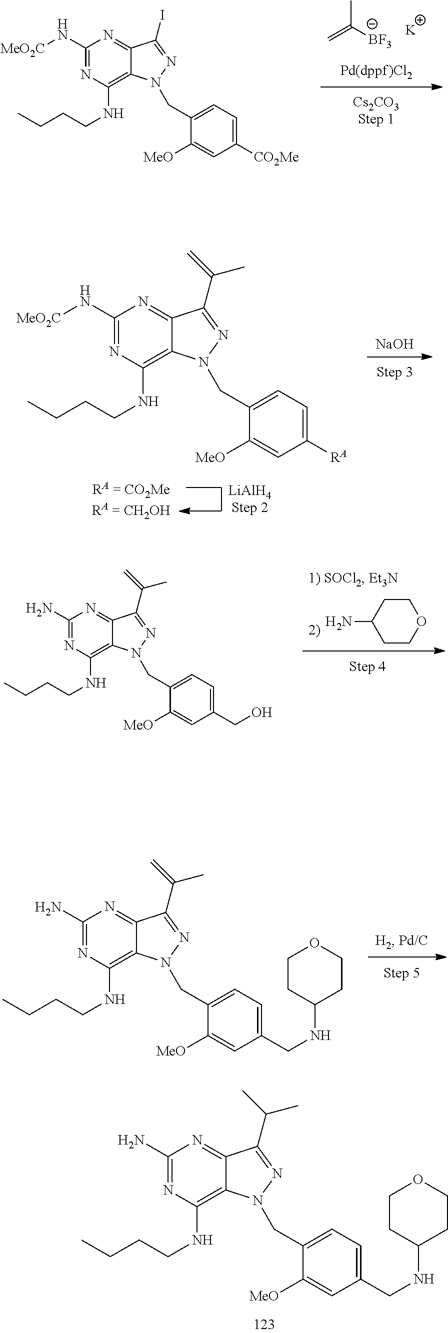

Step 1. A microwave vial was charged with methyl 4-((7-(butylamino)-3-iodo-5-((methoxycarbonyl)amino)-1H-pyrazolo[4,3-d]pyrimidin-1-yl)methyl)-3-methoxybenzoate (150 mg, 0.264 mmol), potassium trifluoro(prop-1-en-2-yl)borate (78.0 mg, 0.528 mmol), 1,1′-bis(diphenylphosphino)ferrocene-palladium(ii)dichloride dichloromethane complex (21.55 mg, 0.026 mmol), DMF (1.6 mL), water (0.400 mL) and Cs₂CO₃ (172.0 mg, 0.528 mmol) to form a suspension. The reaction mixture was microwaved at 100° C. for 1 h. 20 mL acetonitrile was added and mixture was filtered. The filtrate was evaporated down and the residue was purified by chromatography (SiO₂, EtOAc:Hexane=0-80%) to give methyl 4-((7-(butylamino)-5-((methoxycarbonyl)amino)-3-(prop-1-en-2-yl)-1H-pyrazolo[4,3-d]pyrimidin-1-yl)methyl)-3-methoxybenzoate (63.0 mg, 0.131 mmol, 49.5% yield) as a product.

LC/MS at t=2.377 min (Method 3), MS (ESI) calcd. [M+H]⁺ 483.2, found 483.3.

Step 2. A 20 mL vial was charged with methyl 4-((7-(butylamino)-5-((methoxy-carbonyl)amino)-3-(prop-1-en-2-yl)-1H-pyrazolo[4,3-d]pyrimidin-1-yl)methyl)-3-methoxybenzoate (62.1 mg, 0.129 mmol), 3.0 mL THF and LiAlH₄ (0.257 mL, 0.257 mmol, 1.0 M in THF) at 5° C. After 1 h, 200 uL acetone was added, followed by 2 mL 20% Rochelle salt. After stirring overnight, the reaction mixture was taken up in 50 mL EtOAc. The organic phase was washed with brine (2×20 mL), dried with Na₂SO₄, separated and evaporated under reduced pressure to give methyl (7-(butylamino)-1-(4-(hydroxymethyl)-2-methoxybenzyl)-3-(prop-1-en-2-yl)-1H-pyrazolo[4,3-d]pyrimidin-5-yl)carbamate (58.5 mg, 0.129 mmol, 100% yield assumed).

LC/MS at t=1.666 min (Method 3), MS (ESI) calcd. [M+H]⁺ 455.2, found 455.4.

Step 3. A 20 mL vial was charged with methyl (7-(butylamino)-1-(4-(hydroxymethyl)-2-methoxybenzyl)-3-(prop-1-en-2-yl)-1H-pyrazolo[4,3-d]pyrimidin-5-yl)carbamate (0.059 g, 0.129 mmol), MeOH (2.0 mL) and NaOH (0.20 mL, 2.000 mmol, 10 M). The suspension was stirred at 60° C. for 2 h before it was evaporated to dryness. 6.0 mL water was added and 4.0 M HCl was added to neutralize the solution. The reaction mixture was then taken up in 50 mL EtOAc. The organic phase was washed with brine (2×30 mL), dried with Na₂SO₄, filtered and evaporated to give (4-((5-amino-7-(butylamino)-3-(prop-1-en-2-yl)-1H-pyrazolo[4,3-d]pyrimidin-1-yl)methyl)-3-methoxyphenyl)methanol (0.051 g, 0.129 mmol, 100% yield assumed), which was used for next step without further purification.

LC/MS at t=1.551 min (Method 3), MS (ESI) calcd. for [M+H]⁺ 397.2, found 397.4.

Step 4. A 20 mL vial was charged with (4-((5-amino-7-(butylamino)-3-(prop-1-en-2-yl)-1H-pyrazolo[4,3-d]pyrimidin-1-yl)methyl)-3-methoxyphenyl)methanol (51.0 mg, 0.129 mmol), DCM (3.0 mL) and SOCl₂ (100 μl, 1.370 mmol) at 5° C. (ice bath). After 10 min, the reaction mixture was warmed up to RT and stirred for 1 h before it was evaporated to dryness. To the residue was added 2.0 mL DMF, tetrahydro-2H-pyran-4-amine (13.05 mg, 0.129 mmol) and DIPEA (112 μl, 0.643 mmol) at RT. The reaction mixture was stirred overnight before it was purified by reverse phase chromatography (C18, 5%-95% acetonitrile in water, 0.1% TFA as modifier in both solvents) to give N7-butyl-1-(2-methoxy-4-(((tetrahydro-2H-pyran-4-yl)amino)methyl)benzyl)-3-(prop-1-en-2-yl)-1H-pyrazolo[4,3-d]pyrimidine-5,7-diamine (60.0 mg, 0.125 mmol, 97% yield).

LC/MS at t=1.454 min (Method 4), MS (ESI) calcd. [M−H] 478.3, found 478.3.

Step 5. A nitrogen-flushed vial was charged with 20.0 mg 10% Pd/C (˜50% water), N7-butyl-1-(2-methoxy-4-(((tetrahydro-2H-pyran-4-yl)amino)methyl)benzyl)-3-(prop-1-en-2-yl)-1H-pyrazolo[4,3-d]pyrimidine-5,7-diamine (20.0 mg, 0.042 mmol) and 5.0 MeOH at RT. The flask was evacuated and refilled with hydrogen (3×). The reaction mixture was then stirred at RT under a hydrogen balloon. After 1 h, the vial was evacuated and flushed with nitrogen (3×). The reaction mixture was then filtered. The solvent was evaporated from the filtrate under reduced pressure. The residue after evaporation was purified by preparative HPLC to give Compound 123 (9.4 mg, 0.020 mmol, 46.8% yield). Preparative LC/MS conditions: Column: XBridge C18, 200 mm×19 mm, 5-μm particles; Mobile Phase A: 5:95 acetonitrile:water with NH₄OAc; Mobile Phase B: 95:5 acetonitrile:water with NH₄OAc; Gradient: a 0-minute hold at 15% B, 15-55% B over 20 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation. The yield of the product was 9.4 mg, and its estimated purity by LCMS analysis was 98%.

### Example 6—Compound 121 and Compound 124

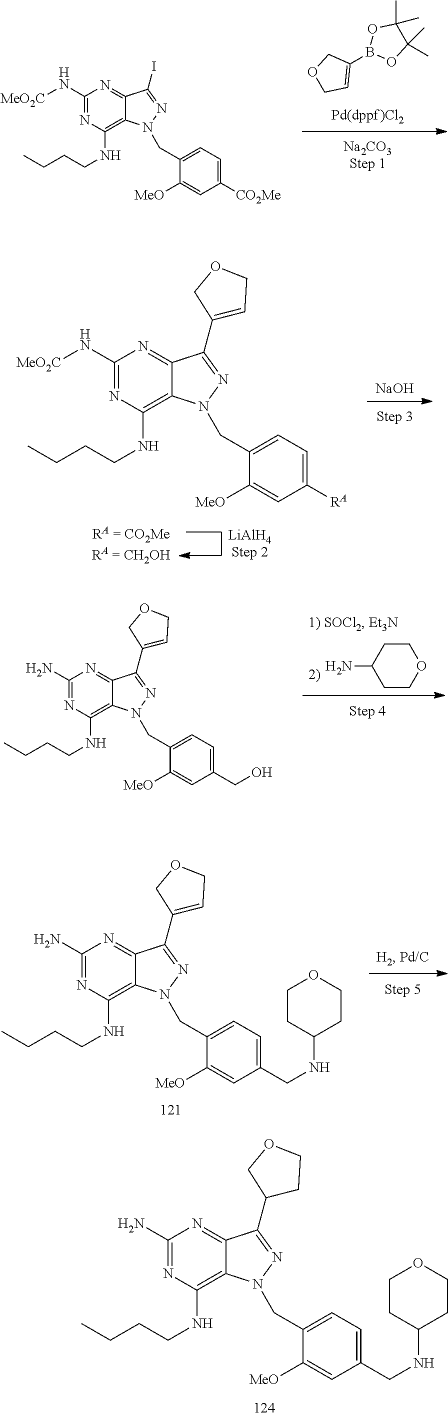

Step 1. A microwave vial was charged with methyl 4-((7-(butylamino)-3-iodo-5-((methoxycarbonyl)amino)-1H-pyrazolo[4,3-d]pyrimidin-1-yl)methyl)-3-methoxybenzoate (155.1 mg, 0.273 mmol), 1,1′-bis(diphenylphosphino)ferrocene-palladium(ii)dichloride dichloromethane complex (22.28 mg, 0.027 mmol), dioxane (3.6 mL), 2,5-dihydrofuran-3-boronic acid pinacol ester (53.5 mg, 0.273 mmol) and Na₂CO₃ (1.228 mL, 2.456 mmol). The reaction mixture was microwaved for 1 h at 100° C. and then filtered. The filtrate was evaporated under reduced pressure. The residue was purified by chromatography (SiO₂, EtOAc:Hexane=0-90%) to give methyl 4-((7-(butylamino)-3-(2,5-dihydrofuran-3-yl)-5-((methoxycarbonyl)amino)-1H-pyrazolo-[4,3-d]pyrimidin-1-yl)methyl)-3-methoxybenzoate (99.0 mg, 0.194 mmol, 71.1% yield).

LC/MS at t=1.777 min (Method 3), MS (ESI) calcd. [M+H]⁺ 511.2, found 511.4.

Step 2. A 20 mL vial was charged with methyl 4-((7-(butylamino)-3-(2,5-dihydro-furan-3-yl)-5-((methoxycarbonyl)amino)-1H-pyrazolo[4,3-d]pyrimidin-1-yl)methyl)-3-methoxybenzoate (98.0 mg, 0.192 mmol), 3 mL THF and LiAlH₄ (0.384 mL, 0.384 mmol, 1.0 M in THF) at 5° C. After 1 h, 200 uL acetone was added and followed by 2 mL 20% Rochelle salt. The reaction mixture was stirred at RT overnight before it was taken up in 50 mL EtOAc. The organic phase was washed with brine (2×30 mL), dried with Na₂SO₄, filtered and evaporated under reduced pressure to give methyl (7-(butylamino)-3-(2,5-dihydrofuran-3-yl)-1-(4-(hydroxymethyl)-2-methoxybenzyl)-1H-pyrazolo[4,3-d]pyrimidin-5-yl)carbamate (93 mg, 0.192 mmol, 100% yield assumed), which was used for next step without further purification.

LC/MS, at t=1.537 min (Method 3), MS (ESI) calcd. [M+H]⁺ 483.2, found 483.4.

Step 3. A 20 mL vial was charged with methyl (7-(butylamino)-3-(2,5-dihydrofuran-3-yl)-1-(4-(hydroxymethyl)-2-methoxybenzyl)-1H-pyrazolo[4,3-d]pyrimidin-5-yl)carbamate (0.093 g, 0.192 mmol), NaOH (0.20 mL, 2.000 mmol, 10.0 M) and MeOH (2.0 mL). After the suspension was stirred at 60° C. for 2 h, more MeOH (2 mL) and NaOH (0.20 mL, 2.0 mmol, 10.0 M NaOH) were added. The reaction mixture was stirred for another hour at 60° C. before it was cooled and neutralized with 1% HCl. The reaction mixture was then taken up in 50 mL EtOAc. The organic phase was washed with brine (2×30 mL), dried with Na₂SO₄, filtered and evaporated to give (4-((5-amino-7-(butylamino)-3-(2,5-dihydrofuran-3-yl)-1H-pyrazolo[4,3-d]pyrimidin-1-yl)methyl)-3-methoxyphenyl)methanol (0.082 g, 0.192 mmol, 100% yield assumed), which was used for next step without further purification.

LC/MS at t=1.442 min (Method 3), MS (ESI) calcd. [M+H]⁺ 425.2, found 425.4.

Step 4. A 20 mL vial was charged with (4-((5-amino-7-(butylamino)-3-(2,5-dihydrofuran-3-yl)-1H-pyrazolo[4,3-d]pyrimidin-1-yl)methyl)-3-methoxyphenyl)methanol (82 mg, 0.192 mmol), 3 mL DCM and SOCl₂ (28.0 μl, 0.384 mmol) at 5° C. (ice bath). After 20 min the reaction mixture was evaporated to dryness. To the residue was added 2.0 mL DMF, tetrahydro-2H-pyran-4-amine (97 mg, 0.960 mmol) and DIPEA (168 μl, 0.960 mmol). The reaction mixture was stirred at RT overnight before it was purified by chromatography (C18, acetonitrile:water=0-100%, 0.1% TFA in both solvents) to give Compound 121 (68.0 mg, 0.134 mmol, 69.8% yield). 20 mg of the material was future purified via preparative LC/MS with the following conditions: Column: XBridge C18, 200 mm×19 mm, 5-μm particles; Mobile Phase A: 5:95 acetonitrile:water with 0.05% TFA; Mobile Phase B: 95:5 acetonitrile:water with 0.05% TFA; Gradient: a 0-minute hold at 0% B, 0-40% B over 20 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation. The yield of the product was 14.0 mg; estimated purity by LCMS analysis 100%.

Step 5. A nitrogen flushed vial was charged with 20.0 mg 10% Pd/C (˜50% water), N7-butyl-3-(2,5-dihydrofuran-3-yl)-1-(2-methoxy-4-(((tetrahydro-2H-pyran-4-yl)amino)methyl)benzyl)-1H-pyrazolo[4,3-d]pyrimidine-5,7-diamine (22.0 mg, 0.043 mmol) and 5.0 mL MeOH at RT. The vial was evacuated and refilled with hydrogen (3×). The reaction mixture was stirred at RT overnight under a hydrogen balloon. The flask was then evacuated and refilled with nitrogen (3×). The suspension was filtered and the filtrate was evaporated to dryness under reduced pressure. The residue was purified by preparative HPLC to give Compound 124 (14.3 mg, 0.028 mmol, 64.7% yield). Preparative LC/MS conditions: Column: XBridge C18, 200 mm×19 mm, 5-μm particles; Mobile Phase A: 5:95 acetonitrile:water with NH₄OAc; Mobile Phase B: 95:5 acetonitrile:water with NH₄OAc; Gradient: a 0-minute hold at 10% B, 10-50% B over 20 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation. The yield of the product was 14.3 mg, and its estimated purity by LCMS analysis was 97%.

### Example 7—Compound 122

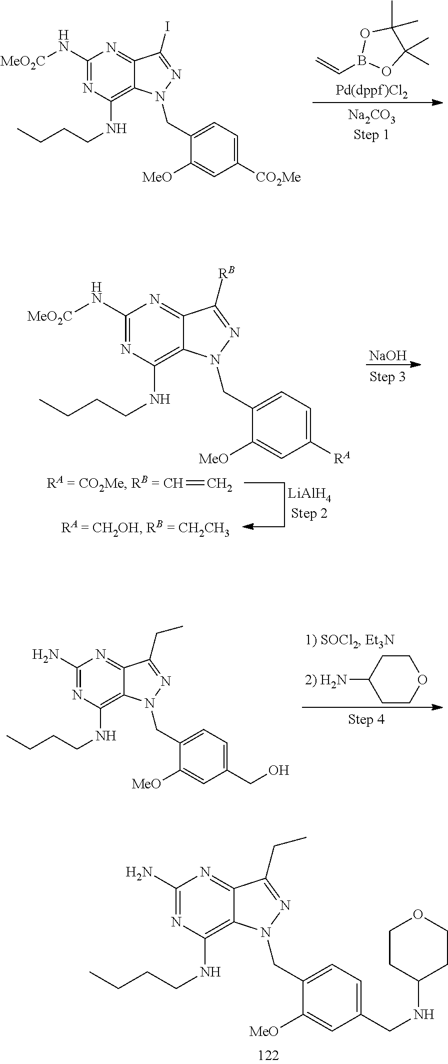

Step 1. A 20 mL microwave vial was charged with methyl 4-((7-(butylamino)-3-iodo-5-((methoxycarbonyl)amino)-1H-pyrazolo[4,3-d]pyrimidin-1-yl)methyl)-3-methoxybenzoate (255.2 mg, 0.449 mmol), [1,1′-bis(diphenylphosphino)ferrocene]dichloropalladium(II) (32.9 mg, 0.045 mmol), dioxane (8 mL), vinylboronic acid pinacol ester (135 mg, 0.880 mmol) and Na₂CO₃ (2.021 mL, 4.04 mmol, 2.0 M in water). The mixture was microwaved at 100° C. for 1 h. The reaction mixture was taken up in 100 mL EtOAc. The organic phase was washed with 10% citric acid (2×30 mL) and brine (30 mL). It was then dried with Na₂SO₄, filtered and evaporated under reduced pressure. The residue was purified by chromatography (C₁₈, Acetonitrile:water=0-100%, 0.1% TFA in both solvents as modifier) to give methyl 4-((7-(butylamino)-5-((methoxycarbonyl)amino)-3-vinyl-1H-pyrazolo[4,3-d]pyrimidin-1-yl)methyl)-3-methoxybenzoate (137.0 mg, 0.292 mmol, 65.1% yield).

LC/MS at t=1.818 min (Method 3), MS (ESI) calcd. [M+H]⁺ 469.2, found 469.2.

Step 2. A 20 mL vial was charged with methyl 4-((7-(butylamino)-5-((methoxy-carbonyl)amino)-3-vinyl-1H-pyrazolo[4,3-d]pyrimidin-1-yl)methyl)-3-methoxybenzoate (130.1 mg, 0.278 mmol), 5.0 mL THF and LiAlH₄ (555 μl, 0.555 mmol, 1.0 M in THF) at 5° C. After 1 h, 0.5 mL acetone was added, followed by 2 mL 20% Rochelle salt. The reaction mixture was stirred at RT overnight and taken up in 100 mL EtOAc. The organic phase was washed with brine (3×30 mL), dried with Na₂SO₄, filtered and evaporated under reduced pressure to give methyl (7-(butylamino)-3-ethyl-1-(4-(hydroxymethyl)-2-methoxybenzyl)-1H-pyrazolo[4,3-d]pyrimidin-5-yl)carbamate (101.2 mg, 0.229 mmol, 82% yield), used without further purification.

LC/MS at t=1.570 min (Method 3), MS (ESI) calcd. [M+H]⁺ 443.5, found 443.4.

Step 3. A 20 mL vial was charged with methyl (7-(butylamino)-3-ethyl-1-(4-(hydroxymethyl)-2-methoxybenzyl)-1H-pyrazolo[4,3-d]pyrimidin-5-yl)carbamate (101.2 mg, 0.229 mmol), 2.0 mL MeOH and NaOH (0.20 mL, 2.000 mmol, 10.0 M). The reaction mixture was stirred at 60° C. for 3 h before it was cooled and neutralized with 1% HCl. The reaction mixture was taken up in 100 mL EtOAc. The organic phase was washed with brine (2×30 mL), dried with Na₂SO₄, filtered and evaporated to give (4-((5-amino-7-(butylamino)-3-ethyl-1H-pyrazolo[4,3-d]pyrimidin-1-yl)methyl)-3-methoxyphenyl)methanol (88 mg, 0.229 mmol, 100% yield), which was used for next step without further purification.

LC/MS at t=1.454 min (Method 3), MS (ESI) calcd. [M+H]⁺ 385.2, found 385.4.

Step 4. A 20 mL vial was charged with (4-((5-amino-7-(butylamino)-3-ethyl-1H-pyrazolo[4,3-d]pyrimidin-1-yl)methyl)-3-methoxyphenyl)methanol (44.2 mg, 0.115 mmol), 3.0 mL DCM and SOCl₂ (16.79 μl, 0.230 mmol) at 5° C. (ice bath). After 0.5 h, the reaction mixture was evaporated to dryness. To the residue was added 2.0 mL DMF, tetrahydro-2H-pyran-4-amine (11.63 mg, 0.115 mmol) and DIPEA (100 μl, 0.575 mmol). The reaction mixture was stirred at 60° C. for 2 h before it was cooled and taken up in 100 mL EtOAc. The organic phase was washed with saturated NH₄OAc (2×30 mL), brine (30 mL), dried with Na₂SO₄, filtered and evaporated under reduced pressure. The residue was purified by preparative HPLC to give Compound 122 (12.5 mg, 0.027 mmol, 23.24% yield). Preparative LC/MS conditions: Column: XBridge C18, 200 mm×19 mm, 5-μm particles; Mobile Phase A: 5:95 acetonitrile:water with NH₄OAc; Mobile Phase B: 95:5 acetonitrile:water with NH₄OAc; Gradient: a 0-minute hold at 10% B, 10-50% B over 20 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation. The yield of the product was 12.5 mg, and its estimated purity by LCMS analysis was 90%.

### Example 8—Compound 101

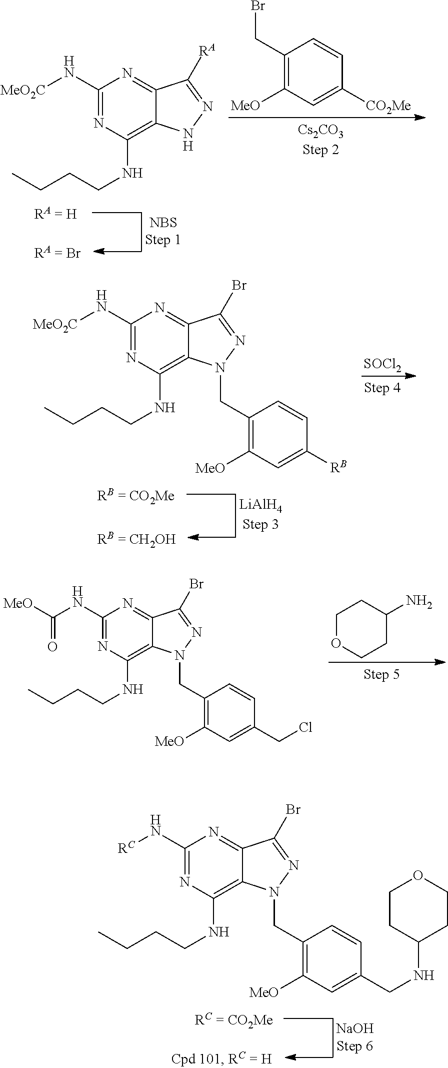

Step 1. Methyl (7-(butylamino)-1H-pyrazolo[4,3-d]pyrimidin-5-yl)carbamate (1500 mg, 5.68 mmol) was suspended in DMF (28.4 mL) at RT. N-bromosuccinimide (1212 mg, 6.81 mmol) was added in a single portion and the reaction was stirred at RT for 90 minutes. The reaction mixture was partitioned between CH₂Cl₂ and half-saturated NaHCO₃ solution. The organic phase was separated and washed with 2 additional portions of half-saturated NaHCO₃, 10% LiCl solution (1×) and dried over Na₂SO₄. The crude product was concentrated onto CELITE™ and purified by column chromatography (120 g SiO2, 0 to 8% MeOH—CH₂Cl₂) to afford methyl (3-bromo-7-(butylamino)-1H-pyrazolo[4,3-d]pyrimidin-5-yl)carbamate (1.76 g, 90%).

¹H NMR (400 MHz, DMSO-d6) δ 12.89 (br s, 1H), 9.82 (br s, 1H), 7.59 (br s, 1H), 3.67-3.60 (m, 3H), 3.60-3.46 (m, 2H), 1.72-1.56 (m, 2H), 1.40 (dq, J=14.7, 7.4 Hz, 2H), 0.94 (br t, J=7.4 Hz, 3H).

LC/MS (M+H) 343.1/345.1; LC RT=0.62 min (Method A).

Step 2. Methyl (3-bromo-7-(butylamino)-1H-pyrazolo[4,3-d]pyrimidin-5-yl)carbamate (1.76 g, 5.13 mmol) and Cs₂CO₃ (4.18 g, 12.82 mmol) were suspended in DMF at RT. Methyl 4-(bromomethyl)-3-methoxybenzoate (1.329 g, 5.13 mmol) was added and the reaction was stirred at RT. After 90 min, the reaction mixture was diluted with EtOAc (150 mL) and filtered through CELITE™. The organic layer was washed with H₂O (2×), 10% LiCl solution (1×), and brine (1×). The organic phase was dried over MgSO₄ and concentrated. The crude product containing a mixture of N1 and N2 isomers was purified by column chromatography (120 g SiO₂, 10 to 100% EtOAc-hexane gradient elution). The N1 isomer was further purified by reverse phase chromatography (C18 100 g Gold, 10 to 90% CH₃OH—H2O, with 0.05% TFA). Fractions containing the product were concentrated until all the volatiles were removed. The remaining aqueous solution was rendered basic with saturated NaHCO₃ solution and extracted with CH₂Cl₂ (3×). The combined organic phases were dried over Na₂SO₄ and concentrated to afford analytically pure methyl 4-((3-bromo-7-(butylamino)-5-((methoxycarbonyl)amino)-1H-pyrazolo[4,3-d]pyrimidin-1-yl)methyl)-3-methoxybenzoate.

¹H NMR (400 MHz, DMSO-d6) δ 9.88 (s, 1H), 7.59-7.46 (m, 2H), 7.33 (br t, J=5.4 Hz, 1H), 6.79 (d, J=7.9 Hz, 1H), 5.86-5.72 (m, 2H), 3.85 (d, J=4.9 Hz, 6H), 3.63 (s, 3H), 3.52 (q, J=6.7 Hz, 2H), 1.55 (quin, J=7.2 Hz, 2H), 1.21 (sxt, J=7.4 Hz, 2H), 0.84 (t, J=7.4 Hz, 3H).

LC/MS (M+H) 521.0/523.0; LC RT=0.82 min (Method A).

Step 3. Methyl 4-((3-bromo-7-(butylamino)-5-((methoxycarbonyl)amino)-1H-pyrazolo[4,3-d]pyrimidin-1-yl)methyl)-3-methoxybenzoate (180 mg, 0.345 mmol) was dissolved in THF (1151 μl) at RT. LiAlH₄ (1M in THF) (690 μl, 0.690 mmol) was added dropwise via slow addition. Another 1 mL of THF was added to the reaction mixture half-way through the hydride addition to help with stirring. After 20 minutes, the reactions was quenched with MeOH and saturated Rochelle's salts. EtOAc was added and the biphasic mixture was stirred for 2 hr until the layers cleared. The organic phase was removed and the aqueous phase was extracted with EtOAc (3×). The combined organics were dried over Na₂SO₄ and concentrated to afford methyl (3-bromo-7-(butylamino)-1-(4-(hydroxymethyl)-2-methoxybenzyl)-1H-pyrazolo[4,3-d]pyrimidin-5-yl)carbamate (133 mg).

¹H NMR (400 MHz, DMSO-d6) δ 9.85 (s, 1H), 7.28 (t, J=5.5 Hz, 1H), 6.99 (s, 1H), 6.86-6.81 (m, 1H), 6.79-6.74 (m, 1H), 5.67 (s, 2H), 5.19 (t, J=5.7 Hz, 1H), 4.47 (d, J=5.7 Hz, 2H), 3.76 (s, 3H), 3.63 (s, 3H), 3.58-3.50 (m, 2H), 1.58 (sxt, J=7.4 Hz, 2H), 1.33-1.21 (m, 2H), 0.89 (t, J=7.4 Hz, 3H).

LC/MS (M+H) 493.0/495.0; LC RT=0.73 min Method A).

Step 4. Methyl (3-bromo-7-(butylamino)-1-(4-(hydroxymethyl)-2-methoxybenzyl)-1H-pyrazolo[4,3-d]pyrimidin-5-yl)carbamate (113 mg, 0.229 mmol) was dissolved in THF (2290 μl) at RT. SOCl₂ (84 μl, 1.145 mmol) was added and the reaction at RT for 30 min. Concentration and drying in vacuo afforded methyl (3-bromo-7-(butylamino)-1-(4-(chloro-methyl)-2-methoxybenzyl)-1H-pyrazolo[4,3-d]pyrimidin-5-yl)carbamate (117 mg).

¹H NMR (400 MHz, DMSO-d6) δ 10.23 (br s, 1H), 7.71-7.56 (m, 1H), 7.12 (d, J=1.3 Hz, 1H), 6.97 (dd, J=7.8, 1.3 Hz, 1H), 6.80 (d, J=7.7 Hz, 1H), 5.71 (s, 2H), 4.73 (s, 2H), 3.78 (s, 3H), 3.67 (s, 3H), 3.55 (q, J=6.8 Hz, 2H), 1.59 (quin, J=7.4 Hz, 2H), 1.33-1.18 (m, 2H), 0.92-0.84 (m, 3H).

LC/MS (M+H) 511.0/513.0; LC RT=0.86 min (Method A).

Step 5. Methyl (3-bromo-7-(butylamino)-1-(4-(chloromethyl)-2-methoxybenzyl)-1H-pyrazolo[4,3-d]pyrimidin-5-yl)carbamate (115 mg, 0.225 mmol) was dissolved in DMF at RT. Tetrahydro-2H-pyran-4-amine (46.5 μl, 0.449 mmol) was added and stirred the reaction at RT. After 4 h, another equivalent of tetrahydro-2H-pyran-4-amine (46.5 μl, 0.449 mmol) was added and the reaction was stirred for an additional 3 h. The reaction mixture was partitioned between EtOAc and half-saturated bicarbonate solution. The aqueous phase was extracted into EtOAc (3×) and the combined organics were washed with 10% LiCl solution, brine and then dried over Na₂SO₄. Concentration afforded methyl (3-bromo-7-(butylamino)-1-(2-methoxy-4-(((tetrahydro-2H-pyran-4-yl)amino)methyl)benzyl)-1H-pyrazolo[4,3-d]pyrimidin-5-yl)carbamate (96 mg) which was used without further purification.

¹H NMR (400 MHz, DMSO-d₆) δ 9.85 (s, 1H), 7.27 (br t, J=5.6 Hz, 1H), 7.03 (s, 1H), 6.85 (br d, J=7.6 Hz, 1H), 6.72 (d, J=7.5 Hz, 1H), 5.66 (s, 2H), 3.85-3.74 (m, 5H), 3.72-3.67 (m, 2H), 3.63 (s, 3H), 3.58-3.48 (m, 2H), 3.29-3.18 (m, 2H), 1.75 (br d, J=13.0 Hz, 2H), 1.65-1.51 (m, 3H), 1.33-1.22 (m, 4H), 0.88 (t, J=7.3 Hz, 3H).

LC/MS (M+H) 576.1/578.0; LC RT=0.62 min (Method A).

Step 6. NaOH (10 M solution) (13.88 μl, 0.139 mmol) was added to a stirred solution of methyl (3-bromo-7-(butylamino)-1-(2-methoxy-4-(((tetrahydro-2H-pyran-4-yl)amino)methyl)benzyl)-1H-pyrazolo[4,3-d]pyrimidin-5-yl)carbamate (16 mg, 0.028 mmol) in dioxane (278 μL). The reaction mixture was heated to 80° C. After 6 h, the reaction mixture was cooled to RT, neutralized with acetic acid (7.94 μl, 0.139 mmol) and concentrated. The crude product was re-dissolved in DMF, filtered through a PTFE frit and purified via preparative LC/MS with the following conditions: Column: XBridge C18, 200 mm×19 mm, 5-μm particles; Mobile Phase A: 5:95 acetonitrile:water with NH₄OAc; Mobile Phase B: 95:5 acetonitrile:water with ammoniumacetate; Gradient: a 0-minute hold at 13% B, 13-53% B over 20 minutes, then a 4-minute hold at 100% B; Flow Rate: 20 mL/min; Column Temperature: 25° C. Fraction collection was triggered by MS signals. Fractions containing the desired product were combined and dried via centrifugal evaporation to afford Compound 101 (3.2 mg).

Compound 111 was analogously prepared.

### Example 9—Compound 102

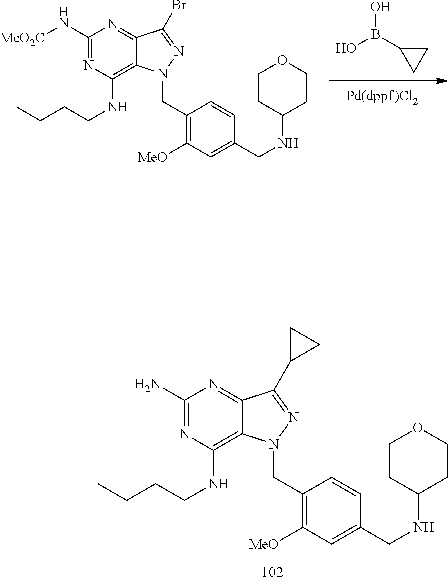 [SMILES: CCCCNc1nc(NC(=O)OC)nc2c(Br)nn(Cc3ccc(CNC4CCOCC4)cc3OC)c12 ; OB(O)C1CC1 ; CCCCNc1nc(N)nc2c(C3CC3)nn(Cc3ccc(CNC4CCOCC4)cc3OC)c12]

Step 1. Methyl (3-bromo-7-(butylamino)-1-(2-methoxy-4-(((tetrahydro-2H-pyran-4-yl)amino)methyl) benzyl)-1H-pyrazolo[4,3-d]pyrimidin-5-yl)carbamate (694 μl, 0.069 mmol) was dissolved in dioxane (694 μL) at RT. Cyclopropylboronic acid (7.15 mg, 0.083 mmol) and 2M aqueous K₃PO₄ (69.4 μl, 0.139 mmol) solution were added. The reaction mixture was sparged with N₂ for 3 min. PdCl₂(dppf)-CH₂Cl₂ adduct (5.7 mg, 6.94 μM) was introduced and N₂ was bubbled through for another 2 min. The reaction vial was sealed and heated to 120° C. in a microwave oven for 45 min. Partial reaction was observed. After cooling to RT, the reaction mixture was diluted with EtOAc, filtered through a PTFE frit and concentrated. The residue was re-subjected to the reaction conditions except with stirring at 100° C. overnight. After cooling to RT, the reaction mixture was diluted with EtOAc, filtered through a PTFE frit and concentrated. The crude material was purified via preparative LC/MS with the following conditions: Column: XBridge C18, 200 mm×19 mm, 5-μm particles; Mobile Phase A: 5:95 acetonitrile:water with 10-mM NH₄OAc; Mobile Phase B: 95:5 acetonitrile:water with 10-mM NH₄OAc; Gradient: a 0-minute hold at 15% B, 15-55% B over 20 minutes, then a 4-min hold at 100% B; Flow Rate: 20 mL/min; Column Temperature: 25° C. Fractions containing the desired product per MS signals were combined and dried via centrifugal evaporation to afford Compound 102 (3.4 mg).

### Example 10—Compound 112

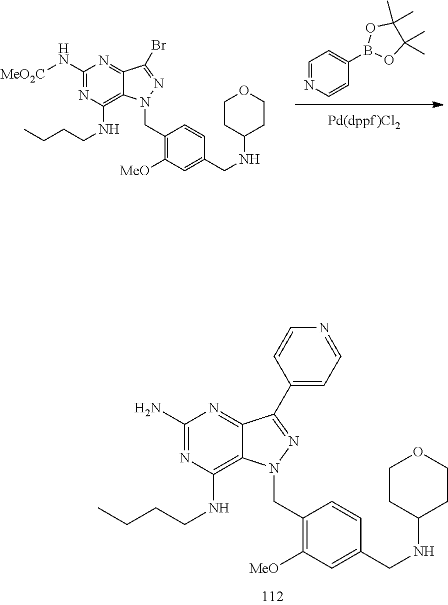 [SMILES: CCCCNc1nc(NC(=O)OC)nc2c(Br)nn(Cc3ccc(CNC4CCOCC4)cc3OC)c12 ; CC1(C)OB(c2ccncc2)OC1(C)C ; CCCCNc1nc(N)nc2c(-c3ccncc3)nn(Cc3ccc(CNC4CCOCC4)cc3OC)c12]

Step 1. Methyl (3-bromo-7-(butylamino)-1-(2-methoxy-4-(((tetrahydro-2H-pyran-4-yl)amino)methyl) benzyl)-1H-pyrazolo[4,3-d]pyrimidin-5-yl)carbamate (30 mg, 0.052 mmol) and 4-(4,4,5,5-tetramethyl-1,3,2-dioxaborolan-2-yl)pyridine (32.0 mg, 0.156 mmol) was suspended in dioxane (520 μl) at RT. Nitrogen was bubbled through the reaction mixture for 3 min. K₃PO₄ solution (2M, 26.0 μl, 0.052 mmol) was added, followed by PdCl₂(dppf)-CH₂Cl₂ adduct (4.25 mg, 5.20 μmol). Nitrogen was bubbled through the reaction mixture for an additional 2 min. The reaction vial was sealed and heated to 100° C. for 16 h. The reaction mixture was cooled to RT, diluted with EtOAc, filtered through a PTFE frit and concentrated. The residue was re-dissolved in DMF, NaOH solution (10 M, 26.0 μl, 0.260 mmol) was added. The reaction mixture was heated to 100° C. for 1 h, neutralized with AcOH (14.90 μl, 0.260 mmol), diluted with another 0.5 mL of DMF, and filtered through a PTFE frit. The crude residue was purified via preparative LC/MS with the following conditions: Column: XBridge C18, 200 mm×19 mm, 5-μm particles; Mobile Phase A: 5:95 acetonitrile:water with 0.1% TFA; Mobile Phase B: 95:5 acetonitrile:water with 0.1% TFA; Gradient: a 3-minute hold at 0% B, 0-30% B over 28 minutes, then a 0-minute hold at 100% B; Flow Rate: 20 mL/min; Column Temperature: 25° C. Fraction collection was triggered by MS signals. Fractions containing the desired product were combined and dried via centrifugal evaporation to afford Compound 112, as its TFA salt (4.7 mg).

Compound 113, Compound 114, and Compound 116 were analogously prepared.

### Example 11—Compound 126

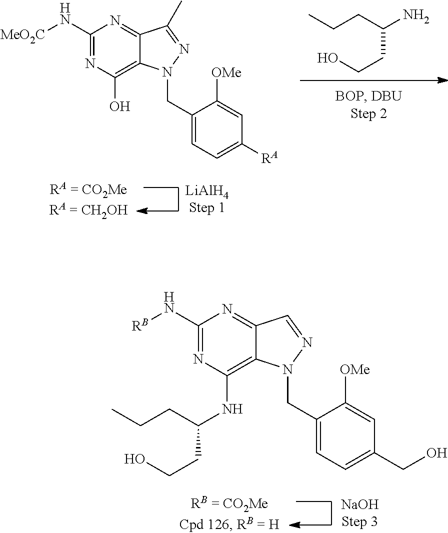

Step 1. A solution of methyl 4-((7-hydroxy-5-((methoxycarbonyl)amino)-3-methyl-1H-pyrazolo[4,3-d]pyrimidin-1-yl)methyl)-3-methoxybenzoate, AcOH (1500 mg, 3.25 mmol) and THF (100 ml) was cooled to 0° C. and treated with LiAlH₄ (1M THF, 6.50 mL, 6.50 mmol), added in 4 portions over 20 min. An additional portion of LiAlH₄ (1M THF, 2 mL, 2.000 mmol) was added. The reaction was quenched with 20% aqueous sodium potassium tartrate solution (100 mL) and stirred for 16 h. Saturated aqueous NaCl solution (100 mL) was added and the reaction was extracted with EtOAc (3×200 mL). The combined organic layers were dried (Na₂SO₄) and concentrated. Column chromatography (80 g SiO₂, 0 to 20% MeOH—CH₂Cl₂, gradient elution) afforded methyl (7-hydroxy-1-(4-(hydroxymethyl)-2-methoxybenzyl)-3-methyl-1H-pyrazolo[4,3-d]pyrimidin-5-yl)carbamate (873 mg).

LC/MS (M+H) 374.1; LC RT=1.16 min (Method C).

Step 2. Methyl (7-hydroxy-1-(4-(hydroxymethyl)-2-methoxybenzyl)-3-methyl-1H-pyrazolo[4,3-d]pyrimidin-5-yl)carbamate (183.0 mg, 0.490 mmol) was dissolved in DMSO (2451 μl) at RT and added to a reaction vial containing (S)-1-((tert-butyldiphenylsilyl)oxy)hexan-3-amine, HCl (250 mg, 0.637 mmol) and BOP (325 mg, 0.735 mmol). DBU (222 μl, 1.470 mmol) was added and the reaction mixture was heated to 50° C. for 16 h. NaOH (10 M, 735 μl, 7.35 mmol) was added to the reaction and the temperature was increased to 70° C. for 16 h. After cooling to RT, the reaction mixture was purified directly by column chromatography (100 g C18 Gold Column, 10 to 90% MeOH—H2O with 0.1% TFA) to afford methyl (S)-(7-((1-hydroxyhexan-3-yl)amino)-1-(4-(hydroxymethyl)-2-methoxybenzyl)-3-methyl-1H-pyrazolo[4,3-d]pyrimidin-5-yl)carbamate (120.0 mg) as a sticky tan solid.

LC/MS (M+H) 473.4; LC RT=0.73 min (Method A).

Step 3. Methyl (S)-(7-((1-hydroxyhexan-3-yl)amino)-1-(4-(hydroxymethyl)-2-methoxybenzyl)-3-methyl-1H-pyrazolo[4,3-d]pyrimidin-5-yl)carbamate (120 mg, 0.254 mmol) was suspended in dioxane (1270 μl) at RT. NaOH (10 M, 127 μl, 1.270 mmol) was added and the reaction was heated to 70° C. for 12 h and cooled to RT. AcOH (73 μl, 1.270 mmol) was added and 20% of the reaction mixture was removed and concentrated. The crude material was purified via preparative LC/MS with the following conditions: Column: XBridge C18, 200 mm×19 mm, 5-μm particles; Mobile Phase A: 5:95 acetonitrile:water with NH₄OAc; Mobile Phase B: 95:5 acetonitrile:water with NH₄OAc; Gradient: a 0-minute hold at 7% B, 7-47% B over 25 minutes, then a 0-minute hold at 100% B; Flow Rate: 20 mL/min; Column Temperature: 25° C. Fraction collection was triggered by MS signals. Fractions containing the desired product were combined and dried via centrifugal evaporation to afford Compound 126 (8.5 mg).

### Example 12—Compound 127

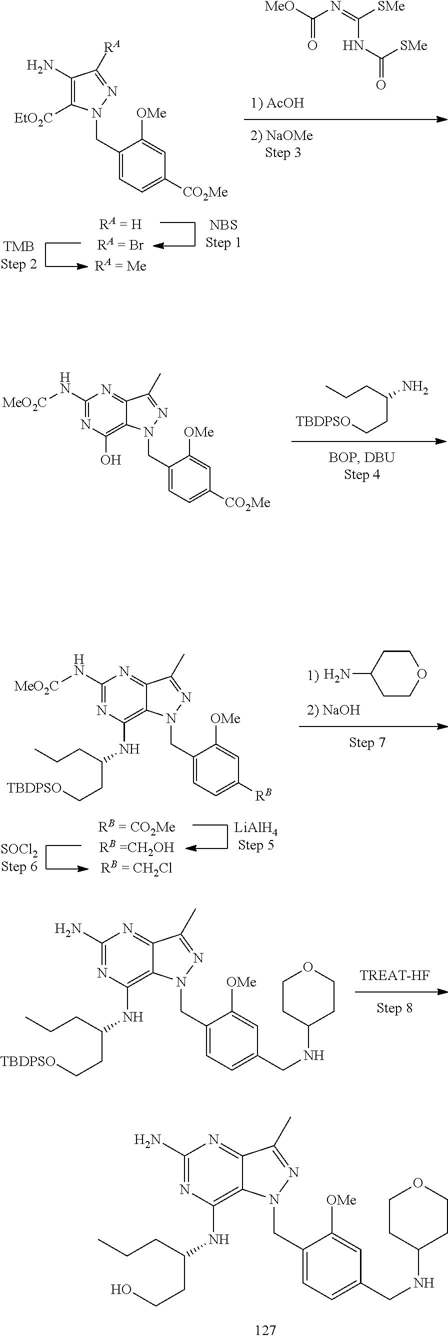

Step 1. Ethyl 4-amino-1-(2-methoxy-4-(methoxycarbonyl)benzyl)-1H-pyrazole-5-carboxylate (1.65 g, 4.95 mmol) was dissolved in CHCl₃ (49.5 ml) and cooled to 0° C. NBS (0.925 g, 5.20 mmol) was added to the reaction mixture in one portion. After 15 minutes, the reaction was diluted with CHCl3 and vigorously stirred with 10% aqueous sodium thiosulfate solution for 10 minutes. The organic phase was separated, washed with H₂O, dried over MgSO₄ and concentrated. The crude product was purified by column chromatography (80 g SiO2, 0 to 50% EtOAc-hexane gradient elution) to afford ethyl 4-amino-3-bromo-1-(2-methoxy-4-(methoxycarbonyl)benzyl)-1H-pyrazole-5-carboxylate (1.32 g) as a white solid.

LC/MS (M+H) 412.2/414.2; LC RT=1.02 min (Method A).

¹H NMR (400 MHz, DMSO-d6) δ 7.61-7.41 (m, 2H), 6.55 (d, J=8.3 Hz, 1H), 5.56 (s, 2H), 5.02 (s, 2H), 4.20 (q, J=7.1 Hz, 2H), 3.90 (s, 3H), 3.85 (s, 3H), 1.15 (t, J=7.1 Hz, 3H).

Step 2. Ethyl 4-amino-1-(2-methoxy-4-(methoxycarbonyl)benzyl)-3-methyl-1H-pyrazole-5-carboxylate (741.2 mg, 67.1% yield), K₂CO₃ (1.098 g, 7.94 mmol) and 2,4,6-trimethyl-1,3,5,2,4,6-trioxatriborinane (TMB, 3.5 M in THF; 1.816 ml, 6.36 mmol) were suspended in dioxane (26.5 ml):water (5.30 ml) (5:1). A stream of N₂ was bubbled through the reaction mixture for 5 min before the addition of PdCl₂(dppf)-CH₂Cl₂ adduct (0.052 g, 0.064 mmol) and continued for another 4 min before sealing the reaction vessel and heating to 90° C. After 3 h, additional portions of TMB (3.5 M in THF; 0.908 ml, 3.18 mmol) and PdCl₂(dppf)-CH₂Cl₂ adduct (0.052 g, 0.064 mmol) were added and the reaction mixture was stirred at 100° C. for 16 hours. The cooled reaction mixture was diluted with 100 mL of EtOAc and filtered through CELITE™, washing with additional EtOAc. The crude product was concentrated onto 4 g CELITE™. Column chromatography (80 g SiO2, 0 to 30% EtOAc-CH₂Cl₂ gradient elution) afforded the expected product, ethyl 4-amino-1-(2-methoxy-4-(methoxycarbonyl)benzyl)-3-methyl-1H-pyrazole-5-carboxylate (741 mg) as a cream colored solid.

LC/MS (M+H) 348.2; LC RT=0.89 min (Method A).

¹H NMR (400 MHz, DMSO-d6) δ 7.49 (d, J=1.5 Hz, 1H), 7.46 (dd, J=7.9, 1.5 Hz, 1H), 6.40 (d, J=7.8 Hz, 1H), 5.48 (s, 2H), 4.94-4.86 (m, 2H), 4.14 (q, J=7.0 Hz, 2H), 3.90 (s, 3H), 3.84 (s, 3H), 2.10 (s, 3H), 1.15-1.08 (m, 3H).

Step 3. Ethyl 4-amino-1-(2-methoxy-4-(methoxycarbonyl)benzyl)-3-methyl-1H-pyrazole-5-carboxylate (742 mg, 2.136 mmol) was suspended in MeOH (10.800 mL) and heated with vigorous stirring to solubilize it. 1,3-bis-(Methoxycarbonyl)-2-methyl-2-thiopseudourea (661 mg, 3.20 mmol), was added followed by AcOH (0.611 mL, 10.68 mmol). The reaction mixture was stirred at RT for 16 h. An additional portion of AcOH was added (0.049 mL, 0.854 mmol) followed by stirring at RT for another 72 h before the addition of NaOMe (25% wt in MeOH, 5.69 mL, 25.6 mmol). After stirring for 3 h, the reaction mixture was re-acidified with AcOH. The product was collected by filtration, air-dried for 10 minutes and thoroughly dried in a chem-dry oven to afford methyl 4-((7-hydroxy-5-((methoxycarbonyl)amino)-3-methyl-1H-pyrazolo[4,3-d]pyrimidin-1-yl)methyl)-3-methoxybenzoate (722.0 mg) as a cream colored solid.

LC/MS (M+H) 402.3; LC RT=0.86 min (Method A).

¹H NMR (400 MHz, DMSO-d6) δ 11.58-11.17 (m, 2H), 7.51 (d, J=1.4 Hz, 1H), 7.49-7.42 (m, 1H), 6.67 (d, J=7.9 Hz, 1H), 5.67 (s, 2H), 3.90 (s, 3H), 3.84 (s, 3H), 3.71 (s, 3H), 2.31 (s, 3H).

Step 4. Methyl 4-((7-hydroxy-5-((methoxycarbonyl)amino)-3-methyl-1H-pyrazolo-[4,3-d]pyrimidin-1-yl)methyl)-3-methoxybenzoate (300 mg, 0.747 mmol), (S)-1-((tert-butyl-diphenylsilyl)oxy)hexan-3-amine, HCl (381 mg, 0.972 mmol) and BOP (496 mg, 1.121 mmol) were suspended in DMF (3737 μL) at RT. After the addition of DBU (4 eq) (451 μl, 2.99 mmol), the reaction mixture became homogenous and was heated to 40° C. After 15 min, an additional portion of DBU (2 eq) (225 μl, 1.495 mmol) was added and the reaction was stirred at 40° C. for 16 h. (S)-1-((tert-butyldiphenylsilyl)oxy)hexan-3-amine, HCl (381 mg, 0.972 mmol), BOP (496 mg, 1.121) and DBU (4 eq) (451 μl, 2.99 mmol) was added and the reaction was stirred for an additional 48 h. The reaction mixture was diluted with EtOAc and washed with H₂O (2×), and 10% LiCl solution (1×). The organic phase was dried over Na₂SO₄ and concentrated. The crude product was purified by column chromatography (24 g SiO₂, 0 to 80% EtOAc-hexane gradient elution) then further purified (12 g SiO₂, 0 to 70% EtOAc-hexane gradient elution to provide methyl (S)-4-((7-((1-((tert-butyldiphenylsilyl)oxy)hexan-3-yl)amino)-5-((methoxycarbonyl)-amino)-3-methyl-1H-pyrazolo[4,3-d]pyrimidin-1-yl)methyl)-3-methoxybenzoate (270.6 mg).

LC/MS (M+H) 739.7; LC RT=1.04 min (Method A).

Step 5. Methyl (S)-4-((7-((1-((tert-butyldiphenylsilyl)oxy)hexan-3-yl)amino)-5-((methoxycarbonyl)amino)-3-methyl-1H-pyrazolo[4,3-d]pyrimidin-1-yl)methyl)-3-methoxybenzoate (270 mg, 0.365 mmol) was dissolved in THF (3654 μl) at RT. LiAlH₄ (731 μl, 0.731 mmol) was added dropwise over 5 minutes and the reaction mixture was stirred for 15 min at RT. The reaction was quenched with MeOH and Rochelle's Salt. EtOAc was added and the reaction mixture was stirred for 3 h, until the layers had cleared. The organic phase was removed and the aqueous layer was extracted with three additional portions of EtOAc. The combined organic phases were washed with brine, dried over Na₂SO₄ and concentrated. Column chromatography (12 g SiO2, 0 to 100% EtOAc-hexane gradient elution, then 0 to 20% MeOH—CH₂Cl₂) provided the expected material, methyl (S)-(7-((1-((tert-butyldiphenylsilyl)oxy)-hexan-3-yl)amino)-1-(4-(hydroxymethyl)-2-methoxybenzyl)-3-methyl-1H-pyrazolo[4,3-d]pyrimidin-5-yl)carbamate (61.7 mg).

LC/MS (M+H) 711.4; LC RT=1.08 min (Method A).

Step 6. Methyl (S)-(7-((1-((tert-butyldiphenylsilyl)oxy)hexan-3-yl)amino)-1-(4-(hydroxymethyl)-2-methoxybenzyl)-3-methyl-1H-pyrazolo[4,3-d]pyrimidin-5-yl)carbamate (60 mg, 0.084 mmol) was dissolved in CH₂Cl₂ (844 μl) at RT. SOCl₂ (30.8 μL, 0.422 mmol) was added and the reaction for hr. Concentration afforded the expected product, methyl (S)-(7-((1-((tert-butyldiphenylsilyl)oxy)hexan-3-yl)amino)-1-(4-(chloromethyl)-2-methoxybenzyl)-3-methyl-1H-pyrazolo[4,3-d]pyrimidin-5-yl)carbamate (56.6 mg).

LC/MS (M+H) 729.3; LC RT=1.18 min (Method A).

Step 7. Methyl (S)-(7-((1-((tert-butyldiphenylsilyl)oxy)hexan-3-yl)amino)-1-(4-(chloromethyl)-2-methoxybenzyl)-3-methyl-1H-pyrazolo[4,3-d]pyrimidin-5-yl)carbamate (54 mg, 0.074 mmol) was dissolved in acetonitrile (740 l) at RT. Tetrahydro-2H-pyran-4-amine (22.47 mg, 0.222 mmol) was added. The reaction mixture was heated at 80° C. for 3 h and allowed to cool overnight to RT. An additional portion of tetrahydro-2H-pyran-4-amine (5 mg, 0.056 mmol) was added, followed by re-heating at 80° C. for 2 h. The reaction mixture was concentrated and the residue re-dissolved in dioxane:MeOH:10M NaOH (6:2:2, 1 mL) and stirred at 80° C. AcOH (130 μL) was added and the reaction was concentrated in vacuo. The residue was dissolved in EtOAc and washed with NaHCO₃ solution (50% aq). The aqueous layers were extracted into EtOAc (3×). The combined organic phases were dried over Na₂SO₄ and re-concentrated to provide (S)-N7-(1-((tert-butyldiphenylsilyl)oxy)hexan-3-yl)-1-(2-methoxy-4-(((tetrahydro-2H-pyran-4-yl)amino)methyl)benzyl)-3-methyl-1H-pyrazolo[4,3-d]pyrimidine-5,7-diamine (25.3 mg).

LC/MS (M+H) 736.4; LC RT=1.03 min (Method A).

Step 8. (S)-N7-(1-((tert-butyldiphenylsilyl)oxy)hexan-3-yl)-1-(2-methoxy-4-(((tetrahydro-2H-pyran-4-yl)amino)methyl)benzyl)-3-methyl-1H-pyrazolo[4,3-d]pyrimidine-5,7-diamine (25.3 mg, 0.034 mmol) was dissolved in DMF at RT. Triethylamine trihydrofluoride (TREAT-HF, 16.79 μL, 0.103 mmol) was added, followed by stirring at RT for 5 h. The reaction was quenched with NaOH (1N), diluted through at PTFE frit and purified via preparative LC/MS with the following conditions: Column: XBridge C18, 200 mm×19 mm, 5-μm particles; Mobile Phase A: 5:95 acetonitrile:water with NH₄OAc; Mobile Phase B: 95:5 acetonitrile:water with NH₄OAc; Gradient: a 0-minute hold at 5% B, 5-45% B over 20 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation to provide Compound 127 (8.2 mg) as a mono acetate.

The following compounds were analogously prepared: Compound 128, Compound 129, and Compound 130.

### Example 13—Compound 125

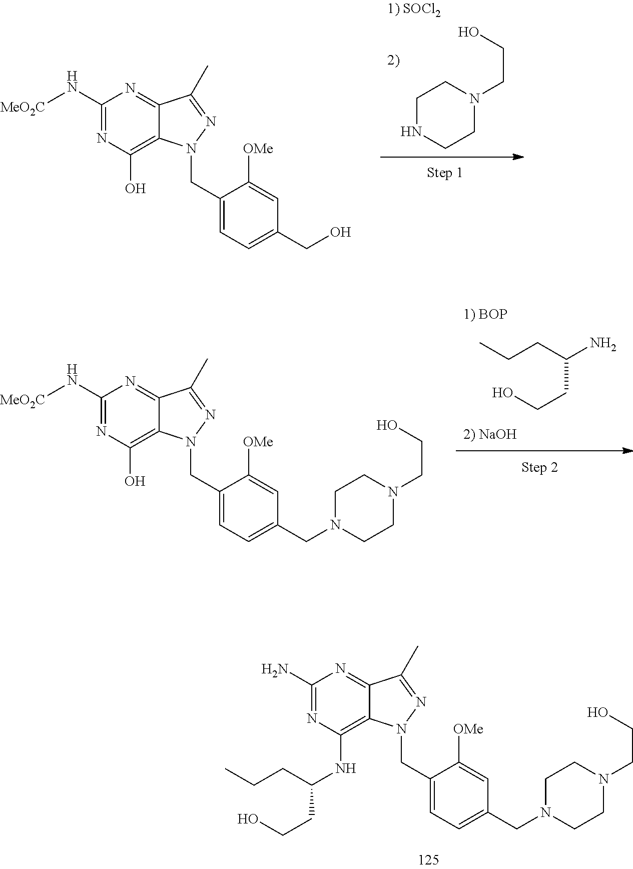

Step 1. To a solution of methyl (7-hydroxy-1-(4-(hydroxymethyl)-2-methoxybenzyl)-3-methyl-1H-pyrazolo[4,3-d]pyrimidin-5-yl)carbamate (426 mg, 1.141 mmol) in THF (10 mL) is added SOCl₂ (0.167 mL, 2.282 mmol). After 30 min the reaction mixture was concentrated and dried under high vacuum. The crude residue was then diluted with DMF (2 mL) and 2-(pipera-zin-1-yl)ethan-1-ol (743 mg, 5.70 mmol) was added. After heating to 80° C. for 30 minutes, the reaction mixture was cooled to RT and partitioned between ethyl acetate (50 mL) and aqueous LiCl 10% (25 mL). The phases were separated and the aqueous phase was extracted with ethyl acetate (2×50 mL). The combined organic layers were dried with Na₂SO₄, filtered and concentrated. The residue was purified by chromatography (SiO₂, MeOH:DCM=0-30%) to give methyl (7-hydroxy-1-(4-((4-(2-hydroxyethyl)piperazin-1-yl)methyl)-2-methoxybenzyl)-3-methyl-1H-pyrazolo[4,3-d]pyrimidin-5-yl)carbamate (255.0 mg, 0.52 mmol, 46% yield).

Step 2. A solution of methyl (7-hydroxy-1-(4-((4-(2-hydroxyethyl)piperazin-1-yl)methyl)-2-methoxybenzyl)-3-methyl-1H-pyrazolo[4,3-d]pyrimidin-5-yl)carbamate (25 mg, 0.051 mmol), (S)-2-aminopentan-1-ol (21.25 mg, 0.206 mmol), BOP (34.2 mg, 0.077 mmol), and DBU (0.016 ml, 0.103 mmol) in dioxane (2 mL) was stirred at RT for 16 h. NaOH 10M (0.1 ml, 1.000 mmol) was added and heated to 80° C. Acetic acid (0.1 ml) is added after an hour and the solvent evaporated under reduced pressure. The residue was purified via preparative LC/MS with the following conditions: Column: XBridge C18, 200 mm×19 mm, 5-μm particles; Mobile Phase A: 5:95 acetonitrile:water with NH₄OAc; Mobile Phase B: 95:5 acetonitrile:water with NH₄OAc; Gradient: a 0-minute hold at 3% B, 3-43% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The yield of Compound 125 was 13.9 mg, and its estimated purity by LCMS analysis was 100%. Analytical LC/MS was used to determine the final purity.

### Example 14—Compound 117

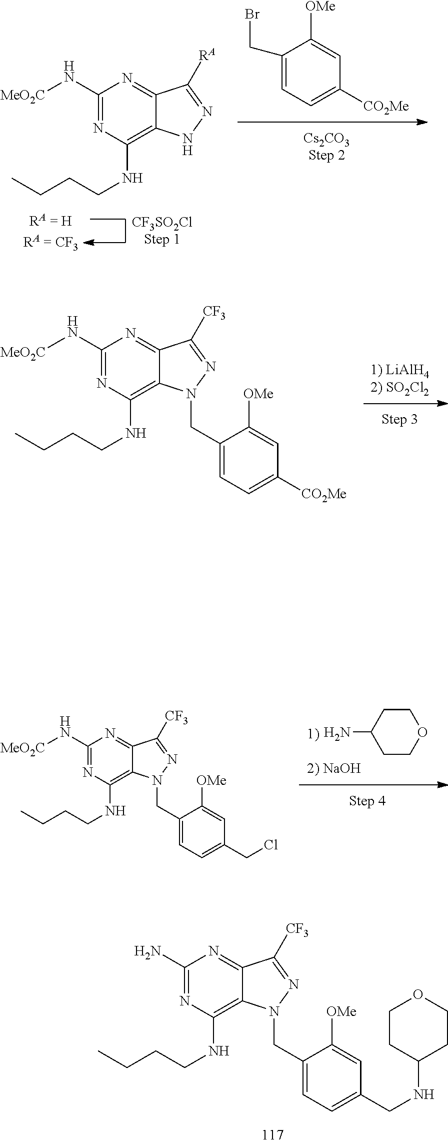

Step 1: In a 8 mL vial equipped with a magnetic stir bar, methyl (7-(butylamino)-1H-pyrazolo[4,3-d]pyrimidin-5-yl)carbamate (0.5 g, 1.892 mmol) and potassium phosphate dibasic (0.983 g, 5.64 mmol) were suspended in dry acetonitrile (5 mL) and stirred well. The mixture was degassed by bubbling N₂ gas through the reaction. Tris(4,7-diphenyl-1,10-phenanthroline)-ruthenium(II) dichloride complex (0.11 g, 0.094 mmol) was added and the reaction was purged with N₂ for an additional 10 min. Trifluoromethanesulfonyl chloride (0.199 ml, 1.882 mmol) was added and then, the reaction was tightly capped and sealed with Parafilm. The reaction mixture was irradiated with stirring for 1 week using a 27 W blue LED lamp. The reaction was quenched with water and extracted with CH₂Cl₂ (3×10 mL). The organic layer was washed with water and brine, dried over anhydrous Na₂SO₄ and concentrated. The crude product was purified via mass-based preparative HPLC with the following conditions: Column: Sunfire C18, 150 mm×19 mm, 5-μm particles; Mobile Phase A: 10 mM NH₄OAc in water; Mobile Phase B: 95:5 acetonitrile:MeOH (1:1); Gradient: a 0-minute hold at 10% B, 10 to 40% B over 2 minutes, then 40 to 75% B over 28 minutes; Flow Rate: 19 mL/min; Column Temperature: 25° C. Fraction collection was triggered by MS signals and fractions containing the product were concentrated to afford methyl (7-(butylamino)-3-(trifluoromethyl)-1H-pyrazolo[4,3-d]pyrimidin-5-yl)carbamate as an off-white solid (130 mg).

¹H NMR (400 MHz, DMSO-d6) δ 13.4 (br s, 1H), 9.86 (s, 1H), 7.70 (s, 1H), 3.65 (s, 3H), 3.56 (m, 2H), 1.63 (m, 2H), 1.38 (m, 2H), 0.96 (t, J=7.2 Hz, 3H).

LC/MS [M+H]⁺ 333.2; LC RT=1.915 min (Column: Kinetex XB-C18 3×75 mm; 2.6μ; Mobile Phase A: 10 mM NH₄OAc in H₂O:acetonitrile (98:2); Mobile Phase B: 10 mM NH₄OAc in H₂O:acetonitrile (2:98); Gradient: 20-100% B over 4.0 min, then a 0.60 min hold at 100% B; Flow: 1.0 mL/min. Detection: MS and UV (220 nm).

Step 2: Methyl (7-(butylamino)-3-(trifluoromethyl)-1H-pyrazolo[4,3-d]pyrimidin-5-yl)carbamate (120 mg, 0.361 mmol) and Cs₂CO₃ (129 mg, 0.397 mmol) were suspended in DMF (1806 μl) at RT and treated with methyl 4-(bromomethyl)-3-methoxybenzoate (89 mg, 0.343 mmol). The reaction mixture was stirred at RT overnight and partitioned between EtOAc and H₂O. The organic layer was separated and the aqueous layer was extracted with EtOAc (2×). The combined organic phases were washed with 10% LiCl solution and brine and dried ver Na₂SO₄. Column chromatography (12 g SiO₂, 10 to 100% EtOAc-hexane gradient elution) afforded the crude product as a mixture of N1 and N2 isomers. The isomers were separated via reverse phase purification (15.5 g C18 Gold, 10 to 90% MeOH—H₂O with 0.05% TFA, gradient elution). The fractions containing the product peak were partially concentrated to remove the MeOH. The residual aqueous solution was treated with saturated NaHCO₃ solution and extracted with CH₂Cl₂ (3×). The combined organic phases were dried over Na₂SO₄, concentrated and dried under vacuum to afford methyl 4-((7-(butylamino)-5-((methoxycarbonyl)amino)-3-(trifluoromethyl)-1H-pyrazolo[4,3-d]pyrimidin-1-yl)methyl)-3-methoxybenzoate (52.4 mg).

¹H NMR (400 MHz, DMSO-d6) δ 9.92 (s, 1H), 7.54-7.48 (m, 2H), 7.40 (br t, J=5.4 Hz, 1H), 6.85 (d, J=8.3 Hz, 1H), 5.87 (s, 2H), 3.84 (d, J=1.1 Hz, 6H), 3.62 (s, 3H), 3.52 (q, J=6.6 Hz, 2H), 1.55 (quin, J=7.2 Hz, 2H), 1.20 (dq, J=14.9, 7.4 Hz, 2H), 0.83 (t, J=7.4 Hz, 3H)

Step 3: Methyl 4-((7-(butylamino)-5-((methoxycarbonyl)amino)-3-(trifluoro-methyl)-1H-pyrazolo[4,3-d]pyrimidin-1-yl)methyl)-3-methoxybenzoate (52 mg, 0.102 mmol) was suspended in THF at RT and stirred until dissolved. LiAlH₄ (204 μL, 0.204 mmol) was added slowly dropwise. The reaction was sonicated and stirred at RT for 10 min before quenching with 2 drops of MeOH. The reaction mixture was diluted with EtOAc and Rochelle's salt and stirred at RT for 90 min. The organic phase was separated and the aqueous phase was extracted with EtOAc (3×). The combined organic phases were dried over Na₂SO₄ and concentrated. The crude methyl (7-(butylamino)-1-(4-(hydroxymethyl)-2-methoxybenzyl)-3-(trifluoromethyl)-1H-pyrazolo[4,3-d]pyrimidin-5-yl)carbamate was further dried in vacuum (38 mg). The crude material was dissolved in THF and treated with SOCl₂ (37 μL, 0.05 mmol). The reaction mixture was stirred for 2.5 h, concentrated and azeotroped with acetonitrile (1×) to afford methyl (7-(butylamino)-1-(4-(chloromethyl)-2-methoxybenzyl)-3-(trifluoromethyl)-1H-pyrazolo[4,3-d]pyrimidin-5-yl)carbamate (41 mg).

LC/MS [M+H]⁺ 501.0. LC RT=0.89 min. (Method A).

Step 4: Methyl (7-(butylamino)-1-(4-(chloromethyl)-2-methoxybenzyl)-3-(trifluoromethyl)-1H-pyrazolo[4,3-d]pyrimidin-5-yl)carbamate (41 mg, 0.082 mmol) was dissolved in DMF (409 μl) at RT and treated with tetrahydro-2H-pyran-4-amine (33.9 μl, 0.327 mmol). The reaction was stirred at RT overnight. 10 M aqueous NaOH solution (41 μL) was added and the reaction was heated to 80° C. for 1 h. The cooled reaction mixture containing the crude product was treated with AcOH (23 μL), diluted with DMF (1 mL) and filtered through a PTFE frit. The crude material was purified via preparative LC/MS with the following conditions: Column: XBridge C18, 200 mm×19 mm, 5-μm particles; Mobile Phase A: 5:95 acetonitrile:water with 0.05% TFA; Mobile Phase B: 95:5 acetonitrile:water with 0.05% TFA; Gradient: a 0-minute hold at 6% B, 6-56% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile:water with NH₄OAc; Mobile Phase B: 95:5 acetonitrile:water with NH₄OAc; Gradient: a 0-minute hold at 18% B, 18-58% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation to afford Compound 117 (1.4 mg).

### Example 15—Compound 131

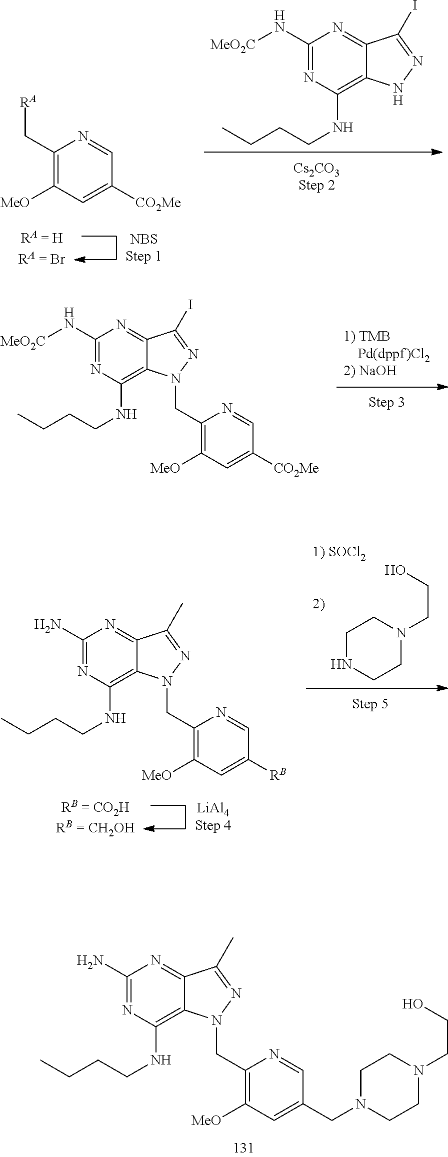

Step 1. A suspension of methyl 5-methoxy-6-methylnicotinate (993.5 mg, 5.48 mmol, commercially available from Enamine), NBS (1074.0 mg, 6.03 mmol) and AIBN (180.0 mg, 1.10 mmol) in 20 mL CCl₄ was stirred at 65° C. for 6 h. The solution was then cooled and evaporated under reduced pressure. The final residue was purified by chromotography (SiO₂, EtOAc:Hexane=0-20%) to give methyl 6-(bromomethyl)-5-methoxynicotinate (917.0 mg, 3.53 mmol, 64.3% yield) as a pink solid.

LC/MS at t=1.614 min (Method 3), MS (ESI) calcd. [M+H]⁺ 260.0, found 260.2.

Step 2. To a 100 mL flask was charged with methyl (7-(butylamino)-3-iodo-1H-pyrazolo[4,3-d]pyrimidin-5-yl)carbamate (see Example 1 above; 1447 mg, 3.71 mmol), Cs₂CO₃ (2416 mg, 7.41 mmol) and DMF (24 mL) to form a suspension at RT. Methyl 6-(bromomethyl)-5-methoxynicotinate (916.0 mg, 3.52 mmol) was added as a DMF (6 mL) solution. After 20 min, 100 mL 20% ammonium chloride and 200 mL EtOAc were added. During workup, white solid precipitated out and collected by filtration to give methyl 6-((7-(butylamino)-3-iodo-5-((methoxycarbonyl)amino)-1H-pyrazolo[4,3-d]pyrimidin-1-yl)methyl)-5-methoxynicotinate (1.70 g, 2.99 mmol, 81% yield) as a white solid.

LC/MS at t=1.738 min (Method 3), MS (ESI) calcd. [M+H]⁺ 570.1, found 570.0.

¹H NMR (400 MHz, DMSO-d6) δ 9.77 (s, 1H), 8.47 (d, J=1.7 Hz, 1H), 7.84 (d, J=1.7 Hz, 1H), 7.55 (t, J=5.6 Hz, 1H), 5.95 (s, 2H), 3.98 (s, 3H), 3.87 (s, 3H), 3.63 (s, 3H), 3.48 (q, J=6.6 Hz, 2H), 1.53 (m, 2H), 1.27-1.13 (m, 2H), 0.83 (t, J=7.4 Hz, 3H).

Step 3. To a 10 mL microwave vial was charged with methyl 6-((7-(butylamino)-3-iodo-5-((methoxycarbonyl)amino)-1H-pyrazolo[4,3-d]pyrimidin-1-yl)methyl)-5-methoxynicotinate (320.7 mg, 0.563 mmol), K₂CO₃ (389.0 mg, 2.82 mmol), dioxane (3 mL) and H2O (0.3 mL) at RT. Nitrogen was bubbled through the suspension for 10 min before [1,1′-bis(diphenyl-phosphino)ferrocene]dichloropalladium(II) (41.2 mg, 0.056 mmol) was added. The mixture was microwaved at 120° C. for 1 h and taken up by 8 mL MeOH and 2 mL NaOH (10 M). The mixture was then stirred at 70° C. for 1 h before it was cooled down and filtered. The precipitate was washed with more MeOH and the filtrated was evaporated. The final residue was purified by chromatography (C₁₈, acetonitrile:Water=0-100%, with 10 mM NH₄OAc in both eluents) to give 6-((5-amino-7-(butylamino)-3-methyl-1H-pyrazolo[4,3-d]pyrimidin-1-yl)methyl)-5-methoxynicotinic acid (130 mg, 0.338 mmol, 60% yield).

LC/MS at t=1.683 min (Method 3), MS (ESI) calcd. [M+H]⁺ 458.2, found 458.2.

Step 4. To a 100 mL flask was charged with 6-((5-amino-7-(butylamino)-3-methyl-1H-pyrazolo[4,3-d]pyrimidin-1-yl)methyl)-5-methoxynicotinic acid (96.0 mg, 0.249 mmol) and THF (5 mL) to form a suspension. LiAlH₄ (2M in THF) (0.374 mL, 0.747 mmol) was added at 5° C. (ice bath). After 1 h, more LiAlH₄ (0.374 mL, 0.747 mmol) was added. After another 1 h, 3 mL 20% Rochelle's salt was added. 100 mL MeOH was also added. The mixture was stirred at RT over the weeked. MeOH was evaporated off and 3 mL DMSO was added. The mixture was purified by chromatography (C18, acetonitrile:water=0-100%, with 0.1% TFA in both eluents) to give 40.0 mg product (about 50% purity), which was used for next step without further purification.

LC/MS at t=1.321 min (Method 3), MS (ESI) calcd. [M+H]⁺ 372.2, found 372.1.

Step 5. To a 4 mL vial was charged with (6-((5-amino-7-(butylamino)-3-methyl-1H-pyrazolo[4,3-d]pyrimidin-1-yl)methyl)-5-methoxypyridin-3-yl)methanol (40 mg, 0.05 mmol, about 50% purity) and Dioxane (1 mL) to form a suspension (a little bit of solubility issue at this concentration). SOCl₂ (0.039 mL, 0.538 mmol) was added at 5° C. (ice bath). The temperature was raised to RT in 5 min. After 30 min, the mixture was evaporaterd and the residue was dissolved in 1 mL DMF. 2-(piperazin-1-yl)ethan-1-ol (0.050 mL, 0.390 mmol) and Hunig's base (0.030 mL, 0.172 mmol) were added. The reaction mixture was stirred at RT for 1 h before it was evaporated down and the residue was purified by chromatography (Column: XBridge C18, 200 mm×19 mm, 5-μm particles; Mobile Phase A: 5:95 acetonitrile:water with NH₄OAc; Mobile Phase B: 95:5 acetonitrile:water with NH₄OAc; Gradient: a 0-minute hold at 5% B, 5-45% B over 20 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation to give Compound 131 (9.4 mg, 0.019 mmol, 36% yield).

### Example 16—Compound 132

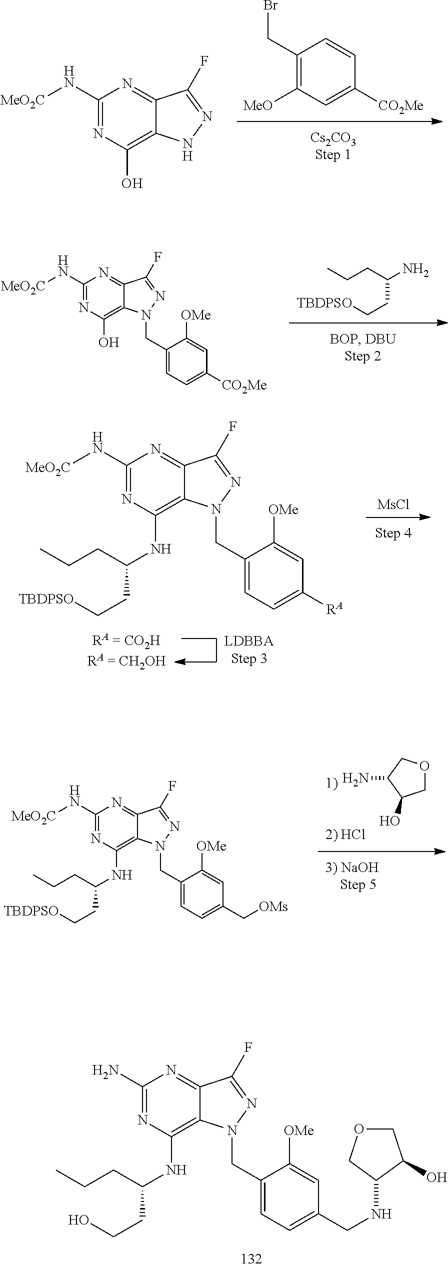

Step 1. To methyl (3-fluoro-7-hydroxy-1H-pyrazolo[4,3-d]pyrimidin-5-yl)carbamate (1 g, 4.40 mmol) in DMF (15 mL) was added Cs₂CO₃ (4.30 g, 13.21 mmol) and the mixture stirred at 0° C. (ice bath) for 10 min. Methyl 4-(bromomethyl)-3-methoxybenzoate (1.2 g, 4.63 mmol) was added and the ice bath was removed after 30 min. The reaction mixture was stirred at 25° C. 12 h. The reaction mixture was diluted with 300 mL water; extracted with EtOAc, and dried over Na₂SO₄. The solvent was removed to afford methyl 4-((3-fluoro-7-hydroxy-5-((methoxycarbonyl)amino)-1H-pyrazolo[4,3-d]pyrimidin-1-yl)methyl)-3-methoxybenzoate (1.78 g, 4.39 mmol, 100% yield). The material was used without purification.

Step 2. A mixture of methyl 4-((3-fluoro-7-hydroxy-5-((methoxycarbonyl)amino)-1H-pyrazolo[4,3-d]pyrimidin-1-yl)methyl)-3-methoxybenzoate (0.93 g, 2.294 mmol) in DMSO (10 mL) was treated with (S)-1-((tert-butyldiphenylsilyl)oxy)hexan-3-amine (2.5 g, 7.03 mmol), 2,3,4,6,7,8,9,10-octahydropyrimido[1,2-a]azepine (DBU, 1.5 ml, 9.95 mmol) followed by ((1H-benzo[d][1,2,3]triazol-1-yl)oxy)tris(dimethylamino)phosphonium hexafluorophosphate(V) (BOP, 2.030 g, 4.59 mmol) and heated at 70° C. for 2 h. The reaction was diluted with EtOAc and washed with water. The solvent mixture was dried over Na₂SO₄ and solvent removed. The material was purified on silica gel (dry load) hexane-EtOAc 0-100% to afford methyl (S)-4-((7-((1-((tert-butyldiphenylsilyl)oxy)hexan-3-yl)amino)-3-fluoro-5-((methoxycarbonyl)amino)-1H-pyrazolo[4,3-d]pyrimidin-1-yl)methyl)-3-methoxybenzoate (1.7 g, 2.288 mmol, 100% yield).

Step 3. To a solution of methyl (S)-4-((7-((1-((tert-butyldiphenylsilyl)oxy)hexan-3-yl)amino)-3-fluoro-5-((methoxycarbonyl)amino)-1H-pyrazolo[4,3-d]pyrimidin-1-yl)methyl)-3-methoxybenzoate (1.7 g, 2.288 mmol) in THF (20 ml) was added lithium diisobutyl-tert-butoxyaluminum hydride solution (LDBBA), 0.25 M in THF/hexanes (24 ml, 6.00 mmol) at 0° C. in 5 mins and the reaction was allowed to keep stirring at RT 12 h (3 h, 98% conversion). LCMS showed 100% conversion. The solution was diluted with cold water and extracted with AcOEt 3 times. Finally, the organic layer was dried over Na2SO4 and evaporated under vacuum. The material was purified on silica gel (hexane-EtOAc 0-100%) to afford methyl (S)-(7-((1-((tert-butyldiphenylsilyl)oxy)hexan-3-yl)amino)-3-fluoro-1-(4-(hydroxymethyl)-2-methoxybenzyl)-1H-pyrazolo[4,3-d]pyrimidin-5-yl)carbamate (300 mg, 0.420 mmol, 18.34% yield).

Step 4. In a 20 dram vial, methyl (S)-(7-((1-((tert-butyldiphenylsilyl)oxy)hexan-3-yl)amino)-3-fluoro-1-(4-(hydroxymethyl)-2-methoxybenzyl)-1H-pyrazolo[4,3-d]pyrimidin-5-yl)carbamate (280 mg, 0.392 mmol) was dissolved in anhydrous CH2Cl2 (4 ml) to give a clear solution at RT. The solution was cooled to 0° C.; MsCl (0.164 ml, 1.175 mmol) and Et₃N (0.164 ml, 1.175 mmol) was added. LCMS at 3 min showed the completion of the reaction. The reaction was quenched with ice water and DCM. The DCM layer was washed with brine (milky aqueous layer became clear when brine was used), dried over Na₂SO₄ and concentrated to give (S)-4-((7-((1-((tert-butyldiphenylsilyl)oxy)hexan-3-yl)amino)-3-fluoro-5-((methoxycarbonyl)-amino)-1H-pyrazolo[4,3-d]pyrimidin-1-yl)methyl)-3-methoxybenzyl methanesulfonate (60 mg, 19.32% yield). The material was used without purification.

Step 5. To (S)-4-((7-((1-((tert-butyldiphenylsilyl)oxy)hexan-3-yl)amino)-3-fluoro-5-((methoxycarbonyl)amino)-1H-pyrazolo[4,3-d]pyrimidin-1-yl)methyl)-3-methoxybenzyl methanesulfonate (60 mg, 0.076 mmol) in DMF (1 mL) was added (3S,4R)-4-aminotetrahydro-furan-3-ol (25 mg, 0.242 mmol) and the reaction was stirred 12 h at 25° C. LC/MS confirmed the alkylation and HCl in 1,4-dioxane (3 mL, 12.00 mmol) was added; the mixture was stirred 2 h at 25° C. The solvent was removed and the LC/MS confirmed the removal of the silyl protecting group. The material was diluted with NaOH (2 mL, 10.00 mmol) in MeOH and stirred at 80° C. for 1 hr. The LC/MS confirmed the desired material and the solvent was removed. The material was diluted with DMSO 4 ml and filtered. The crude product was purified via preparative LC/MS with the following conditions: Column: XBridge C18, 200 mm×19 mm, 5-μm particles; Mobile Phase A: 5:95 acetonitrile:water with 0.1% TFA; Mobile Phase B: 95:5 acetonitrile:water with 0.1% TFA; Gradient: a 0-minute hold at 0% B, 0-40% B over 20 minutes, then a 0-minute hold at 30 100% B; Flow Rate: 20 mL/min; Column Temperature: 25° C. Fraction collection was triggered by MS and UV signals. Fractions containing the desired product were combined and dried via centrifugal evaporation to afford Compound 132 (13.9 mg, 0.028 mmol, 36.5% yield).

Compound 133 was analogously prepared.

### Example 17—Compound 134

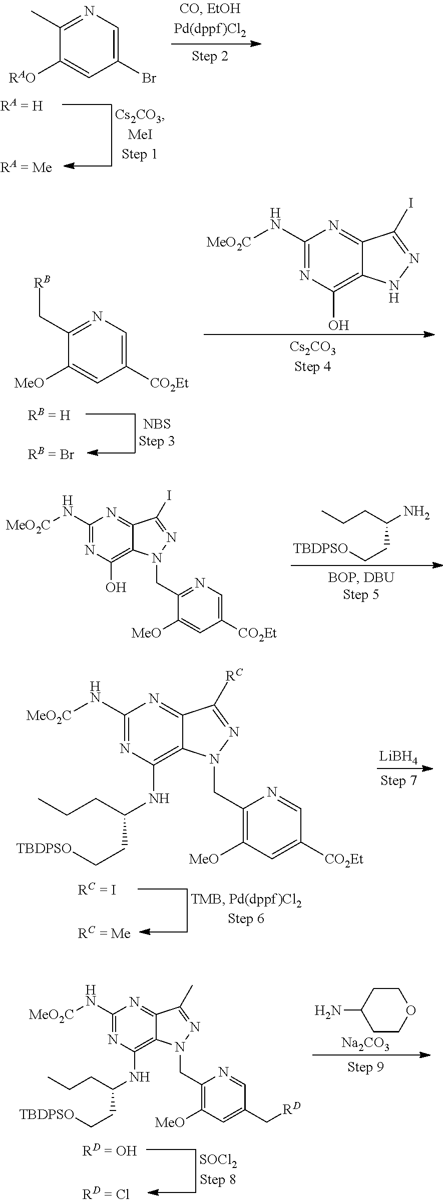

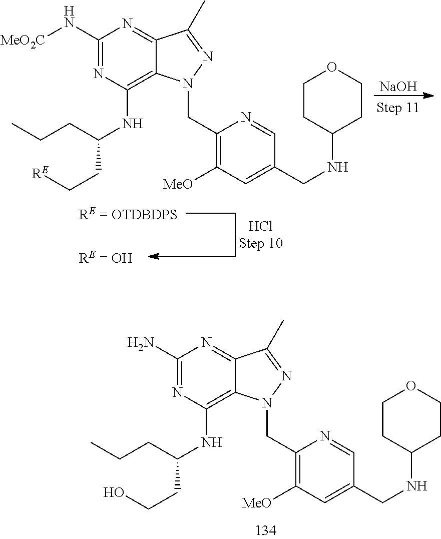

Step 1. To a stirred solution of 5-bromo-2-methylpyridin-3-ol (5.0 g, 26.6 mmol) in acetonitrile (25.0 mL), were added Cs₂CO₃ (17.33 g, 53.2 mmol) and Mel (3.33 mL, 53.2 mmol). The reaction mixture was stirred at RT for 90 min and then partitioned between EtOAc and water. The organic layer was washed with brine, dried over Na₂SO₄, filtered and concentrated under reduced pressure to afford an oily residue, which was dissolved in petroleum ether and filtered. The filtrate was concentrated under reduced pressure to afford 5-bromo-3-methoxy-2-methylpyridine (3.8 g, 16.36 mmol, 61.5% yield) as a brown oil.

¹H NMR (300 MHz, DMSO-d₆) δ=8.16-8.06 (m, 1H), 7.64-7.53 (m, 1H), 3.85 (s, 3H), 2.31 (s, 3H).

LC-MS m/z 202.0 [M+H]⁺.

Step 2. To a stirred solution of 5-bromo-3-methoxy-2-methylpyridine (3.5 g, 17.32 mmol) in a solvent mixture of DMF (50.0 mL) and ethanol (50.0 mL), were added TEA (7.24 mL, 52.0 mmol), PdCl₂(dppf).CH₂Cl₂ adduct (2.83 g, 3.46 mmol) with nitrogen purging. The reaction mixture was stirred at 100° C. under CO gas with 10 bar pressure in an autoclave for 16 h. The reaction mixture was concentrated under reduced pressure to afford a residue, which was taken in DCM and filtered through a CELITE™ bed that was subsequently washed with an excess of DCM. Then filtrate was concentrated under reduced pressure to afford a residue. The crude product was purified by ISCO Combiflash chromatography by eluting with 0-100% ethyl acetate in petroleum ether to afford ethyl 5-methoxy-6-methylnicotinate (2.92 g, 13.46 mmol, 78% yield) as a brown oil.

¹H NMR (300 MHz, DMSO-d₆) δ=8.58-8.53 (m, 1H), 7.68-7.63 (m, 1H), 4.35 (q, J=7.2 Hz, 2H), 3.90 (s, 3H), 2.45-2.40 (m, 3H), 1.38-1.29 (m, 3H).

LC-MS m/z 196.0 [M+H]⁺.

Step 3. To a stirred solution of ethyl 5-methoxy-6-methylnicotinate (6.5 g, 33.3 mmol) in CCl₄ (65.0 mL), were added NBS (7.11 g, 40.0 mmol) and AIBN (1.094 g, 6.66 mmol). The reaction mixture was stirred at 65° C. for 18 h. The reaction mixture was filtered through a CELITE™ bed, which was then washed with excess DCM. The filtrate was concentrated under reduced pressure to afford the residue. The crude product was purified by ISCO Combiflash chromatography by eluting with 0-100% ethyl acetate in petroleum ether to afford ethyl 6-(bromomethyl)-5-methoxynicotinate (3.72 g, 13.03 mmol, 39.1% yield) as a light brown solid.

¹H NMR (300 MHz, DMSO-d₆) δ=8.64 (s, 1H), 7.83 (d, J=1.9 Hz, 1H), 4.69-4.65 (m, 2H), 4.42-4.32 (m, 2H), 4.00-3.95 (m, 3H), 1.39-1.30 (m, 3H).

LC-MS m/z 276.0 [M+H]⁺.

Step 4. To a stirred solution of methyl (7-hydroxy-3-iodo-1H-pyrazolo[4,3-d]pyrimidin-5-yl)carbamate (5.5 g, 16.41 mmol) in DMF (20.0 mL), was added Cs₂CO₃ (10.70 g, 32.8 mmol). The mixture was cooled to 0° C. and ethyl 6-(bromomethyl)-5-methoxynicotinate (4.50 g, 16.41 mmol) was added. The reaction mixture was stirred at 0° C. for 1 h. Water was added. The precipitated solid was filtered and washed with excess of water followed by petroleum ether. The solid was dried under vacuum to afford ethyl 6-((7-hydroxy-3-iodo-5-((methoxycarbonyl)amino)-1H-pyrazolo[4,3-d]pyrimidin-1-yl)methyl)-5-methoxynicotinate (4.1 g, 6.60 mmol, 40.2% yield) as a light brown solid.

¹H NMR (300 MHz, DMSO-d₆) δ=11.68-11.35 (m, 1H), 8.51-8.46 (m, 1H), 7.98-7.93 (m, 1H), 7.80 (d, J=1.5 Hz, 1H), 5.90-5.84 (m, 2H), 4.40-4.29 (m, 2H), 3.96 (s, 3H), 3.74-3.65 (m, 3H), 2.89 (s, 3H), 2.73 (s, 3H), 1.67 (s, 3H), 1.31 (t, J=7.2 Hz, 3H).

LC-MS m/z 529.0 [M+H]⁺.

Step 5. To a stirred solution of ethyl 6-((7-hydroxy-3-iodo-5-((methoxycarbonyl)-amino)-1H-pyrazolo[4,3-d]pyrimidin-1-yl)methyl)-5-methoxynicotinate (3.9 g, 7.38 mmol) in DMSO (30.0 mL), were added DBU (3.34 mL, 22.15 mmol), BOP (4.90 g, 11.07 mmol) and (S)-1-((tert-butyldiphenylsilyl)oxy)hexan-3-amine (2.63 g, 7.38 mmol). The reaction mixture was stirred at 45° C. for 2 h. The mixture was partitioned between EtOAc and water. The organic layer was washed with brine, dried over Na₂SO₄, filtered and concentrated under reduced pressure to afford a residue. The crude compound was purified by ISCO Combiflash chromatography by eluting with 0-100% ethyl acetate in petroleum ether to afford ethyl (S)-6-((7-((1-((tert-butyldiphenylsilyl)oxy)hexan-3-yl)amino)-3-iodo-5-((methoxycarbonyl)amino)-1H-pyrazolo[4,3-d]pyrimidin-1-yl)methyl)-5-methoxynicotinate (1.98 g, 1.921 mmol, 26.0% yield) as a light brown solid.

¹H NMR (300 MHz, DMSO-d₆) δ=9.75 (s, 1H), 8.31-8.27 (m, 1H), 7.82 (d, J=1.5 Hz, 1H), 7.63-7.30 (m, 11H), 7.29-7.13 (m, 4H), 5.87 (s, 2H), 4.69-4.56 (m, 1H), 4.28 (q, J=7.2 Hz, 2H), 3.97 (s, 3H), 3.70-3.61 (m, 2H), 3.58 (s, 3H), 1.92-1.78 (m, 2H), 1.52 (q, J=7.8 Hz, 2H), 1.26-1.25 (m, 1H), 1.28-1.22 (m, 4H), 1.19 (br d, J=7.2 Hz, 2H), 1.02-0.95 (m, 1H), 0.91 (s, 9H), 0.79 (t, J=7.2 Hz, 3H).

LC-MS m/z 866.1 [M+H]⁺.

Step 6. To a stirred solution of ethyl (S)-6-((7-((1-((tert-butyldiphenylsilyl)oxy)hexan-3-yl)amino)-3-iodo-5-((methoxycarbonyl)amino)-1H-pyrazolo[4,3-d]pyrimidin-1-yl)methyl)-5-methoxynicotinate (1.8 g, 2.079 mmol) in 1,4-dioxane (15.0 mL), were added K₂CO₃ (0.575 g, 4.16 mmol), trimethylboroxine (TMB, 0.522 g, 4.16 mmol) and PdCl₂(dppf).CH₂Cl₂ adduct (0.170 g, 0.208 mmol) under nitrogen purging. The reaction mixture was stirred at 100° C. for 6 h. The mixture was filtered through CELITE™ bed, which was then washed with excess EtOAc. The filtrate was concentrated under reduced pressure to afford a residue, which was purified by ISCO Combiflash chromatography by eluting with 50-90% ethyl acetate in petroleum ether to afford ethyl (S)-6-((7-((1-((tert-butyldiphenylsilyl)oxy)hexan-3-yl)amino)-5-((methoxycarbonyl)amino)-3-methyl-1H-pyrazolo[4,3-d]pyrimidin-1-yl)methyl)-5-methoxynicotinate (0.52 g, 0.600 mmol, 28.9% yield) as a red solid.

¹H NMR (300 MHz, DMSO-d₆) δ=9.54 (s, 1H), 8.35-8.29 (m, 1H), 7.84-7.77 (m, 1H), 7.55 (dd, J=1.5, 7.9 Hz, 2H), 7.50-7.45 (m, 2H), 7.40-7.28 (m, 5H), 7.24-7.18 (m, 2H), 7.12 (br d, J=8.3 Hz, 1H), 5.75 (s, 2H), 4.60 (br d, J=7.2 Hz, 1H), 4.29 (q, J=7.1 Hz, 2H), 3.96 (s, 3H), 3.72-3.63 (m, 2H), 3.57 (s, 3H), 2.29 (s, 3H), 1.91-1.77 (m, 2H), 1.61-1.46 (m, 2H), 1.25 (t, J=7.2 Hz, 5H), 0.91 (s, 10H), 0.84-0.76 (m, 3H).

LC-MS m/z 754.3 [M+H]⁺.

Step 7. To a stirred solution of ethyl (S)-6-((7-((1-((tert-butyldiphenylsilyl)oxy)hexan-3-yl)amino)-5-((methoxycarbonyl)amino)-3-methyl-1H-pyrazolo[4,3-d]pyrimidin-1-yl)methyl)-5-methoxynicotinate (0.4 g, 0.531 mmol) in THF (7.0 mL) and MeOH (3.0 mL), was added LiBH₄ (2.0 M in THF) (2.65 mL, 5.31 mmol). The reaction mixture was stirred at 45° C. for 16 h. The reaction mixture was quenched with saturated ammonium chloride solution and then partitioned between ethyl acetate and water. The organic layer was washed with brine, dried over Na₂SO₄, filtered and concentrated under reduced pressure to afford a residue. The crude product was purified by ISCO Combiflash chromatography by eluting with 5-20% methanol in chloroform to afford methyl (S)-(7-((1-((tert-butyldiphenylsilyl)oxy)hexan-3-yl)amino)-1-((5-(hydroxymethyl)-3-methoxypyridin-2-yl)methyl)-3-methyl-1H-pyrazolo[4,3-d]pyrimidin-5-yl)carbamate (358 mg, 0.357 mmol, 67.3% yield) as a light brown solid.

LC-MS m/z 712.3 [M+H]⁺.

Step 8. To a stirred solution of methyl (S)-(7-((1-((tert-butyldiphenylsilyl)oxy)hexan-3-yl)amino)-1-((5-(hydroxymethyl)-3-methoxypyridin-2-yl)methyl)-3-methyl-1H-pyrazolo[4,3-d]pyrimidin-5-yl)carbamate (0.1 g, 0.140 mmol) in THF (3.0 mL), was added SOCl₂ (0.2 mL, 2.74 mmol) at 0° C. The reaction mixture was stirred at 0° C. for 30 min. The mixture was concentrated under reduced pressure to afford methyl (S)-(7-((1-((tert-butyldiphenyl-silyl)oxy)hexan-3-yl)amino)-1-((5-(chloromethyl)-3-methoxypyridin-2-yl)methyl)-3-methyl-1H-pyrazolo[4,3-d]pyrimidin-5-yl)carbamate (0.118 g, assumed 100% yield) as a light brown solid. The crude product was used as such in the next step.

LC-MS m/z 730.2 [M+H]⁺.

Step 9. To a stirred solution of methyl (S)-(7-((1-((tert-butyldiphenylsilyl)oxy)hexan-3-yl)amino)-1-((5-(chloromethyl)-3-methoxypyridin-2-yl)methyl)-3-methyl-1H-pyrazolo[4,3-d]pyrimidin-5-yl)carbamate (0.11 g, 0.151 mmol) in acetonitrile (3.0 mL), were added tetrahydro-2H-pyran-4-amine hydrochloride (0.031 g, 0.226 mmol), Na₂CO₃ (0.048 g, 0.452 mmol) and KI (0.025 g, 0.151 mmol). The reaction mixture was stirred at 50° C. for 16 h and then filtered through a CELITE™ bed and washed with excess EtOAc. The filtrate was concentrated under reduced pressure to afford the residue, which was triturated with diethyl ether and petroleum ether. After decanting the solvent, the residue was dried under vacuum to afford methyl (S)-(7-((1-((tert-butyldiphenylsilyl)oxy)hexan-3-yl)amino)-1-((3-methoxy-5-(((tetrahydro-2H-pyran-4-yl)amino)methyl)pyridin-2-yl)methyl)-3-methyl-1H-pyrazolo[4,3-d]pyrimidin-5-yl)carbamate (206 mg, 0.018 mmol, 12.04% yield) as a brown semi-solid.

LC-MS m/z 795.4 [M+H]⁺.

Step 10. To a stirred solution of methyl (S)-(7-((1-((tert-butyldiphenylsilyl)oxy)hexan-3-yl)amino)-1-((3-methoxy-5-(((tetrahydro-2H-pyran-4-yl)amino)methyl)pyridin-2-yl)methyl)-3-methyl-1H-pyrazolo[4,3-d]pyrimidin-5-yl)carbamate (0.2 g, 0.252 mmol) in MeOH (2.0 mL), concentrated HCl (1.0 mL, 1.500 mmol) was added. The reaction mixture was stirred at RT for 2 h. The mixture was concentrated under reduced pressure to afford a residue, which was triturated with diethyl ether and petroleum ether. After decanting the solvent, the residue was dried under vacuum to afford methyl (S)-(7-((1-hydroxyhexan-3-yl)amino)-1-((3-methoxy-5-(((tetrahydro-2H-pyran-4-yl)amino)methyl)pyridin-2-yl)methyl)-3-methyl-1H-pyrazolo[4,3-d]pyrimidin-5-yl)carbamate (209 mg, 0.086 mmol, 34.3% yield) as a brown solid.

LC-MS m/z 557.4 [M+H]⁺.

Step 11. To a stirred solution of methyl (S)-(7-((1-hydroxyhexan-3-yl)amino)-1-((3-methoxy-5-(((tetrahydro-2H-pyran-4-yl)amino)methyl)pyridin-2-yl)methyl)-3-methyl-1H-pyrazolo[4,3-d]pyrimidin-5-yl)carbamate (0.2 g, 0.359 mmol) in 1,4-dioxane (2.0 mL), NaOH (1.0 mL, 0.359 mmol) was added. The reaction mixture was stirred at 80° C. for 2 h and cooled to RT. The organic layer was separated, dried over Na₂SO₄, filtered and concentrated under reduced pressure to afford a residue. The crude product was purified by reversed phase preparative LC/MS (Column: Waters XBridge C18, 19×150 mm, 5-μm particles; mobile phase A: 10 mM NH₄OAc; mobile phase B: acetonitrile; gradient: 10-45% B over 20 minutes, then a 5-minute hold at 100% B; flow rate: 15 mL/min). Fraction collection was triggered by MS and UV signals. Fractions containing the desired product were combined and dried via centrifugal evaporation using a Genevac apparatus to afford Compound 134 (27.2 mg, 0.051 mmol, 14.07% yield).

### Example 18—Compound 135

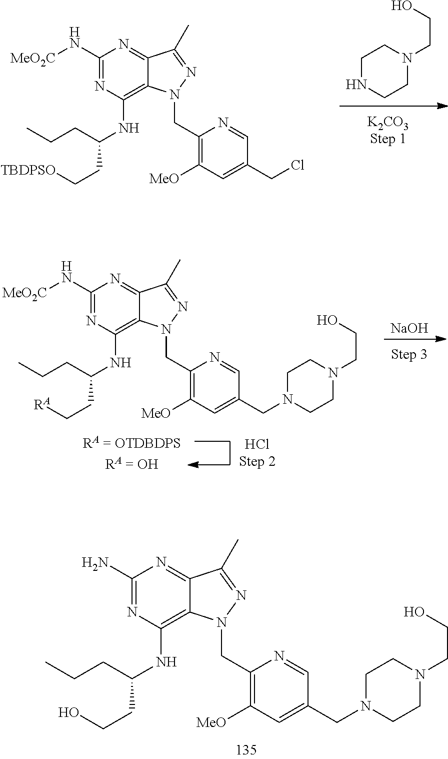

Step 1. To a stirred solution of methyl (S)-(7-((1-((tert-butyldiphenylsilyl)oxy)hexan-3-yl)amino)-1-((5-(chloromethyl)-3-methoxypyridin-2-yl)methyl)-3-methyl-1H-pyrazolo[4,3-d]pyrimidin-5-yl)carbamate (0.25 g, 0.342 mmol) in DMF (3.0 mL), were added 2-(piperazin-1-yl)ethan-1-ol (0.089 g, 0.685 mmol) and K₂CO₃ (0.142 g, 1.027 mmol). The reaction mixture was stirred at 45° C. for 3 h. The mixture was filtered through a CELITE™ bed, which was subsequently washed with excess of ethyl acetate. The filtrate was concentrated under reduced pressure to afford methyl (S)-(7-((1-((tert-butyldiphenylsilyl)oxy)hexan-3-yl)amino)-1-((5-((4-(2-hydroxyethyl)piperazin-1-yl)methyl)-3-methoxypyridin-2-yl)methyl)-3-methyl-1H-pyrazolo[4,3-d]pyrimidin-5-yl)carbamate (252 mg, 0.260 mmol, 76% yield) as a brown solid.

LC-MS m/z 824.4 [M+H]⁺.

Step 2. To a stirred solution of methyl (S)-(7-((1-((tert-butyldiphenylsilyl)oxy)hexan-3-yl)amino)-1-((5-((4-(2-hydroxyethyl)piperazin-1-yl)methyl)-3-methoxypyridin-2-yl)methyl)-3-methyl-1H-pyrazolo[4,3-d]pyrimidin-5-yl)carbamate (200.0 mg, 0.243 mmol) in MeOH (2.0 mL), conc. HCl (1.5 N HCl in water) (2.0 mL, 3.00 mmol) was added. The reaction mixture was stirred at RT for 1 h. The reaction mixture was concentrated under reduced pressure to afford the residue, which was triturated with diethyl ether and petroleum ether. The precipitated solid was dried under vacuum to afford methyl (S)-(1-((5-((4-(2-hydroxyethyl)piperazin-1-yl)methyl)-3-methoxypyridin-2-yl)methyl)-7-((1-hydroxyhexan-3-yl)amino)-3-methyl-1H-pyrazolo[4,3-d]pyrimidin-5-yl)carbamate (210 mg, 0.201 mmol, 83% yield).

LC-MS m/z 586.4 [M+H]⁺.

Step 3. To a stirred solution of methyl (S)-(1-((5-((4-(2-hydroxyethyl)piperazin-1-yl)methyl)-3-methoxypyridin-2-yl)methyl)-7-((1-hydroxyhexan-3-yl)amino)-3-methyl-1H-pyrazolo[4,3-d]pyrimidin-5-yl)carbamate (0.25 g, 0.427 mmol) in 1,4-dioxane (2.0 mL), NaOH (2.5 mL, 0.427 mmol) was added. The reaction mixture was stirred at 70° C. for 2 h and cooled to RT. The organic layer was separated, dried over Na₂SO₄, filtered and concentrated under reduced pressure to afford a residue. The residue was purified by reversed phase preparative LC/MS (Column: Waters XBridge C18, 19×150 mm, 5-μm particles; mobile phase A: 10-mM NH₄OAc; mobile phase B: acetonitrile; gradient: 20-75% B over 20 minutes, then a 5-minute hold at 100% B; flow rate: 15 mL/min). Fraction collection was triggered by MS and UV signals. Fractions containing the desired product were combined and dried via centrifugal evaporation using Genevac apparatus to afford Compound 135 (37.0 mg, 0.070 mmol, 16.43% yield).

### Example 19—Compound 137

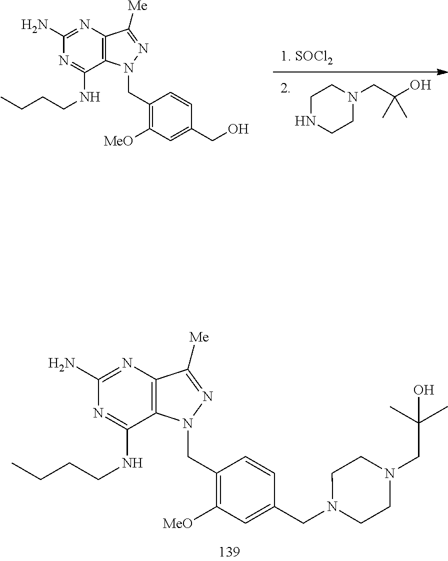 [SMILES: CCCCNc1nc(N)nc2c(C)nn(Cc3ccc(CO)cc3OC)c12 ; "1. SOCl2" ; CC(C)(O)CN1CCNCC1 ; CCCCNc1nc(N)nc2c(C)nn(Cc3ccc(CN4CCN(CC(C)(C)O)CC4)cc3OC)c12]

A solution of (4-((5-amino-7-(butylamino)-3-methyl-1H-pyrazolo[4,3-d]pyrimidin-1-yl)methyl)-3-methoxyphenyl)methanol (30 mg, 0.081 mmol) in THF (1 mL) was treated with SOCl₂ (0.012 mL, 0.162 mmol) and stirred at RT for 30 min. The solvent was evaporated in a V-10 evaporator. The crude chloride was dissolved in DMSO (1 mL). The reaction mixture was treated with 2-methyl-1-(piperazin-1-yl)propan-2-ol (25.6 mg, 0.162 mmol) and heated at 70° C. for 2 h. The crude material was purified via preparative LC/MS with the following conditions: Column: XBridge C18, 200 mm×19 mm, 5 am particles; Mobile Phase A: 5:95 acetonitrile:water with NH₄OAc; Mobile Phase B: 95:5 acetonitrile:water with NH₄OAc; Gradient: a 0-minute hold at 14% B, 14-54% B over 20 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile:water with 0.05% TFA; Mobile Phase B: 95:5 acetonitrile:water with 0.05% TFA; Gradient: a 0-minute hold at 0% B, 0-40% B over 20 minutes, then a 0-minute hold at 100% B; Flow Rate: 20 mL/min; Column Temperature: 25° C. Fractions containing Compound 137, collection triggered by MS signals, were combined and dried via centrifugal evaporation.

### Example 20—Starting Materials and Intermediates

The Charts below show schemes for making compounds that could be useful as starting materials or intermediates for the preparation of TLR7 agonists disclosed herein. The schemes can be adapted to make other, analogous compounds that could be used as starting materials or intermediates. The reagents employed are well known in the art and in many instances their use has been demonstrated in the preceding Examples.

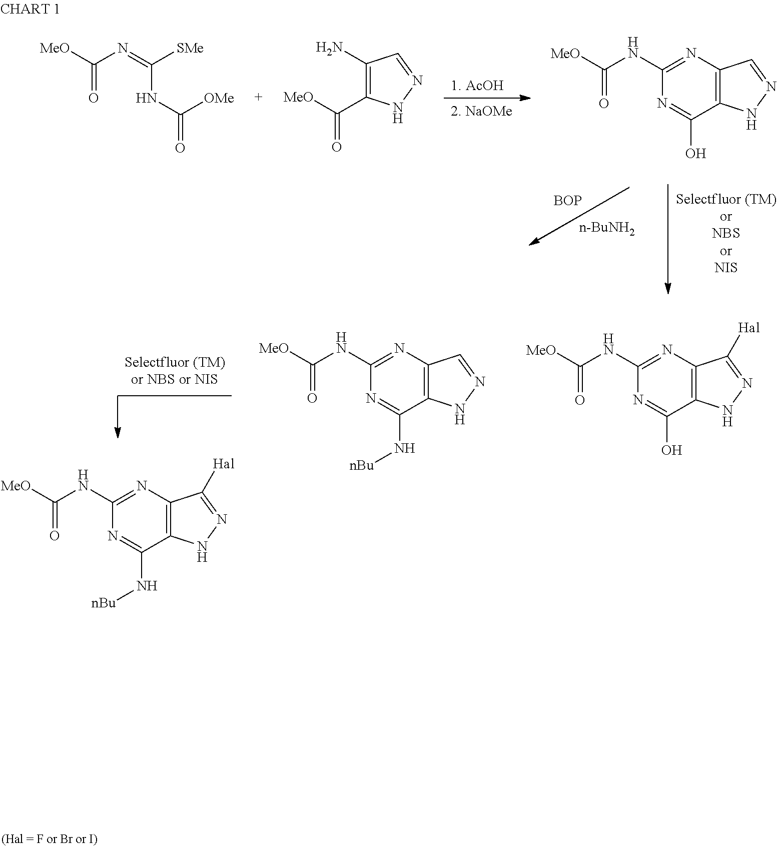

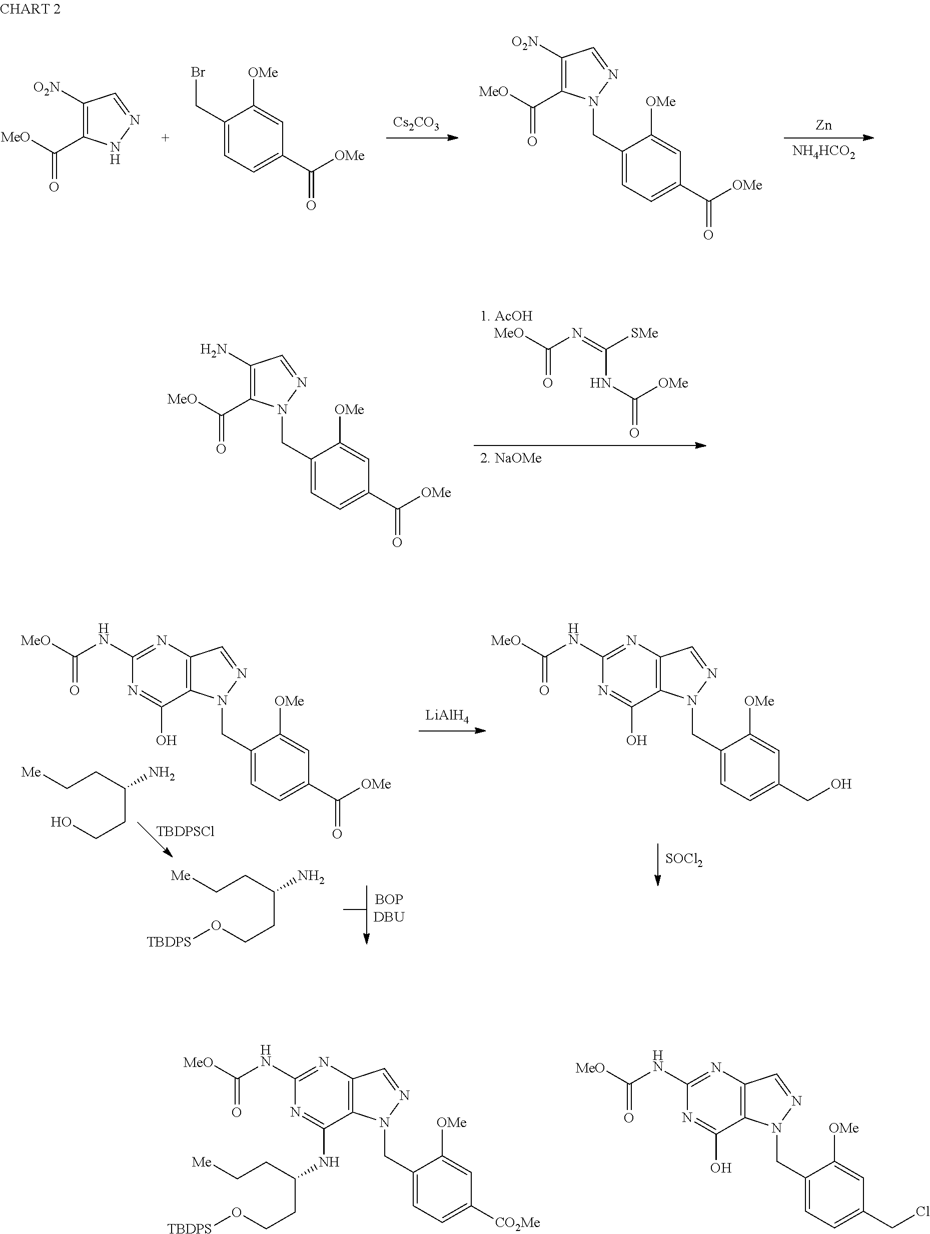

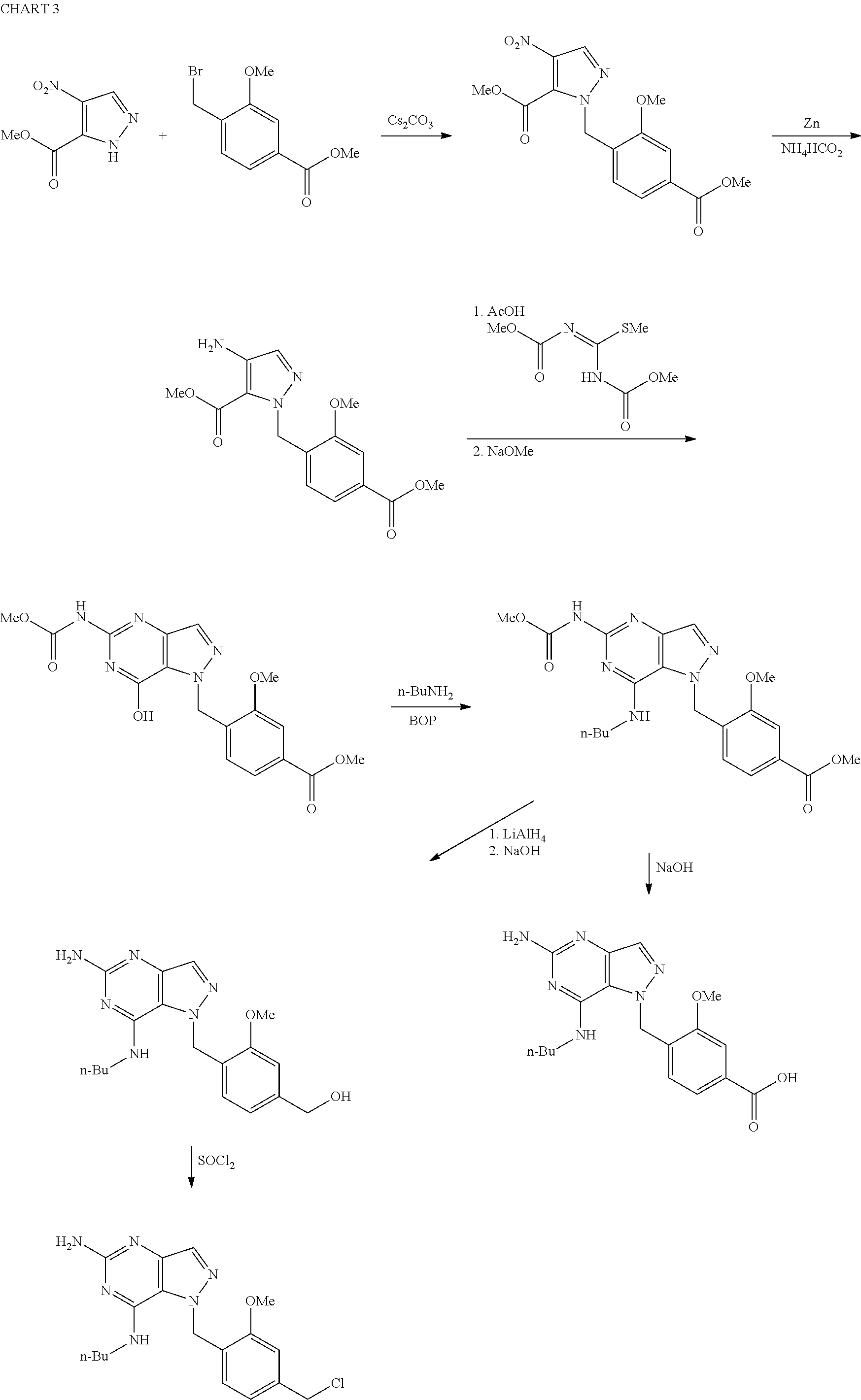

### Biological Activity

The biological activity of compounds disclosed herein as TLR7 agonists can be assayed by the procedures following.

### Human TLR7 Agonist Activity Assay

This procedure describes a method for assaying human TLR7 (hTLR7) agonist activity of the compounds disclosed in this specification.

Engineered human embryonic kidney blue cells (HEK-Blue™ TLR cells; Invivogen) possessing a human TLR7-secreted embryonic alkaline phosphatase (SEAP) reporter transgene were suspended in a non-selective, culture medium (DMEM high-glucose (Invitrogen), supplemented with 10% fetal bovine serum (Sigma)). HEK-Blue™ TLR7 cells were added to each well of a 384-well tissue-culture plate (15,000 cells per well) and incubated 16-18 h at 37° C., 5% CO₂. Compounds (100 nl) were dispensed into wells containing the HEK-Blue™ TLR cells and the treated cells were incubated at 37° C., 5% CO₂. After 18 h treatment ten microliters of freshly-prepared Quanti-Blue™ reagent (Invivogen) was added to each well, incubated for 30 min (37° C., 5% CO₂) and SEAP levels measured using an Envision plate reader (OD=620 nm). The half maximal effective concentration values (EC₅₀; compound concentration which induced a response halfway between the assay baseline and maximum) were calculated.

### Induction of Type I Interferon Genes (MX-1) and CD69 in Human Blood

The induction of Type I interferon (IFN) MX-1 genes and the B-cell activation marker CD69 are downstream events that occur upon activation of the TLR7 pathway. The following is a human whole blood assay that measures their induction in response to a TLR7 agonist.

Heparinized human whole blood was harvested from human subjects and treated with test TLR7 agonist compounds at 1 mM. The blood was diluted with RPMI 1640 media and Echo was used to predot 10 nL per well giving a final concentration of 1 uM (10 nL in 10 uL of blood). After mixing on a shaker for 30 sec, the plates were covered and placed in a 37° C. chamber for o/n=17 hrs. Fixing/lysis buffer was prepared (5×->1× in H₂O, warm at 37° C.; Cat #BD 558049) and kept the perm buffer (on ice) for later use.

For surface markers staining (CD69): prepared surface Abs: 0.045 ul hCD14-FITC (ThermoFisher Cat #MHCD1401)+0.6 ul hCD19-ef450 (ThermoFisher Cat #48-0198-42)+1.5 ul hCD69-PE (cat #BD555531)+0.855 ul FACS buffer. Added 3 ul/well, spin 1000 rpm for 1 min and mixed on shaker for 30 sec, put on ice for 30 mins. Stop stimulation after 30 minutes with 70 uL of prewarmed 1× fix/lysis buffer and use Feliex mate to resuspend (15 times, change tips for each plate) and incubate at 37 C for 10 minutes.

Centrifuge at 2000 rpm for 5 minutes aspirate with HCS plate washer, mix on shaker for 30 sec and then wash with 70 uL in dPBS and pelleted 2×s (2000 rpm for 5 min) and 50 ul wash in FACS buffer pelleted 1×s (2000 rpm for 5 min). Mix on shaker for 30 sec. For Intracellular markers staining (MX-1): Add 50 ul of BD Perm buffer III and mix on shaker for 30 sec. Incubate on ice for 30 minutes (in the dark). Wash with 50 uL of FACS buffer 2× (spin @2300 rpm×5 min after perm) followed by mixing on shaker for 30 sec. Resuspended in 20 ul of FACS buffer containing MX1 antibody ( )(4812)-Alexa 647: Novus Biologicals #NBP2-43704AF647) 20 ul FACS bf+0.8 ul hIgG+0.04 ul MX-1. Spin 1000 rpm for 1 min, mix on shaker for 30 se and the samples were incubated at RT in the dark for 45 minutes followed by washing 2×FACS buffer (spin @2300 rpm×5 min after perm). Resuspend 20 ul (35 uL total per well) of FACS buffer and cover with foil and place in 4° C. to read the following day. Plates were read on iQuePlus. The results were loaded into toolset and IC50 curves are generated in curve master. The y-axis 100% is set to 1 uM of resiquimod.

### Induction of TNF-Alpha and Type I IFN Response Genes in Mouse Blood

The induction of TNF-alpha and Type I IFN response genes are downstream events that occur upon activation of the TLR7 pathway. The following is an assay that measures their induction in whole mouse blood in response to a TLR7 agonist.

Heparinized mouse whole blood was diluted with RPMI 1640 media with Pen-Strep in the ratio of 5:4 (50 uL whole blood and 40 uL of media). A volume of 90 uL of the diluted blood was transferred to wells of Falcon flat bottom 96-well tissue culture plates, and the plates were incubated at 4° C. for 1 h. Test compounds in 100% DMSO stocks were diluted 20-fold in the same media for concentration response assays, and then 10 uL of the diluted test compounds were added to the wells, so that the final DMSO concentration was 0.5%. Control wells received 10 uL media containing 5% DMSO. The plates were then incubated at 37° C. in a 5% CO₂ incubator for 17 h. Following the incubation, 100 uL of the culture medium as added to each well. The plates were centrifuged and 130 uL of supernatant was removed for use in assays of TNFa production by ELISA (Invitrogen, Catalog Number 88-7324 by Thermo-Fisher Scientific). A 70 uL volume of mRNA catcher lysis buffer (1×) with DTT from the Invitrogen mRNA Catcher Plus kit (Cat #K1570-02) was added to the remaining 70 uL sample in the well, and was mixed by pipetting up and down 5 times. The plate was then shaken at RT for 5-10 min, followed by addition of 2 uL of proteinase K (20 mg/mL) to each well. Plates were then shaken for 15-20 min at RT. The plates were then stored at −80° C. until further processing.

The frozen samples were thawed and mRNA was extracted using the Invitrogen mRNA Catcher Plus kit (Cat #K1570-02) according to the manufacturer's instructions. Half yield of mRNA from RNA extraction were used to synthesize cDNA in 20 μL reverse transcriptase reactions using Invitrogen SuperScript IV VILO Master Mix (Cat #11756500). TaqMan® real-time PCR was performed using QuantStudio Real-Time PCR system from ThermoFisher (Applied Biosystems). All real-time PCR reactions were run in duplicate using commercial predesigned TaqMan assays for mouse IFIT1, IFIT3, MX1 and PPIA gene expression and TaqMan Master Mix. PPIA was utilized as the housekeeping gene. The recommendations from the manufacturer were followed. All raw data (Ct) were normalized by average housekeeping gene (Ct) and then the comparative Ct (ΔΔCt) method were utilized to quantify relative gene expression (RQ) for experimental analysis.

### Definitions

“Aliphatic” means a straight- or branched-chain, saturated or unsaturated, non-aromatic hydrocarbon moiety having the specified number of carbon atoms (e.g., as in “C₃ aliphatic,” “C₁₋₅ aliphatic,” “C₁-C₅ aliphatic,” or “C₁ to C₅ aliphatic,” the latter three phrases being synonymous for an aliphatic moiety having from 1 to 5 carbon atoms) or, where the number of carbon atoms is not explicitly specified, from 1 to 4 carbon atoms (2 to 4 carbons in the instance of unsaturated aliphatic moieties). A similar understanding is applied to the number of carbons in other types, as in C₂₋₄ alkene, C₄-C₇ cycloaliphatic, etc. In a similar vein, a term such as “(CH₂)₁₋₃” is to be understand as shorthand for the subscript being 1, 2, or 3, so that such term represents CH₂, CH₂CH₂, and CH₂CH₂CH₂.

“Alkyl” means a saturated aliphatic moiety, with the same convention for designating the number of carbon atoms being applicable. By way of illustration, C₁-C₄ alkyl moieties include, but are not limited to, methyl, ethyl, propyl, isopropyl, isobutyl, t-butyl, 1-butyl, 2-butyl, and the like. “Alkanediyl” (sometimes also referred to as “alkylene”) means a divalent counterpart of an alkyl group, such as

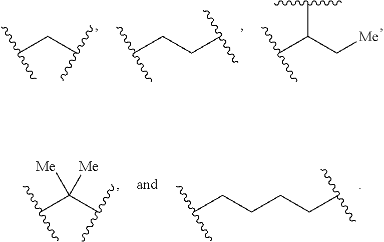

“Alkenyl” means an aliphatic moiety having at least one carbon-carbon double bond, with the same convention for designating the number of carbon atoms being applicable. By way of illustration, C₂-C₄ alkenyl moieties include, but are not limited to, ethenyl (vinyl), 2-propenyl (allyl or prop-2-enyl), cis-1-propenyl, trans-1-propenyl, E- (or Z-) 2-butenyl, 3-butenyl, 1,3-butadienyl (but-1,3-dienyl) and the like.

“Alkynyl” means an aliphatic moiety having at least one carbon-carbon triple bond, with the same convention for designating the number of carbon atoms being applicable. By way of illustration, C₂-C₄ alkynyl groups include ethynyl (acetylenyl), propargyl (prop-2-ynyl), 1-propynyl, but-2-ynyl, and the like.

“Cycloaliphatic” means a saturated or unsaturated, non-aromatic hydrocarbon moiety having from 1 to 3 rings, each ring having from 3 to 8 (preferably from 3 to 6) carbon atoms. “Cycloalkyl” means a cycloaliphatic moiety in which each ring is saturated. “Cycloalkenyl” means a cycloaliphatic moiety in which at least one ring has at least one carbon-carbon double bond. “Cycloalkynyl” means a cycloaliphatic moiety in which at least one ring has at least one carbon-carbon triple bond. By way of illustration, cycloaliphatic moieties include, but are not limited to, cyclopropyl, cyclobutyl, cyclopentyl, cyclopentenyl, cyclohexyl, cyclohexenyl, cycloheptyl, cyclooctyl, and adamantyl. Preferred cycloaliphatic moieties are cycloalkyl ones, especially cyclopropyl, cyclobutyl, cyclopentyl, and cyclohexyl. “Cycloalkanediyl” (sometimes also referred to as “cycloalkylene”) means a divalent counterpart of a cycloalkyl group. Similarly, “bicycloalkanediyl” (osr “bicycloalkylene”) and “spiroalkanediyl” (or “spiroalkylene”) refer to divalent counterparts of a bicycloalkyl and spiroalkyl (or “spirocycloalkyl”) group.

“Heterocycloaliphatic” means a cycloaliphatic moiety wherein, in at least one ring thereof, up to three (preferably 1 to 2) carbons have been replaced with a heteroatom independently selected from N, O, or S, where the N and S optionally may be oxidized and the N optionally may be quaternized. Preferred cycloaliphatic moieties consist of one ring, 5- to 6-membered in size. Similarly, “heterocycloalkyl,” “heterocycloalkenyl,” and “heterocycloalkynyl” means a cycloalkyl, cycloalkenyl, or cycloalkynyl moiety, respectively, in which at least one ring thereof has been so modified. Exemplary heterocycloaliphatic moieties include aziridinyl, azetidinyl, 1,3-dioxanyl, oxetanyl, tetrahydrofuryl, pyrrolidinyl, piperidinyl, piperazinyl, tetrahydropyranyl, tetrahydrothiopyranyl, tetrahydrothiopyranyl sulfone, morpholinyl, thiomorpholinyl, thiomorpholinyl sulfoxide, thiomorpholinyl sulfone, 1,3-dioxolanyl, tetrahydro-1,1-dioxothienyl, 1,4-dioxanyl, thietanyl, and the like. “Heterocycloalkylene” means a divalent counterpart of a heterocycloalkyl group.

“Alkoxy,” “aryloxy,” “alkylthio,” and “arylthio” mean —O(alkyl), —O(aryl), —S(alkyl), and —S(aryl), respectively. Examples are methoxy, phenoxy, methylthio, and phenylthio, respectively.

“Halogen” or “halo” means fluorine, chlorine, bromine or iodine, unless a narrower meaning is indicated.

“Aryl” means a hydrocarbon moiety having a mono-, bi-, or tricyclic ring system (preferably monocyclic) wherein each ring has from 3 to 7 carbon atoms and at least one ring is aromatic. The rings in the ring system may be fused to each other (as in naphthyl) or bonded to each other (as in biphenyl) and may be fused or bonded to non-aromatic rings (as in indanyl or cyclohexylphenyl). By way of further illustration, aryl moieties include, but are not limited to, phenyl, naphthyl, tetrahydronaphthyl, indanyl, biphenyl, phenanthryl, anthracenyl, and acenaphthyl. “Arylene” means a divalent counterpart of an aryl group, for example 1,2-phenylene, 1,3-phenylene, or 1,4-phenylene.

“Heteroaryl” means a moiety having a mono-, bi-, or tricyclic ring system (preferably 5- to 7-membered monocyclic) wherein each ring has from 3 to 7 carbon atoms and at least one ring is an aromatic ring containing from 1 to 4 heteroatoms independently selected from N, O, or S, where the N and S optionally may be oxidized and the N optionally may be quaternized. Such at least one heteroatom containing aromatic ring may be fused to other types of rings (as in benzofuranyl or tetrahydroisoquinolyl) or directly bonded to other types of rings (as in phenylpyridyl or 2-cyclopentylpyridyl). By way of further illustration, heteroaryl moieties include pyrrolyl, furanyl, thiophenyl (thienyl), imidazolyl, pyrazolyl, oxazolyl, isoxazolyl, thiazolyl, isothiazolyl, triazolyl, tetrazolyl, pyridyl, N-oxopyridyl, pyridazinyl, pyrimidinyl, pyrazinyl, quinolinyl, isoquinolynyl, quinazolinyl, cinnolinyl, quinozalinyl, naphthyridinyl, benzofuranyl, indolyl, benzothiophenyl, oxadiazolyl, thiadiazolyl, phenothiazolyl, benzimidazolyl, benzotriazolyl, dibenzofuranyl, carbazolyl, dibenzothiophenyl, acridinyl, and the like. “Heteroarylene” means a divalent counterpart of a heteroaryl group.

Where it is indicated that a moiety may be substituted, such as by use of “unsubstituted or substituted” or “optionally substituted” phrasing as in “unsubstituted or substituted C₁-C₅ alkyl” or “optionally substituted heteroaryl,” such moiety may have one or more independently selected substituents, preferably one to five in number, more preferably one or two in number. Substituents and substitution patterns can be selected by one of ordinary skill in the art, having regard for the moiety to which the substituent is attached, to provide compounds that are chemically stable and that can be synthesized by techniques known in the art as well as the methods set forth herein. Where a moiety is identified as being “unsubstituted or substituted” or “optionally substituted,” in a preferred embodiment such moiety is unsubstituted.

“Arylalkyl,” (heterocycloaliphatic)alkyl,” “arylalkenyl,” “arylalkynyl,” “biarylalkyl,” and the like mean an alkyl, alkenyl, or alkynyl moiety, as the case may be, substituted with an aryl, heterocycloaliphatic, biaryl, etc., moiety, as the case may be, with the open (unsatisfied) valence at the alkyl, alkenyl, or alkynyl moiety, for example as in benzyl, phenethyl, N-imidazoylethyl, N-morpholinoethyl, and the like. Conversely, “alkylaryl,” “alkenylcycloalkyl,” and the like mean an aryl, cycloalkyl, etc., moiety, as the case may be, substituted with an alkyl, alkenyl, etc., moiety, as the case may be, for example as in methylphenyl (tolyl) or allylcyclohexyl. “Hydroxyalkyl,” “haloalkyl,” “alkylaryl,” “cyanoaryl,” and the like mean an alkyl, aryl, etc., moiety, as the case may be, substituted with one or more of the identified substituent (hydroxyl, halo, etc., as the case may be).

For example, permissible substituents include, but are not limited to, alkyl (especially methyl or ethyl), alkenyl (especially allyl), alkynyl, aryl, heteroaryl, cycloaliphatic, heterocycloaliphatic, halo (especially fluoro), haloalkyl (especially trifluoromethyl), hydroxyl, hydroxyalkyl (especially hydroxyethyl), cyano, nitro, alkoxy, —O(hydroxyalkyl), —O(haloalkyl) (especially —OCF₃), —O(cycloalkyl), —O(heterocycloalkyl), —O(aryl), alkylthio, arylthio, ═O, ═NH, ═N(alkyl), ═NOH, ═NO(alkyl), —C(═O)(alkyl), —C(═O)H, —CO₂H, —C(═O)NHOH, —C(═O)O(alkyl), —C(═O)O(hydroxyalkyl), —C(═O)NH₂, —C(═O)NH(alkyl), —C(═O)N(alkyl)₂, —OC(═O)(alkyl), —OC(═O)(hydroxyalkyl), —OC(═O)O(alkyl), —OC(═O)O(hydroxyalkyl), —OC(═O)NH₂, —OC(═O)NH(alkyl), —OC(═O)N(alkyl)₂, azido, —NH₂, —NH(alkyl), —N(alkyl)₂, —NH(aryl), —NH(hydroxyalkyl), —NHC(═O)(alkyl), —NHC(═O)H, —N HC(═O)NH₂, —NHC(═O)NH(alkyl), —NHC(═O)N(alkyl)₂, —NHC(═NH)NH₂, —OSO₂(alkyl), —SH, —S(alkyl), —S(aryl), —S(cycloalkyl), —S(═O)alkyl, —SO₂(alkyl), —SO₂NH₂, —SO₂NH(alkyl), —SO₂N(alkyl)₂, and the like.

Where the moiety being substituted is an aliphatic moiety, preferred substituents are aryl, heteroaryl, cycloaliphatic, heterocycloaliphatic, halo, hydroxyl, cyano, nitro, alkoxy, —O(hydroxyalkyl), —O(haloalkyl), —O(cycloalkyl), —O(heterocycloalkyl), —O(aryl), alkylthio, arylthio, ═O, ═NH, ═N(alkyl), ═NOH, ═NO(alkyl), —CO₂H, —C(═O)NHOH, —C(═O)O(alkyl), —C(═O)O(hydroxyalkyl), —C(═O)NH₂, —C(═O)NH(alkyl), —C(═O)N(alkyl)₂, —OC(═O)(alkyl), —OC(═O)(hydroxyalkyl), —OC(═O)O(alkyl), —OC(═O)O(hydroxyalkyl), —OC(═O)NH₂, —OC(═O)NH(alkyl), —OC(═O)N(alkyl)₂, azido, —NH₂, —NH(alkyl), —N(alkyl)₂, —NH(aryl), —NH(hydroxyalkyl), —NHC(═O)(alkyl), —NHC(═O)H, —N HC(═O)NH₂, —NHC(═O)NH(alkyl), —NHC(═O)N(alkyl)₂, —NHC(═NH)NH₂, —OSO₂(alkyl), —SH, —S(alkyl), —S(aryl), —S(═O)alkyl, —S(cycloalkyl), —SO₂(alkyl), —SO₂NH₂, —SO₂NH(alkyl), and —SO₂N(alkyl)₂. More preferred substituents are halo, hydroxyl, cyano, nitro, alkoxy, —O(aryl), ═O, ═NOH, ═NO(alkyl), —OC(═O)(alkyl), —OC(═O)O(alkyl), —OC(═O)NH₂, —OC(═O)NH(alkyl), —OC(═O)N(alkyl)₂, azido, —NH₂, —NH(alkyl), —N(alkyl)₂, —NH(aryl), —NHC(═O)(alkyl), —NHC(═O)H, —NHC(═O)NH₂, —NHC(═O)NH(alkyl), —NHC(═O)N(alkyl)₂, and —NHC(═NH)NH₂. Especially preferred are phenyl, cyano, halo, hydroxyl, nitro, C₁-C₄ alkyoxy, O(C₂-C₄ alkanediyl)OH, and O(C₂-C₄ alkanediyl)halo.

Where the moiety being substituted is a cycloaliphatic, heterocycloaliphatic, aryl, or heteroaryl moiety, preferred substituents are alkyl, alkenyl, alkynyl, halo, haloalkyl, hydroxyl, hydroxyalkyl, cyano, nitro, alkoxy, —O(hydroxyalkyl), —O(haloalkyl), —O(aryl), —O(cycloalkyl), —O(heterocycloalkyl), alkylthio, arylthio, —C(═O)(alkyl), —C(═O)H, —CO₂H, —C(═O)NHOH, —C(═O)O(alkyl), —C(═O)O(hydroxyalkyl), —C(═O)NH₂, —C(═O)NH(alkyl), —C(═O)N(alkyl)₂, —OC(═O)(alkyl), —OC(═O)(hydroxyalkyl), —OC(═O)O(alkyl), —OC(═O)O(hydroxyalkyl), —OC(═O)NH₂, —OC(═O)NH(alkyl), —OC(═O)N(alkyl)₂, azido, —NH₂, —NH(alkyl), —N(alkyl)₂, —NH(aryl), —NH(hydroxyalkyl), —NHC(═O)(alkyl), —NHC(═O)H, —N HC(═O)NH₂, —NHC(═O)NH(alkyl), —NHC(═O)N(alkyl)₂, —NHC(═NH)NH₂, —OSO₂(alkyl), —SH, —S(alkyl), —S(aryl), —S(cycloalkyl), —S(═O)alkyl, —SO₂(alkyl), —SO₂NH₂, —SO₂NH(alkyl), and —SO₂N(alkyl)₂. More preferred substituents are alkyl, alkenyl, halo, haloalkyl, hydroxyl, hydroxyalkyl, cyano, nitro, alkoxy, —O(hydroxyalkyl), —C(═O)(alkyl), —C(═O)H, —CO₂H, —C(═O)NHOH, —C(═O)O(alkyl), —C(═O)O(hydroxyalkyl), —C(═O)NH₂, —C(═O)NH(alkyl), —C(═O)N(alkyl)₂, —OC(═O)(alkyl), —OC(═O)(hydroxyalkyl), —OC(═O)O(alkyl), —OC(═O)O(hydroxyalkyl), —OC(═O)NH₂, —OC(═O)NH(alkyl), —OC(═O)N(alkyl)₂, —NH₂, —NH(alkyl), —N(alkyl)₂, —NH(aryl), —NHC(═O)(alkyl), —NHC(═O)H, —NHC(═O)NH₂, —NHC(═O)NH(alkyl), —NHC(═O)N(alkyl)₂, and —NHC(═NH)NH₂. Especially preferred are C₁-C₄ alkyl, cyano, nitro, halo, and C₁-C₄alkoxy.

Where a range is stated, as in “C₁-C₅ alkyl” or “5 to 10%,” such range includes the end points of the range, as in C₁ and C₅ in the first instance and 5% and 10% in the second instance.

Unless particular stereoisomers are specifically indicated (e.g., by a bolded or dashed bond at a relevant stereocenter in a structural formula, by depiction of a double bond as having E or Z configuration in a structural formula, or by use stereochemistry-designating nomenclature or symbols), all stereoisomers are included within the scope of the invention, as pure compounds as well as mixtures thereof. Unless otherwise indicated, racemates, individual enantiomers (whether optically pure or partially resolved), diastereomers, geometrical isomers, and combinations and mixtures thereof are all encompassed by this invention.

Those skilled in the art will appreciate that compounds may have tautomeric forms (e.g., keto and enol forms), resonance forms, and zwitterionic forms that are equivalent to those depicted in the structural formulae used herein and that the structural formulae encompass such tautomeric, resonance, or zwitterionic forms.

“Pharmaceutically acceptable ester” means an ester that hydrolyzes in vivo (for example in the human body) to produce the parent compound or a salt thereof or has per se activity similar to that of the parent compound. Suitable esters include C₁-C₅ alkyl, C₂-C₅ alkenyl or C₂-C₅ alkynyl esters, especially methyl, ethyl or n-propyl.

“Pharmaceutically acceptable salt” means a salt of a compound suitable for pharmaceutical formulation. Where a compound has one or more basic groups, the salt can be an acid addition salt, such as a sulfate, hydrobromide, tartrate, mesylate, maleate, citrate, phosphate, acetate, pamoate (embonate), hydroiodide, nitrate, hydrochloride, lactate, methyl-sulfate, fumarate, benzoate, succinate, mesylate, lactobionate, suberate, tosylate, and the like. Where a compound has one or more acidic groups, the salt can be a salt such as a calcium salt, potassium salt, magnesium salt, meglumine salt, ammonium salt, zinc salt, piperazine salt, tromethamine salt, lithium salt, choline salt, diethylamine salt, 4-phenylcyclohexylamine salt, benzathine salt, sodium salt, tetramethylammonium salt, and the like. Polymorphic crystalline forms and solvates are also encompassed within the scope of this invention.

“Subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

The terms “treat,” “treating,” and “treatment,” in the context of treating a disease or disorder, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof. The “treatment of cancer”, refers to one or more of the following effects: (1) inhibition, to some extent, of tumor growth, including, (i) slowing down and (ii) complete growth arrest; (2) reduction in the number of tumor cells; (3) maintaining tumor size; (4) reduction in tumor size; (5) inhibition, including (i) reduction, (ii) slowing down or (iii) complete prevention, of tumor cell infiltration into peripheral organs; (6) inhibition, including (i) reduction, (ii) slowing down or (iii) complete prevention, of metastasis; (7) enhancement of anti-tumor immune response, which may result in (i) maintaining tumor size, (ii) reducing tumor size, (iii) slowing the growth of a tumor, (iv) reducing, slowing or preventing invasion and/or (8) relief, to some extent, of the severity or number of one or more symptoms associated with the disorder.

In the formulae of this specification, a wavy line ( [symbol] ) transverse to a bond or an asterisk (*) at the end of the bond denotes a covalent attachment site. For instance, a statement that R is

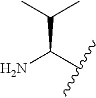 [SMILES: *[C@@H](N)C(C)C]

or that R is

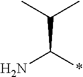 [SMILES: *[C@@H](N)C(C)C]

in the formula

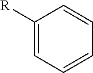

means

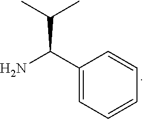

In the formulae of this specification, a bond traversing an aromatic ring between two carbons thereof means that the group attached to the bond may be located at any of the positions of the aromatic ring made available by removal of the hydrogen that is implicitly there (or explicitly there, if drawn out). By way of illustration, the formula

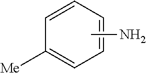

represents

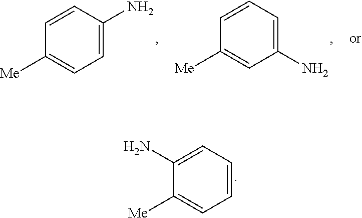 [SMILES: Cc1ccc(N)cc1 ; Cc1cccc(N)c1 ; Cc1ccccc1N]

In other illustrations,

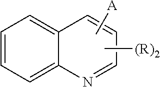

represents

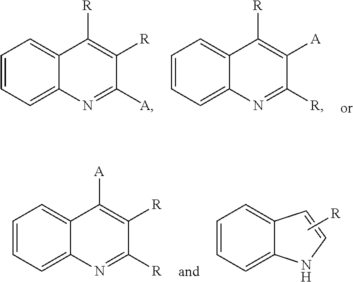

represents

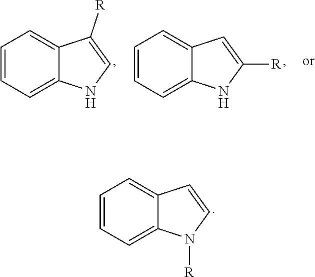

This disclosure includes all isotopes of atoms occurring in the compounds described herein. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed. By way of example, a C₁-C₃ alkyl group can be undeuterated, partially deuterated, or fully deuterated and “CH₃” includes CH₃, ¹³CH₃, ¹⁴CH₃, CH₂T, CH₂D, CHD₂, CD₃, etc. In one embodiment, the various elements in a compound are present in their natural isotopic abundance.

Those skilled in the art will appreciate that certain structures can be drawn in one tautomeric form or another—for example, keto versus enol—and that the two forms are equivalent.

### Acronyms and Abbreviations

This is a list of acronyms and abbreviations used in this specification, along with their meanings.

| ACRONYM OR ABBREVIATION | MEANING OR DEFINITION |
|---|---|
| AIBN | Azobisisobutyronitrile |
| Alloc | Allyloxycarbonyl |
| Aq. | Aqueous |
| Boc | t-Butyloxycarbonyl |
| BOP | (Benzotriazol-1-yloxy)tris(dimethylamino)- |
| | phosphonium hexafluorophosphate (V) |
| DBU | 1,8-Diazabicyclo[5.4.0]undec-7-ene |
| DCM | Dichloromethane |
| DIAD | Diisopropyl azodicarboxylate |
| DIPEA, DIEA | N,N-diisopropylethylamine, also known as |
| | Hünig's base |
| DMA | N,N-Dimethylacetamide |
| DMAP | 4-(Dimethylamino)pyridine |
| DMF | N,N-dimethylformamide |
| DMSO | Dimethyl sulfoxide |
| DTDP | 2,2′-dithiodipyridine |
| DTPA | Diethylenetriaminepentaacetic acid |
| EEDQ | Ethyl 2-ethoxyquinoline-1(2H)-carboxylate |
| Fmoc | Fluorenylmethyloxycarbonyl |
| HATU | Hexafluorophosphate Azabenzotriazole |
| | Tetramethyl Uronium; 1-[Bis(dimethylamino)- |
| | methylene]-1H-1,2,3-triazolo[4,5-b]pyridinium 3- |
| | oxide hexafluorophosphate |
| HEPES | 4-(2-Hydroxyethyl)piperazine-1-ethanesulfonic |
| | acid, N-(2-Hydroxyethyl)piperazine-N′-(2- |
| | ethanesulfonic acid) |
| HPLC | High pressure liquid chromatography |
| Hunig's base | See DIPEA, DIEA |
| LCMS, LC-MS, LC/MS | Liquid chromatography-mass spectrometry |
| mCPBA | m-chloroperbenzoic acid |
| MS | Mass spectrometry |
| MsCl | Methanesylfonyl chloride, mesyl chloride |
| NBS | N-Bromosuccinimide |
| NMR | Nuclear magnetic resonance |
| PEG | Poly(ethylene glycol) |
| PTFE | Poly(tetrafluoroethylene) |
| RT (in context of liquid | Retention time, in min |
| chromatography) | |
| RT (in the context of reaction | Room (ambient) temperature, circa 25° C. |
| conditions) | |
| Sat. | Saturated |
| Soln | Solution |
| TBDPS | tert-Butyldiphenylsilyl |
| TBS | t-Butyldimethylsilyl group |
| TEA | Triethylamine |
| TEAA | TriethylNH₄OAc |
| TFA | Trifluoroacetic acid |
| THF | Tetrahydrofuran |

### REFERENCES

Full citations for the following references cited in abbreviated fashion by first author (or inventor) and date earlier in this specification are provided below. Each of these references is incorporated herein by reference for all purposes.
Akinbobuyi et al., Tetrahedron Lett. 2015, 56, 458, “Facile syntheses of functionalized toll-like receptor 7 agonists”.
Akinbobuyi et al., Bioorg. Med. Chem. Lett. 2016, 26, 4246, “Synthesis and immunostimulatory activity of substituted TLR7 agonists.”
Barberis et al., US 2012/0003298 A1 (2012).
Beesu et al., J. Med. Chem. 2017, 60, 2084, “Identification of High-Potency Human TLR8 and Dual TLR7/TLR8 Agonists in Pyrimidine-2,4-diamines.”
Berghöfer et al., J. Immunol. 2007, 178, 4072, “Natural and Synthetic TLR7 Ligands Inhibit CpG-A- and CpG-C-Oligodeoxynucleotide-Induced IFN-α Production.”
Bonfanti et al., US 2014/0323441 A1 (2015) [2015a].
Bonfanti et al., US 2015/0299221 A1 (2015) [2015b].
Bonfanti et al., US 2016/0304531 A1 (2016).
Carson et al., US 2013/0202629 A1 (2013).
Carson et al., U.S. Pat. No. 8,729,088 B2 (2014).
Carson et al., U.S. Pat. No. 9,050,376 B2 (2015).
Carson et al., US 2016/0199499 A1 (2016).
Chan et al., Bioconjugate Chem. 2009, 20, 1194, “Synthesis and Immunological Characterization of Toll-Like Receptor 7 Agonistic Conjugates.”
Chan et al., Bioconjugate Chem. 2011, 22, 445, “Synthesis and Characterization of PEGylated Toll Like Receptor 7 Ligands.”
Chen et al., U.S. Pat. No. 7,919,498 B2 (2011).
Coe et al., U.S. Pat. No. 9,662,336 B2 (2017).
Cortez and Va, Medicinal Chem. Rev. 2018, 53, 481, “Recent Advances in Small-Molecule TLR7 Agonists for Drug Discovery”.
Cortez et al., US 2017/0121421 A1 (2017).
Cortez et al., U.S. Pat. No. 9,944,649 B2 (2018).
Dellaria et al., WO 2007/028129 A1 (2007).
Desai et al., U.S. Pat. No. 9,127,006 B2 (2015).
Ding et al., WO 2016/107536 A1 (2016).
Ding et al., US 2017/0273983 A1 (2017) [2017a].
Ding et al., WO 2017/076346 A1 (2017) [2017b].
Gadd et al., Bioconjugate Chem. 2015, 26, 1743, “Targeted Activation of Toll-Like Receptors: Conjugation of a Toll-Like Receptor 7 Agonist to a Monoclonal Antibody Maintains Antigen Binding and Specificity.”
Graupe et al., U.S. Pat. No. 8,993,755 B2 (2015).
Embrechts et al., J. Med. Chem. 2018, 61, 6236, “2,4-Diaminoquinazolines as Dual Toll Like Receptor (TLR) 7/8 Modulators for the Treatment of Hepatitis B Virus.”
Halcomb et al., U.S. Pat. No. 9,161,934 B2 (2015).
Hashimoto et al., US 2009/0118263 A1 (2009).
He et al., U.S. Pat. No. 10,487,084 B2 (2019) [2019a].
He et al., U.S. Pat. No. 10,508,115 B2 (2019) [2019b].
Hirota et al., U.S. Pat. No. 6,028,076 (2000).
Holldack et al., US 2012/0083473 A1 (2012).
Isobe et al., U.S. Pat. No. 6,376,501 B1 (2002).
Isobe et al., JP 2004137157 (2004).
Isobe et al., J. Med. Chem. 2006, 49 (6), 2088, “Synthesis and Biological Evaluation of Novel 9-Substituted-8-Hydroxyadenine Derivatives as Potent Interferon Inducers.”
Isobe et al., U.S. Pat. No. 7,521,454 B2 (2009) [2009a].
Isobe et al., US 2009/0105212 A1 (2009) [2009b].
Isobe et al., US 2011/0028715 A1 (2011).
Isobe et al., U.S. Pat. No. 8,148,371 B2 (2012).
Jensen et al., WO 2015/036044 A1 (2015).
Jones et al., U.S. Pat. No. 7,691,877 B2 (2010).
Jones et al., US 2012/0302598 A1 (2012).
Kasibhatla et al., U.S. Pat. No. 7,241,890 B2 (2007).
Koga-Yamakawa et al., Int. J. Cancer 2013, 132 (3), 580, “Intratracheal and oral administration of SM-276001: A selective TLR7 agonist, leads to antitumor efficacy in primary and metastatic models of cancer.”
Li et al., U.S. Pat. No. 9,902,730 B2 (2018).
Lioux et al., U.S. Pat. No. 9,295,732 B2 (2016).
Lund et al., Proc. Nat'l Acad. Sci (USA) 2004, 101 (15), 5598, “Recognition of single-stranded RNA viruses by Toll-like receptor 7.”
Maj et al., U.S. Pat. No. 9,173,935 B2 (2015).
McGowan et al., US 2016/0168150 A1 (2016) [2016a].
McGowan et al., U.S. Pat. No. 9,499,549 B2 (2016) [2016b].
McGowan et al., J. Med. Chem. 2017, 60, 6137, “Identification and Optimization of Pyrrolo[3,2-d]pyrimidine Toll-like Receptor 7 (TLR7) Selective Agonists for the Treatment of Hepatitis B.”
Musmuca et al., J. Chem. Information & Modeling 2009, 49 (7), 1777, “Small-Molecule Interferon Inducers. Toward the Comprehension of the Molecular Determinants through Ligand-Based Approaches.”
Nakamura et al., Bioorg. Med. Chem. Lett. 2013, 13, 669, “Synthesis and evaluation of 8-oxoadenine derivatives as potent Toll-like receptor agonists with high water solubility.”
Ogita et al., US 2007/0225303 A1 (2007).
Ota et al., WO 2019/124500 A1 (2019).
Pilatte et al., WO 2017/216293 A1 (2017).
Poudel et al., U.S. Pat. No. 10,472,361 B2 (2019) [2019a].
Poudel et al., U.S. Pat. No. 10,494,370 B2 (2019) [2019b].
Poudel et al., US 2020/0038403 A1 (2020) [2020a].
Poudel et al., US 2020/0039986 A1 (2020) [2020b].
Purandare et al., WO 2019/209811 A1 (2019).
Pryde, U.S. Pat. No. 7,642,350 B2 (2010).
Sato-Kaneko et al., JCI Insight 2017, 2, e93397, “Combination Immunotherapy with TLR Agonists and Checkpoint Inhibitors Suppresses Head and Neck Cancer”.
Smits et al., The Oncologist 2008, 13, 859, “The Use of TLR7 and TLR8 Ligands for the Enhancement of Cancer Immunotherapy”.
Vasilakos and Tomai, Expert Rev. Vaccines 2013, 12, 809, “The Use of Toll-like Receptor 7/8 Agonists as Vaccine Adjuvants”.
Vernejoul et al., US 2014/0141033 A1 (2014).
Young et al., U.S. Pat. No. 10,457,681 B2 (2019).
Yu et al., PLoS One 2013, 8 (3), e56514, “Toll-Like Receptor 7 Agonists: Chemical Feature Based Pharmacophore Identification and Molecular Docking Studies.”
Zhang et al., Immunity 2016, 45, 737, “Structural Analysis Reveals that Toll-like Receptor 7 Is a Dual Receptor for Guanosine and Single-Stranded RNA.”
Zhang et al., WO 2018/095426 A1 (2018)>
Zurawski et al., US 2012/0231023 A1 (2012).

The foregoing detailed description of the invention includes passages that are chiefly or exclusively concerned with particular parts or aspects of the invention. It is to be understood that this is for clarity and convenience, that a particular feature may be relevant in more than just the passage in which it is disclosed, and that the disclosure herein includes all the appropriate combinations of information found in the different passages. Similarly, although the various figures and descriptions herein relate to specific embodiments of the invention, it is to be understood that where a specific feature is disclosed in the context of a particular figure or embodiment, such feature can also be used, to the extent appropriate, in the context of another figure or embodiment, in combination with another feature, or in the invention in general.

Further, while the present invention has been particularly described in terms of certain preferred embodiments, the invention is not limited to such preferred embodiments. Rather, the scope of the invention is defined by the appended claims.

1. A compound having a structure according to formula I 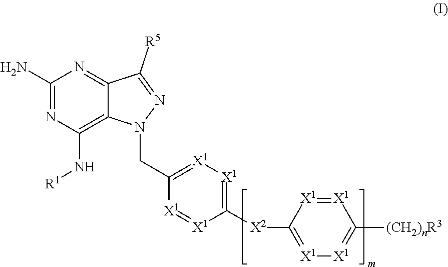
wherein
each X¹ is independently N or CR²;
X² is O, CH₂, NH, S, or N(C₁-C₃ alkyl);
R¹ is (C₁-C₅ alkyl),
(C₂-C₅ alkenyl),
(C₁-C₈ alkanediyl)₀₋₁(C₃-C₆ cycloalkyl),
(C₂-C₈ alkanediyl)OH,
(C₂-C₈ alkanediyl)O(C₁-C₃ alkyl),
(C₁-C₄ alkanediyl)₀₋₁(5-6 membered heteroaryl),
(C₁-C₄ alkanediyl)₀₋₁phenyl,
(C₁-C₄ alkanediyl)CF₃,
(C₂-C₈ alkanediyl)N[C(═O)](C₁-C₃ alkyl),
or
(C₂-C₈ alkanediyl)NR^{x}R^{y};
each R² is independently H, O(C₁-C₃ alkyl), S(C₁-C₃ alkyl), S02(C₁-C₃ alkyl), C₁-C₃ alkyl, O(C₃-C₄ cycloalkyl), S(C₃-C₄ cycloalkyl), SO₂(C₃-C₄ cycloalkyl), C₃-C₄ cycloalkyl, Cl, F, CN, or [C(═O)]₀₋₁NR^{x}R^{y};
R³ is NH(C₁-C₅ alkyl), N(C₁-C₅ alkyl)₂, NH(C₁-C₄ alkanediyl)₀₋₁(C₃-C₆ cycloalkyl), N(C₃-C₆ cycloalkyl)₂, NH(C₁-C₄ alkanediyl)₀₋₁(aryl), or a cyclic amine moiety having the structure 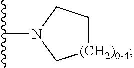
R⁵ is H, C₁-C₅ alkyl, C₂-C₅ alkenyl, C₃-C₆ cycloalkyl, halo, O(C₁-C₅ alkyl), (C₁-C₄ alkanediyl)OH, (C₁-C₄ alkanediyl)O(C₁-C₃ alkyl), phenyl, NH(C₁-C₅ alkyl), 5 or 6 membered heteroaryl, 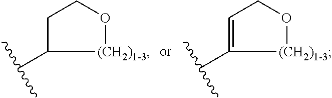
R^{x} and R^{y} are independently H or C₁-C₃ alkyl or R^{x} and R^{y} combine with the nitrogen to which they are bonded to form a 3- to 7-membered heterocycle;
m is 0 or 1;
and
n is 1, 2, or 3;
wherein in R¹, R², R³, and R⁵
an alkyl moiety, alkanediyl moiety, cycloalkyl moiety, or a moiety of the formula 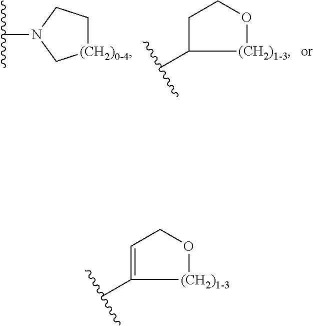
is optionally substituted with one or more substituents selected from OH, halo, CN, (C₁-C₃ alkyl), O(C₁-C₃ alkyl), C(═O)(C₁-C₃ alkyl), SO₂(C₁-C₃ alkyl), NR^{x}R^{y}, (C₁-C₄ alkanediyl)OH, (C₁-C₄ alkanediyl)O(C₁-C₃ alkyl); and
an alkyl, alkanediyl, cycloalkyl, or a moiety of the formula 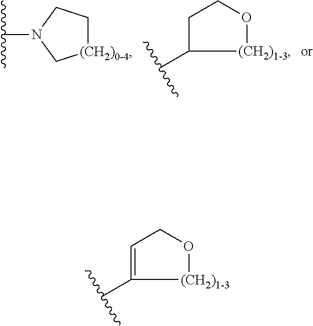
may have a CH₂ group replaced by O, SO₂, CF₂, C(═O), NH,
N[C(═O)]₀₋₁(C₁-C₃ alkyl),
N[C(═O)]₀₋₁(C₁-C₄ alkanediyl)CF₃,
N[C(═O)]₀₋₁(C₁-C₄ alkanediyl)OH,
or
N[C(═O)]₀₋₁(C₁-C₄ alkanediyl)₀₋₁(C₃-C₅ cycloalkyl).

2. A compound according to claim 1, having a structure according to formula (Ia):

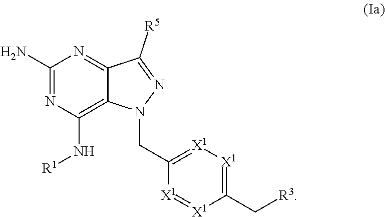

3. A compound according to claim 1, having a structure according to formula (Ib):

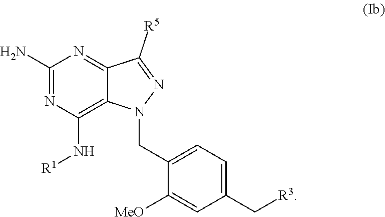

4. A compound according to claim 3, wherein R¹ is

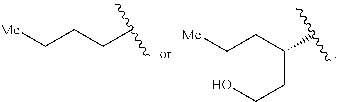 [SMILES: *CCCC ; *[C@@H](CCC)CCO]

5. A compound according to claim 3, wherein R³ is

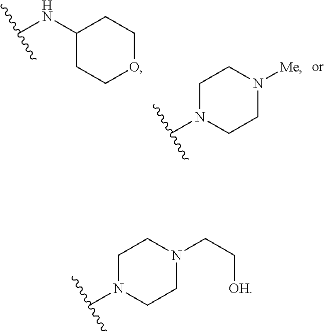 [SMILES: *NC1CCOCC1 ; *N1CCN(C)CC1 ; "or" ; *N1CCN(CCO)CC1]

6. A compound according to claim 1, wherein R⁵ is

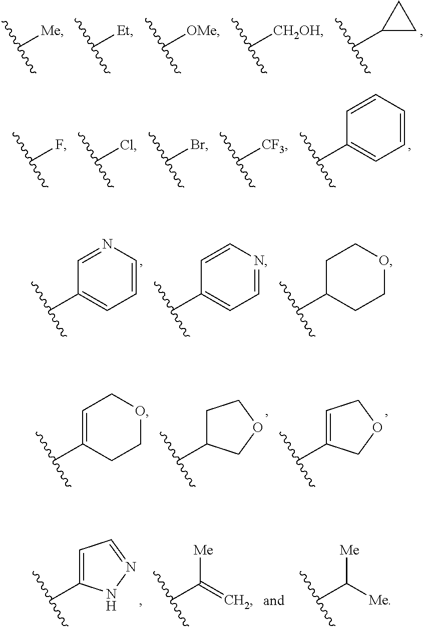

7. A compound according to claim 6, wherein
R¹ is 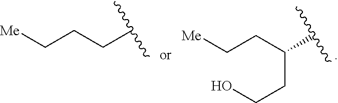 [SMILES: *CCCC ; *[C@@H](CCC)CCO]
R³ is 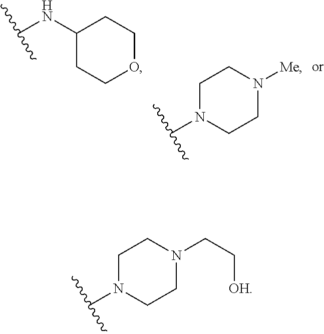 [SMILES: *NC1CCOCC1 ; *N1CCN(C)CC1 ; "or" ; *N1CCN(CCO)CC1]
and
R⁵ is

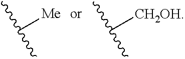

8. A compound having a structure according to formula (Ib) 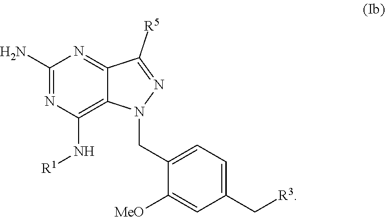
wherein
R¹ is 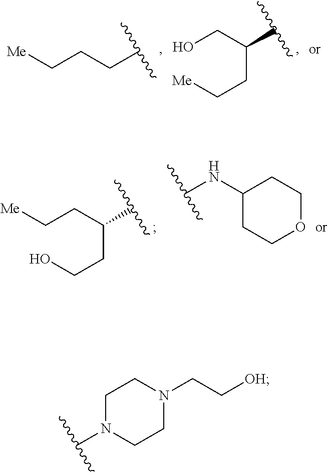 [SMILES: *CCCC ; *[C@H](CO)CCC ; *[C@@H](CCC)CCO ; *NC1CCOCC1 ; "or" ; *N1CCN(CCO)CC1]
R³ is
R⁵ is Me or CH₂OH.

9. A method of treating a cancer, comprising administering to a patient suffering from such cancer a therapeutically effective combination of an anti-cancer immunotherapy agent and a compound according to claim 1.

10. A method according to claim 9, wherein the anti-cancer immunotherapy agent is an antagonistic anti-CTLA-4, anti-PD-1, or anti-PD-L1 antibody.

11. A method according to claim 9, wherein the cancer is lung cancer (including non-small cell lung cancer), pancreatic cancer, kidney cancer, head and neck cancer, lymphoma (including Hodgkin's lymphoma), skin cancer (including melanoma and Merkel skin cancer), urothelial cancer (including bladder cancer), gastric cancer, hepatocellular cancer, or colorectal cancer.

12. A method according to claim 11, wherein the anti-cancer immunotherapy agent is ipilimumab, nivolumab, or pembrolizumab.

13. A compound according to claim 1, having a structure represented by formula (Ic)

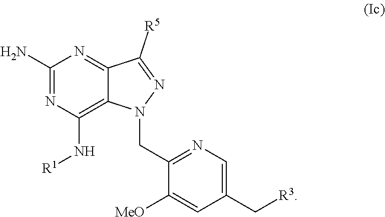

14. A method of treating a cancer, comprising administering to a patient suffering from such cancer a therapeutically effective combination of an anti-cancer immunotherapy agent and a compound according to claim 8.

15. A method according to claim 14, wherein the anti-cancer immunotherapy agent is an antagonistic anti-CTLA-4, anti-PD-1, or anti-PD-L1 antibody.

16. A method according to claim 14, wherein the cancer is lung cancer (including non-small cell lung cancer), pancreatic cancer, kidney cancer, head and neck cancer, lymphoma (including Hodgkin's lymphoma), skin cancer (including melanoma and Merkel skin cancer), urothelial cancer (including bladder cancer), gastric cancer, hepatocellular cancer, or colorectal cancer.

17. A method according to claim 16, wherein the anti-cancer immunotherapy agent is ipilimumab, nivolumab, or pembrolizumab.
